SEQUENCE LISTING (173 sequences)

SEQ ID NO: 1 - DNA, 276 bp, Artificial Sequence
  IIIsp::bpti::matureIII (initial fragment)
gtgaaaaaattattattcgcaattcctttagttgttcctttctattctggcgcccgtccggatttctgtctcgagccaccatacactgggccctgcaaagcgcgcatcatccgctatttctacaatgctaaagcaggcctgtgccagacctttgtatacggtggttgccgtgctaagcgtaacaactttaaatcggccgaagattgcatgcgtacctgcggtggcgccgctgaaactgttgaaagttgtttagcaaaaccccatacagaaaattca

SEQ ID NO: 2 - protein, 92 aa, Artificial Sequence
  IIIsp::bpti::matureIII (initial fragment)
MKKLLFAIPLVVPFYSGARPDFCLEPPYTGPCKARIIRYFYNAKAGLCQTFVYGGCRAKRNNFKSAEDCMRTCGGAAETVESCLAKPHTENS

SEQ ID NO: 3 - DNA, 285 bp, Artificial Sequence
  IIIsp::itiD1::mature III fusion gene
gtgaaaaaattattattcgcaattcctttagttgttcctttctattctggcgccaaagaagactcttgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtgggcgccgctgaaactgttgaaagttgtttagcaaaaccccatacagaaaattcattt

SEQ ID NO: 4 - protein, 95 aa, Artificial Sequence
  IIIsp::itiD1::mature III fusion gene
MKKLLFAIPLVVPFYSGAKEDSCQLGYSAGPCMGMTSRYFYNGTSMACETFQYGGCMGNGNNFVTEKECLQTCRTVGAAETVESCLAKPHTENSF

SEQ ID NO: 5 - protein, 58 aa, Artificial Sequence
  Consensus Kunitz domain
RPDFCLLPAETGPCRAMIPRFYYNAKSGKCEPFIYGGCGGNANNFKTEEECRRTCGGA

SEQ ID NO: 6 - protein, 58 aa, Bos Taurus [sequence omitted]

SEQ ID NO: 7 - protein, 58 aa, Artificial Sequence
  Epi-HNE-1
RPDFCLEPPYTGPCIAFFPRYFYNAKAGLCQTFVYGGCMGNGNNFKSAEDCMRTCGGA

SEQ ID NO: 8 - protein, 62 aa, Artificial Sequence
  Epi-HNE-2
EAEARPDFCLEPPYTGPCIAFFPRYFYNAKAGLCQTFVYGGCMGNGNNFKSAEDCMRTCGGA

SEQ ID NO: 9 - protein, 58 aa, Artificial Sequence
  EpiNE7
RPDFCLEPPYTGPCVAMFPRYFYNAKAGLCQTFVYGGCMGNGNNFKSAEDCMRTCGGA

SEQ ID NO: 10 - protein, 58 aa, Artificial Sequence
  EpiNE3
RPDFCLEPPYTGPCVGFFSRYFYNAKAGLCQTFVYGGCMGNGNNFKSAEDCMRTCGGA

SEQ ID NO: 11 - protein, 58 aa, Artificial Sequence
  EpiNE6
RPDFCLEPPYTGPCVGFFQRYFYNAKAGLCQTFVYGGCMGNGNNFKSAEDCMRTCGGA

SEQ ID NO: 12 - protein, 58 aa, Artificial Sequence
  EpiNE4
RPDFCLEPPYTGPCVAIFPRYFYNAKAGLCQTFVYGGCMGNGNNFKSAEDCMRTCGGA

SEQ ID NO: 13 - protein, 58 aa, Artificial Sequence
  EpiNE8
RPDFCLEPPYTGPCVAFFKRSFYNAKAGLCQTFVYGGCMGNGNNFKSAEDCMRTCGGA

SEQ ID NO: 14 - protein, 58 aa, Artificial Sequence
  EpiNE5
RPDFCLEPPYTGPCIAFFQRYFYNAKAGLCQTFVYGGCMGNGNNFKSAEDCMRTCGGA

SEQ ID NO: 15 - protein, 58 aa, Artificial Sequence
  EpiNE2
RPDFCLEPPYTGPCIALFKRYFYNAKAGLCQTFVYGGCMGNGNNFKSAEDCMRTCGGA

SEQ ID NO: 16 - protein, 58 aa, Homo sapiens
KEDSCQLGYSAGPCMGMTSRYFYNGTSMACETFQYGGCMGNGNNFVTEKDCLQTCRTV

SEQ ID NO: 17 - protein, 58 aa, Artificial Sequence
  BITI-E7-141
RPDFCQLGYSAGPCVAMFPRYFYNGTSMACQTFVYGGCMGNGNNFVTEKDCLQTCRGA

SEQ ID NO: 18 - protein, 58 aa, Artificial Sequence
  MUTT26A
RPDFCQLGYSAGPCVAMFPRYFYNGASMACQTFVYGGCMGNGNNFVTEKDCLQTCRGA

SEQ ID NO: 19 - protein, 58 aa, Artificial Sequence
  MUTQE
RPDFCQLGYSAGPCVAMFPRYFYNGTSMACETFVYGGCMGNGNNFVTEKDCLQTCRGA

SEQ ID NO: 20 - protein, 58 aa, Artificial Sequence
  MUT1619
RPDFCQLGYSAGPCVGMFSRYFYNGTSMACQTFVYGGCMGNGNNFVTEKDCLQTCRGA

SEQ ID NO: 21 - protein, 58 aa, Artificial Sequence
  ITI-D1E7
KEDSCQLGYSAGPCVAMFPRYFYNGTSMACETFQYGGCMGNGNNFVTEKDCLQTCRGA

SEQ ID NO: 22 - protein, 58 aa, Artificial Sequence
  AMINO1
KEDFCQLGYSAGPCVAMFPRYFYNGTSMACETFQYGGCMGNGNNFVTEKDCLQTCRGA

SEQ ID NO: 23 - protein, 58 aa, Artificial Sequence
  AMINO2
KPDSCQLGYSAGPCVAMFPRYFYNGTSMACETFQYGGCMGNGNNFVTEKDCLQTCRGA

SEQ ID NO: 24 - protein, 58 aa, Artificial Sequence
  MUTP1
RPDFCQLGYSAGPCIGMFSRYFYNGTSMACETFQYGGCMGNGNNFVTEKDCLQTCRGA

SEQ ID NO: 25 - protein, 58 aa, Homo sapiens
TVAACNLPIVRGPCRAFIQLWAFDAVKGKCVLFPYGGCQGNGNKFYSEKECREYCGVP

SEQ ID NO: 26 - protein, 56 aa, Artificial Sequence
  Epi-HNE-3
AACNLPIVRGPCIAFFPRWAFDAVKGKCVLFPYGGCQGNGNKFYSEKECREYCGVP

SEQ ID NO: 27 - protein, 56 aa, Artificial Sequence
  Epi-HNE-4
EACNLPIVRGPCIAFFPRWAFDAVKGKCVLFPYGGCQGNGNKFYSEKECREYCGVP

SEQ ID NO: 28 - protein, 58 aa, Homo sapiens
VREVCSEQAETGPCRAMISRWYFDVTEGKCAPFFYGGCGGNRNNFDTEEYCMAVCGSA

SEQ ID NO: 29 - protein, 58 aa, Artificial Sequence
  DPI.1.1
VREVCSEQAYTGPCIAFFPRYYFDVTEGKCQTFVYGGCMGNGNNFDTEEYCMAVCGSA

SEQ ID NO: 30 - protein, 58 aa, Artificial Sequence
  DPI.1.2
VREVCSEQAETGPCIAMFSRWYFDVTEGKCAPFVYGGCGGNRNNFDTEEYCMAVCGSA

SEQ ID NO: 31 - protein, 58 aa, Artificial Sequence
  DPI.1.3
VREVCSEQAETGPCIAFFSRWYFDVTEGKCATFVYGGCMGNRNNFDTEEYCMAVCGSA

SEQ ID NO: 32 - protein, 58 aa, Homo sapiens
NAEICLLPLDYGPCRALLLRYYYDRYTQSCRQFLYGGCEGNANNFYTWEACDDACWRI

SEQ ID NO: 33 - protein, 58 aa, Artificial Sequence
  DPI.2.1
NAEICLLPLYTGPCIAFFPRYYYDRYTQSCQTFVYGGCMGNANNFYTWEACDDACWRI

SEQ ID NO: 34 - protein, 58 aa, Artificial Sequence
  DPI.2.2
NAEICLLPLDYGPCIALFLRYYYDRYTQSCRQFVYGGCEGNANNFYTWEACDDACWRI

SEQ ID NO: 35 - protein, 58 aa, Artificial Sequence
  DPI.2.3
NAEICLLPLDTGPCIAFFLRYYYDRYTQSCQTFVYGGCMGNANNFYTWEACDDACWRI

SEQ ID NO: 36 - protein, 61 aa, Homo sapiens
VPKVCRLQVSVDDQCEGSTEKYFFNLSSMTCEKFFSGGCHRNRIENRFPDEATCMGFCAPK

SEQ ID NO: 37 - protein, 58 aa, Artificial Sequence
  DPI.3.1
VPKVCRLQVVRGPCIAFFPRWFFNLSSMTCVLFPYGGCQGNGNRFPDEATCMGFCAPK

SEQ ID NO: 38 - protein, 61 aa, Artificial Sequence
  DPI.3.2
VPKVCRLQVSVDDQCIGSFEKYFFNLASMTCETFVSGGCHRNRIENRFPDEATCMGFCAPK

SEQ ID NO: 39 - protein, 58 aa, Artificial Sequence
  DPI.3.3
VPKVCRLQVVAGPCIGFFKRYFFALSSMTCETFVSGGCHRNRNRFPDEATCMGFCAPK

SEQ ID NO: 40 - protein, 58 aa, Homo sapiens
IPSFCYSPKDEGLCSANVTRYYFNPRYRTCDAFTYTGCGGNDNNFVSREDCKRACAKA

SEQ ID NO: 41 - protein, 58 aa, Artificial Sequence
  DPI.4.1
IPSFCYSPKSAGPCVAMFPRYYFNPRYRTCETFVYGGCMGNGNNFVSREDCKRACAKA

SEQ ID NO: 42 - protein, 58 aa, Artificial Sequence
  DPI.4.2
IPSFCYSPKDEGLCIAFFTRYYFNPRYRTCDAFTYTGCGGNDNNFVSREDCKRACAKA

SEQ ID NO: 43 - protein, 58 aa, Artificial Sequence
  DPI.4.3
IPSFCYSPKDTGPCIAFFTRYYFNPRYRTCDTFVYGGCGGNDNNFVSREDCKRACAKA

SEQ ID NO: 44 - protein, 58 aa, Homo sapiens
MHSFCAFKADDGPCKAIMKRFFFNIFTRQCEEFIYGGCEGNQNRFESLEECKKMCTRD

SEQ ID NO: 45 - protein, 58 aa, Artificial Sequence
  DPI.5.1
MHSFCAFKASAGPCVAMFPRYFFNIFTRQCETFVYGGCMGNGNRFESLEECKKMCTRD

SEQ ID NO: 46 - protein, 58 aa, Artificial Sequence
  DPI.5.2
MHSFCAFKADDGPCIAIFKRFFFNIFTRQCEEFIYGGCEGNQNRFESLEECKKMCTRD

SEQ ID NO: 47 - protein, 58 aa, Artificial Sequence
  DPI.5.3
MHSFCAFKAYTGPCIAFFKRFFFNIFTRQCETFIYGGCEGNQNRFESLEECKKMCTRD

SEQ ID NO: 48 - protein, 58 aa, Homo sapiens
KPDFCFLEEDPGICRGYITRYFYNNQTKQCERFKYGGCLGNMNNFETLEECKNICEDG

SEQ ID NO: 49 - protein, 58 aa, Artificial Sequence
  DPI.6.1
KPDFCFLEESAGPCVAMFPRYFYNNQTKQCETFVYGGCMGNGNNFETLEECKNICEDG

SEQ ID NO: 50 - protein, 58 aa, Artificial Sequence
  DPI.6.2
KPDFCFLEEDPGICVGYFTRYFYNNQTKQCERFKYGGCLGNMNNFETLEECKNICEDG

SEQ ID NO: 51 - protein, 58 aa, Artificial Sequence
  DPI.6.3
KPDFCFLEEDPGICVGFFTRYFYNNQTKQCERFVYGGCLGNMNNFETLEECKNICEDG

SEQ ID NO: 52 - protein, 58 aa, Artificial Sequence
  DPI.6.4
KPDFCFLEEDPGICVGFFTRYFYNAQTKQCERFVYGGCLGNMNNFETLEECKNICEDG

SEQ ID NO: 53 - protein, 58 aa, Artificial Sequence
  DPI.6.5
KPDFCFLEEDPGPCVGFFQRYFYNAQTKQCERFVYGGCQGNMNNFETLEECKNICEDG

SEQ ID NO: 54 - protein, 58 aa, Artificial Sequence
  DPI.6.6
KPDFCFLEEDPGPCVGFFTRYFYNNQTKQCERFVYGGCQGNMNNFETLEECKNICEDG

SEQ ID NO: 55 - protein, 58 aa, Artificial Sequence
  DPI.6.7
KPDFCFLEEDPGPCIGFFPRYFYNNQTKQCERFVYGGCQGNMNNFETLEECKNICEDG

SEQ ID NO: 56 - protein, 58 aa, Homo sapiens
GPSWCLTPADRGLCRANENRFYYNSVIGKCRPFKYSGCGGNENNFTSKQECLRACKKG

SEQ ID NO: 57 - protein, 58 aa, Artificial Sequence
  DPI.7.1
GPSWCLTPAVRGPCIAFFPRWYYNSVIGKCVLFPYGGCQGNGNNFTSKQECLRACKKG

SEQ ID NO: 58 - protein, 58 aa, Artificial Sequence
  DPI.7.2
GPSWCLTPADRGLCVANFNRFYYNSVIGKCRPFKYSGCGGNENNFTSKQECLRACKKG

SEQ ID NO: 59 - protein, 58 aa, Artificial Sequence
  DPI.7.3
GPSWCLTPADRGLCVAFFNRFYYNSVIGKCRPFKYSGCGGNENNFKSKQECLRACKKG

SEQ ID NO: 60 - protein, 58 aa, Artificial Sequence
  DPI.7.4
GPSWCLTPAVRGPCVAFFNRFYYNSVIGKCRPFKYGGCGGNENNFKSKQECLRACKKG

SEQ ID NO: 61 - protein, 58 aa, Artificial Sequence
  DPI.7.5
GPSWCLTPADRGPCIAFFPRWYYNSVIGKCQTFVYGGCGGNENNFASKQECLRACKKG

SEQ ID NO: 62 - protein, 58 aa, Homo sapiens
ETDICKLPKDEGTCRDFILKWYYDPNTKSCARFWYGGCGGNENKFGSQKECEKVCAPV

SEQ ID NO: 63 - protein, 58 aa, Artificial Sequence
  DPI.8.1
ETDICKLPKVRGPCIAFFPRWYYDPNTKSCVLFPYGGCQGNGNKFGSQKECEKVCAPV

SEQ ID NO: 64 - protein, 58 aa, Artificial Sequence
  DPI.8.2
ETDICKLPKDEGTCIAFFLKWYYDPNTKSCARFVYGGCGGNENKFGSQKECEKVCAPV

SEQ ID NO: 65 - protein, 58 aa, Artificial Sequence
  DPI.8.3
ETDICKLPKDEGPCIAFFLRWYYDPNTKSCARFVYGGCGGNENKFGSQKECEKVCAPV

SEQ ID NO: 66 - protein, 58 aa, Homo sapiens
LPNVCAFPMEKGPCQTYMTRWFFNFETGECELFAYGGCGGNSNNFLRKEKCEKFCKFT

SEQ ID NO: 67 - protein, 58 aa, Artificial Sequence
  DPI.9.1
LPNVCAFPMVRGPCIAFFPRWFFNFETGECVLFVYGGCQGNGNNFLRKEKCEKFCKFT

SEQ ID NO: 68 - protein, 58 aa, Artificial Sequence
  DPI.9.2
LPNVCAFPMEKGPCIAYFTRWFFNFETGECELFAYGGCGGNSNNFLRKEKCEKFCKFT

SEQ ID NO: 69 - protein, 58 aa, Artificial Sequence
  DPI.9.3
LPNVCAFPMEKGPCIAYFPRWFFNFETGECVLFVYGGCGGNSNNFLRKEKCEKFCKFT

SEQ ID NO: 70 - DNA, 8157 bp, Artificial Sequence
  Plasmid pHIL-D2
agatcgcggccgcgatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggtcatgtttgtttatttccgaatgcaacaagctccgcattacacccgaacatcactccagatgagggctttctgagtgtggggtcaaatagtttcatgttcccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaacccggtggcacctgtgccgaaacgcaaatggggaaacaacccgctttttggatgattatgcattgtcctccacattgtatgcttccaagattctggtgggaatactgctgatagcctaacgttcatgatcaaaatttaactgttctaacccctacttgacaggcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgaggaattcgccttagacatgactgttcctcagttcaagttgggcattacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattatacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatcgcggccgcgatctcgaataataactgttatttttcagtgttcccgatctgcgtctatttcacaataccaacatgagtcagcttatcgatgataagctgtcaaacatgagaattaattcgatgataagctgtcaaacatgagaaatcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 71 - DNA, 8584 bp, Artificial Sequence
  Plasmid pHIL-D2 (MFalphaPrePro::EPI-HNE-3)
agatcgcggccgcgatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggtcatgtttgtttatttccgaatgcaacaagctccgcattacacccgaacatcactccagatgagggctttctgagtgtggggtcaaatagtttcatgttcccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaacccggtggcacctgtgccgaaacgcaaatggggaaacaacccgctttttggatgattatgcattgtcctccacattgtatgcttccaagattctggtgggaatactgctgatagcctaacgttcatgatcaaaatttaactgttctaacccctacttgacaggcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagattcccatctatcttcactgctgttttgttcgctgcttcctctgctttggctgctccagttaacaccactactgaagacgagactgctcaaattcctgctgaggctgtcatcggttactctgacttggaaggtgacttcgacgtcgctgttttgccattctctaactctactaacaacggtttgttgttcatcaacactaccatcgcttctatcgctgctaaggaggaaggtgtttccttggacaagagagctgcttgtaacttgccaatcgtcagaggtccatgcattgctttcttcccaagatgggctttcgacgctgttaagggtaagtgcgtcttgttcccatacggtggttgtcaaggtaacggtaacaagttctactctgagaaggagtgtagagagtactgtggtgttccatagtaagaattcgccttagacatgactgttcctcagttcaagttgggcattacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattatacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatcgcggccgcgatctcgaataataactgttatttttcagtgttcccgatctgcgtctatttcacaataccaacatgagtcagcttatcgatgataagctgtcaaacatgagaattaattcgatgataagctgtcaaacatgagaaatcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 72 - protein, 141 aa, Artificial Sequence
  Plasmid pHIL-D2 (MFalphaPrePro::EPI-HNE-3)
MRFPSIFTAVLFAASSALAAPVNTTTEDETAQIPAEAVIGYSDLEGDFDVAVLPFSNSTNNGLLFINTTIASIAAKEEGVSLDKRAACNLPIVRGPCIAFFPRWAFDAVKGKCVLFPYGGCQGNGNKFYSEKECREYCGVP

SEQ ID NO: 73 - DNA, 444 bp, Artificial Sequence
  BstBI-AatII-EcoRI cassette for expression of Epi-HNE-4
ttcgaaacgatgagattcccatctatcttcactgctgttttgttcgctgcttcctctgctttggctgctccagttaacaccactactgaagacgagactgctcaaattcctgctgaggctgtcatcggttactctgacttggaaggtgacttcgacgtcgctgttttgccattctctaactctactaacaacggtttgttgttcatcaacactaccatcgcttctatcgctgctaaggaggaaggtgtttccttggacaagagagaggcttgtaacttgccaatcgtcagaggtccatgcattgctttcttcccaagatgggctttcgacgctgttaagggtaagtgcgtcttgttcccatacggtggttgtcaaggtaacggtaacaagttctactctgagaaggagtgtagagagtactgtggtgttccatagtaagaattc

SEQ ID NO: 74 - protein, 141 aa, Artificial Sequence
  BstBI-AatII-EcoRI cassette for expression of Epi-HNE-4
MRFPSIFTAVLFAASSALAAPVNTTTEDETAQIPAEAVIGYSDLEGDFDVAVLPFSNSTNNGLLFINTTIASIAAKEEGVSLDKREACNLPIVRGPCIAFFPRWAFDAVKGKCVLFPYGGCQGNGNKFYSEKECREYCGVP

SEQ ID NO: 75 - DNA, 8590 bp, Artificial Sequence
  pD2pick (MFalphaPrePro::EPI-NHE-3) circular dsDNA
agatcgcggccgcgatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggtcatgtttgtttatttccgaatgcaacaagctccgcattacacccgaacatcactccagatgagggctttctgagtgtggggtcaaatagtttcatgttcccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaacccggtggcacctgtgccgaaacgcaaatggggaaacaacccgctttttggatgattatgcattgtcctccacattgtatgcttccaagattctggtgggaatactgctgatagcctaacgttcatgatcaaaatttaactgttctaacccctacttgacaggcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagattcccatctatcttcactgctgttttgttcgctgcttcctctgctttggctgctccagttaacaccactactgaagacgagactgctcaaattcctgctgaggctgtcatcggttactctgacttggaaggtgacttcgacgtcgctgttttgccattctctaactctactaacaacggtttgttgttcatcaacactaccatcgcttctatcgctgctaaggaggaaggtgtttccttggacaagagagctgcttgtaacttgccaatcgtcagaggtccatgcattgctttcttcccaagatgggctttcgacgctgttaagggtaagtgcgtcttgttcccatacggtggttgtcaaggtaacggtaacaagttctactctgagaaggagtgtagagagtactgtggtgttccatagtaagaattcgccttagacatgactgttcctcagttcaagttgggcattacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattatacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatcgcggccgcgatctcgaataataactgttatttttcagtgttcccgatctgcgtctatttcacaataccaacatgagtcagcttatcgatgataagctgtcaaacatgagaattaattcgatgataagctgtcaaacatgagaaatcttgaagacgaaagggcctcgtgatacgcctatttttataggttaatgtcatgataataatggtttcttagacgtacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 76 - protein, 141 aa, Artificial Sequence; identical to SEQ ID NO: 72
  EPI-HNE-3 fusion protein

SEQ ID NO: 77 - protein, 147 aa, Homo sapiens
AVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 78 - DNA, 249 bp, Artificial Sequence
  M13_III_signal::Human_LACI-D2::mature_M13_III
atgaagaagcttctcttcgccattcctctggtggtacctttctattccggcgccaagcctgacttctgcttcctcgaggaggatcccgggatttgccgcggttatattacgcgttatttctataataaccagactaagcaatgtgagcggttcaagtatggtggttgcctaggtaatatgaacaacttcgagactctagaagagtgtaagaacatatgtgaggatggtggtgctgagactgttgagtct

SEQ ID NO: 79 - protein, 83 aa, Artificial Sequence
  LACI-D2 fusion protein
MKKLLFAIPLVVPFYSGAKPDFCFLEEDPGICRGYITRYFYNNQTKQCERFKYGGCLGNMNNFETLEECKNICEDGGAETVES

SEQ ID NO: 80 - DNA, 189 bp, Artificial Sequence
  laci-d1 with cloning sites
gcggccgagatgcattccttctgcgctttcaaagctgatgacggtccgtgtaaagctatcatgaaacgtttcttcttcaacattttcacgcgtcagtgcgaggaattcatttacggtggttgtgaaggtaaccagaaccggttcgaatctctagaggaatgtaagaagatgtgcactcgtgacggcgcc

SEQ ID NO: 81 - protein, 63 aa, Artificial Sequence
  laci-d1 with linkers
AAEMHSFCAFKADDGPCKAIMKRFFFNIFTRQCEEFIYGGCEGNQNRFESLEECKKMCTRDGA

SEQ ID NO: 82 - DNA, 189 bp, Artificial sequence
  LACI-D1 hNE library
  misc_feature (37)..(37): n is a, c, g, or t
  misc_feature (38)..(38): n is a or g
  misc_feature (40)..(40): n is a or g
  misc_feature (41)..(41): n is a, c or g
  misc_feature (42)..(42): n is c or g
  misc_feature (47)..(47): n is a, c, g, or t
  misc_feature (52)..(52): n is a or g
  misc_feature (56)..(56): n is c or g
  misc_feature (58)..(58): n is a, c, g, or t
  misc_feature (64)..(64): n is a or c
  misc_feature (65)..(65): n is a, c, g, or t
  misc_feature (66)..(66): n is c or g
  misc_feature (71)..(71): n is a, g or t
  misc_feature (72)..(72): n is c or g
  misc_feature (100)..(100): n is c or g
  misc_feature (101)..(101): n is a or t
  misc_feature (103)..(103): n is a, c or g
  misc_feature (104)..(104): n is a, c or t
  misc_feature (109)..(109): n is a, c or g
  misc_feature (110)..(110): n is a, c or t
  misc_feature (124)..(124): n is a, c or g
  misc_feature (125)..(125): n is a, c or t
  misc_feature (128)..(128): n is c or g
  misc_feature (133)..(133): n is c or g
  misc_feature (134)..(134): n is a or g
gcggccgagatgcattccttctgcgctttcaaagctnntnnnggtcnttgtnttgntntcttcnnncgttnnttcttcaacattttcacgcgtcagtgcnngnnattcnnatacggtggttgtnnggntaacnngaaccggttcgaatctctagaggaatgtaagaagatgtgcactcgtgacggcgcc

SEQ ID NO: 83 - protein, 63 aa, Artificial Sequence
  LACI-D1 hNE library
  misc_feature (13)..(13): Xaa is Cys, Arg, Ser, Gly, Tyr, His, Asp or Asn
  misc_feature (14)..(14): Xaa is Thr, Asn, Lys, Arg, Ser, Ala, Glu, Gly or Asp
  misc_feature (16)..(16): Xaa is His, Arg, Pro or Leu
  misc_feature (18)..(18): Xaa is Val or Ile
  misc_feature (19)..(19): Xaa is Ala or Gly
  misc_feature (20)..(20): Xaa is Phe, Leu, Ile or Val
  misc_feature (22)..(22): Xaa is Ser, Thr, Asn, Ile, Met, Gln, His, Leu, Pro, Lys or Arg
  misc_feature (24)..(24): Xaa is Cys, Tyr, Trp, Phe or Leu
  misc_feature (34)..(34): Xaa is Leu, Gln, Glu or Val
  misc_feature (35)..(35): Xaa is Gln, Leu, Pro, Thr, Lys, Val, Ile, Glu or Ala
  misc_feature (37)..(37): Xaa is Gln, Leu, Pro, Thr, Lys, Val, Glu, Ile or Ala
  misc_feature (42)..(42): Xaa is Gln, Leu, Pro, Thr, Lys, Val, Met, Glu or Ala
  misc_feature (43)..(43): Xaa is Gly or Ala
  misc_feature (45)..(45): Xaa is Glu, Gly, Gln or Arg
AAEMHSFCAFKAXXGXCXXXFXRXFFNIFTRQCXXFXYGGCXXNXNRFESLEECKKMCTRDGA

SEQ ID NO: 84 - DNA, 201 bp, Artificial sequence
  LACI-D2 hNE library
  misc_feature (34)..(34): n is a, c, g, or t
  misc_feature (35)..(35): n is a or g
  misc_feature (37)..(38): n is a, c or g
  misc_feature (39)..(39): n is c or g
  misc_feature (43)..(43): n is a or c
  misc_feature (44)..(44): n is a, c, g, or t
  misc_feature (49)..(49): n is a or g
  misc_feature (53)..(53): n is c or g
  misc_feature (55)..(55): n is a, c, g, or t
  misc_feature (56)..(56): n is a or t
  misc_feature (61)..(61): n is a or c
  misc_feature (62)..(62): n is a, c, g, or t
  misc_feature (63)..(63): n is c or g
  misc_feature (68)..(68): n is a, g or t
  misc_feature (69)..(69): n is c or g
  misc_feature (97)..(97): n is c or g
  misc_feature (98)..(98): n is a or t
  misc_feature (100)..(100): n is a, c or g
  misc_feature (101)..(101): n is a, c, g, or t
  misc_feature (106)..(106): n is a, c, or g
  misc_feature (107)..(107): n is a, c, or t
  misc_feature (121)..(121): n is a, c, or g
  misc_feature (122)..(122): n is a, c, or t
  misc_feature (125)..(125): n is c or g
  misc_feature (130)..(130): n is a, c or g
  misc_feature (131)..(131): n is c, g or t
ggcgccaagcctgacttctgcttcctcgaggagnntnnngggnnttgcnttgntnnttttnnncgttnnttctataataaccaggctaagcaatgtnngnnattcnnatatggtggttgcnnggntaatnngaacaacttcgagactctagaagagtgtaagaacatatgtgaggatggtggtgctgagactgttgagtct

SEQ ID NO: 85 - protein, 67 aa, Artificial Sequence
  LACI-D2 hNE library
  misc_feature (12)..(12): Xaa is Cys, Arg, Ser, Gly, Tyr, His, Asp or Asn
  misc_feature (13)..(13): Xaa is Pro, His, Thr, Asn, Lys, Arg, Ser, Ala, Glu, Gly, Asp or Gln
  misc_feature (15)..(15): Xaa is His, Arg, Pro, Leu, Asn, Ser, Ile or Thr
  misc_feature (17)..(17): Xaa is Val or Ile
  misc_feature (18)..(18): Xaa is Gly or Ala
  misc_feature (19)..(19): Xaa is Phe, Leu, Ile, Val, Tyr, His, Asn or Asp
  misc_feature (21)..(21): Xaa is Ile, Asn, Gln, Met, Leu, His, Lys, Pro, Thr or Arg
  misc_feature (23)..(23): Xaa is Cys, Phe, Leu, Tyr or Trp
  misc_feature (33)..(33): Xaa is Leu, Gln, Glu or Val
  misc_feature (34)..(34): Xaa is Gln, Gly, Leu, Pro, Thr, Lys, Val, Ile, Glu, Ala or Arg
  misc_feature (36)..(36): Xaa is Gln, Leu, Pro, Thr, Val, Glu, Ile, Ala or Lys
  misc_feature (41)..(41): Xaa is Gln, Pro, Thr, Lys, Val, Met, Glu, Ala or Leu
  misc_feature (42)..(42): Xaa is Gly or Ala
  misc_feature (44)..(44): Xaa is Arg, Gly, Lys, Glu, Leu, Gln, Met or Val
GAKPDFCFLEEXXGXCXXXFXRXFYNNQAKQCXXFXYGGCXXNXNNFETLEECKNICEDGGAETVES

SEQ ID NO: 86 - protein, 51 aa, Artificial Sequence
  definition of aprotonin-like Kunitz domain (p. 11)
  misc_feature (2)..(7): Xaa is any amino acid
  misc_feature (9)..(9): Xaa is any amino acid
  misc_feature (11)..(18): Xaa is any amino acid
  misc_feature (19)..(19): Xaa is any Tyr or Phe
  misc_feature (20)..(25): Xaa is any amino acid
  misc_feature (27)..(28): Xaa is any amino acid
  misc_feature (30)..(30): Xaa is any amino acid
  misc_feature (31)..(31): Xaa is Tyr, Trp or Phe
  misc_feature (32)..(32): Xaa is any amino acid
  misc_feature (35)..(38): Xaa is any amino acid
  misc_feature (39)..(39): Xaa is Asn or Gly
  misc_feature (40)..(40): Xaa is any amino acid
  misc_feature (41)..(41): Xaa is Phe or Tyr
  misc_feature (42)..(46): Xaa is any amino acid
  misc_feature (48)..(50): Xaa is any amino acid
CXXXXXXGXCXXXXXXXXXXXXXXXCXXFXXXGCXXXXXXXXXXXXCXXXC

SEQ ID NO: 87 - protein, 58 aa, Bos Taurus [sequence omitted]

SEQ ID NO: 88 - protein, 58 aa, Artificial Sequence
  Engineered B-PTI from MARK87
RPDFCLEPPYTGPTKARIIRYFYNAKAGLCQTFVYGGTRAKRNNFKSAEDCMRTCGGA

SEQ ID NO: 89 - protein, 58 aa, Artificial Sequence
  Engineered B-PTI from MARK87
RPDFCLEPPYTGPAKARIIRYFYNAKAGLCQTFVYGGARAKRNNFKSAEDCMRTCGGA

SEQ ID NO: 90 - protein, 67 aa, Bos taurus (Bovine Colostrum)
FQTPPDLCQLPQARGPCKAALLRYFYNSTSNACEPFTYGGCQGNNNNFETTEMCLRICEPPQQTDKS

SEQ ID NO: 91 - protein, 60 aa, Bos Taurus (Bovine serum)
TERPDFCLEPPYTGPCKAAMIRYFYNAKAGFCETFVYGGCRAKSNNFKSAEDCMRTCGGA

SEQ ID NO: 92 - protein, 58 aa, Artificial Sequence
  Semisynthetic BPTI, TSCH87
RPDFCLEPPYTGPCVARIIRYFYNAKAGLCQTFVYGGCRAKRNNFKSAEDCMRTCGGA

SEQ ID NO: 93 - protein, 58 aa, Artificial Sequence
  Semisynthetic BPTI, TSCH87
RPDFCLEPPYTGPCGARIIRYFYNAKAGLCQTFVYGGCRAKRNNFKSAEDCMRTCGGA

SEQ ID NO: 94 - protein, 58 aa, Artificial Sequence
  Semisynthetic BPTI, TSCH87
RPDFCLEPPYTGPCAARIIRYFYNAKAGLCQTFVYGGCRAKRNNFKSAEDCMRTCGGA

SEQ ID NO: 95 - protein, 58 aa, Artificial Sequence
  Semisynthetic BPTI, TSCH87
RPDFCLEPPYTGPCLARIIRYFYNAKAGLCQTFVYGGCRAKRNNFKSAEDCMRTCGGA

SEQ ID NO: 96 - protein, 58 aa, Artificial Sequence
  Semisynthetic BPTI, TSCH87
RPDFCLEPPYTGPCIARIIRYFYNAKAGLCQTFVYGGCRAKRNNFKSAEDCMRTCGGA

SEQ ID NO: 97 - protein, 58 aa, Artificial Sequence
  Engineered BPTI, AUER87
RPDFCLEPPYTGPCKARIIRYFYNAKAGLCQTFVYGGCRAKRNNFKSAEDCERTCGGA

SEQ ID NO: 98 - protein, 60 aa, Dendroaspis polylepis polylepis (Black mamba venom I)
QPLRKLCILHRNPGRCYQKIPAFYYNQKKKQCEGFTWSGCGGNSNRFKTIEECRRTCIRK

SEQ ID NO: 99 - protein, 57 aa, Dendroaspis polylepis polylepis (Black mamba venom K)
AAKYCKLPLRIGPCKRKIPSFYYKWKAKQCLPFDYSGCGGNANRFKTIEECRRTCVG

SEQ ID NO: 100 - protein, 57 aa, Hemachatus hemachates
RPDFCELPAETGLCKAYIRSFHYNLAAQQCLQFIYGGCGGNANRFKTIDECRRTCVG

SEQ ID NO: 101 - protein, 57 aa, Naja nivea
RPRFCELPAETGLCKARIRSFHYNRAAQQCLEFIYGGCGGNANRFKTIDECHRTCVG

SEQ ID NO: 102 - protein, 60 aa, Vipera russelli
HDRPTFCNLPPESGRCRGHIRRIYYNLESNKCKVFFYGGCGGNANNFETRDECRETCGGK

SEQ ID NO: 103 - protein, 64 aa, Caretta sp. (Red sea turtle egg white)
  misc_feature (1)..(1): Xaa is Glu or Gln
XGDKRDICRLPPEQGPCKGRLPRYFYNPASRMCESFIYGGCKGNKNNFKTKAECVRACRPPERP

SEQ ID NO: 104 - protein, 58 aa, Helix pomania
  misc_feature (1)..(1): Xaa is Glu or Gln
XGRPSFCNLPAETGPCKASIRQYYYNSKSGGCQQFIYGGCRGNQNRFDTTQQCQGVCV

SEQ ID NO: 105 - protein, 57 aa, Dendroaspis angusticeps (Eastern green mamba C13 S1 C3 toxin)
AAKYCKLPVRYGPCKKKFPSFYYNWKAKQCLPFNYSGCGGNANRFKTIEECRRTCVG

SEQ ID NO: 106 - protein, 59 aa, Dendroaspis angusticeps (Eastern green mamba C13 S2 C3 toxin)
  misc_feature (1)..(1): Xaa is Glu or Gln
XPRRKLCILHRNPGRCYDKIPAFYYNQKKKQCERFDWSGCGGNSNRFKTIEECRRTCIG

SEQ ID NO: 107 - protein, 57 aa, Dendroaspis polylepis polylepis (Black mamba B toxin)
RPYACELIVAAGPCMFFISAFYYSKGANKCYPFTYSGCRGNANRFKTIEECRRTCVV

SEQ ID NO: 108 - protein, 59 aa, Dendroaspis polylepis polylepis (Black mamba E toxin)
LQHRTFCKLPAEPGPCKASIPAFYYNWAAKKCQLFHYGGCKGNANRFSTIEKCRHACVG

SEQ ID NO: 109 - protein, 61 aa, Vipera ammodytes TI toxin
  misc_feature (1)..(1): Xaa is Glu or Gln
XDHPKFCYLPADPGRCKAHIPRFYYDSASNKCNKFIYGGCPGNANNFKTWDECRQTCGASA

SEQ ID NO: 110 - protein, 62 aa, Vipera ammodytes CTI toxin
RDRPKFCYLPADPGRCLAYMPRFYYNPASNKCEKFIYGGCRGNANNFKTWDECRHTCVASGI

SEQ ID NO: 111 - protein, 62 aa, Bungarus fasciatus VIII B toxin
KNRPTFCNLLPETGRCNALIPAFYYNSHLHKCQKFNYGGCGGNANNFKTIDECQRTCAAKYG

SEQ ID NO: 112 - protein, 59 aa, Anemonia sulcata
INGDCELPKVVGPCRARFPRYYYNSSSKRCEKFIYGGCGGNANNFHTLEECEKVCGVRS

SEQ ID NO: 113 - protein, 56 aa, Homo sapiens
KEDSCQLGYSAGPCMGMTSRYFYNGTSMACETFQYGGCMGNGNNFVTEKECLQTCR

SEQ ID NO: 114 - protein, 61 aa, Homo sapiens
TVAACNLPVIRGPCRAFIQLWAFDAVKGKCVLFPYGGCQGNGNKFYSEKECREYCGVPGDE

SEQ ID NO: 115 - protein, 60 aa, Bungarus multicinctus (beta bungarotoxin B1)
RQRHRDCDKPPDKGNCGPVRAFYYDTRLKTCKAFQYRGCDGDHGNFKTETLCRCECLVYP

SEQ ID NO: 116 - protein, 60 aa, Bungarus multicinctus (beta bungarotoxin B2)
RKRHPDCDKPPDTKICQTVRAFYYKPSAKRCVQFRYGGCDGDHGNFKSDHLCRCECELYR

SEQ ID NO: 117 - protein, 58 aa, Bos taurus
RPDFCLEPPYTGPCKAKMIRYFYNAKAGFCETFVYGGCKAKSNNFRSAEDCMRTCGGA

SEQ ID NO: 118 - protein, 61 aa, Tachypleus tridentatus
TERGFLDCTSPPVTGPCRAGFKRYNYNTRTKQCEPFKYGGCKGNGNRYKSEQDCLDACSGF

SEQ ID NO: 119 - protein, 63 aa, Bombyx mori
DEPTTDLPICEQAFGDAGLCFGYMKLYSYNQETKNCEEFIYGGCQGNDNRFSTLAECEQKCIN

SEQ ID NO: 120 - protein, 56 aa, Bos taurus
KADSCQLDYSQGPCLGLFKRYFYNGTSMACETFLYGGCMGNLNNFLSQKECLQTCR

SEQ ID NO: 121 - protein, 61 aa, Bos taurus
TVEACNLPIVQGPCRAFIQLWAFDAVKGKCVRFSYGGCKGNGNKFYSQKECKEYCGIPGEA

SEQ ID NO: 122 - protein, 58 aa, Artificial Sequence
  Engineered BPTI (KR15, ME52)
RPDFCLEPPYTGPCRARIIRYFYNAKAGLCQTFVYGGCRAKRNNFKSAEDCERTCGGA

SEQ ID NO: 123 - protein, 59 aa, Artificial Sequence
  Isoaprotinin G-1
  misc_feature (1)..(1): Xaa is Glu or Gln
XRPDFCLEPPYTGPCKARMIRYFYNAKAGLCQPFVYGGCRAKSNNFKSAEDCMRTCGGA

SEQ ID NO: 124 - protein, 58 aa, Artificial Sequence
  Isoaprotinin 2
RPDFCLEPPYTGPCKARIIRYFYNAKAGLCQPFVYGGCRAKRNNFKSSEDCMRTCGGA

SEQ ID NO: 125 - protein, 58 aa, Artificial Sequence
  Isoaprotinin G-2
RPDFCLEPPYTGPCKARMIRYFYNAKAGLCQPFVYGGCRAKRNNFKSAEDCMRTCGGA

SEQ ID NO: 126 - protein, 58 aa, Artificial Sequence; identical to SEQ ID NO: 117
  Isoaprotinin 1

SEQ ID NO: 127 - DNA, 11 bp, Artificial Sequence
  PfMI restriction site
  misc_feature (4)..(8): n is a, c, g or t
ccannnnntgg

SEQ ID NO: 128 - DNA, 15 bp, Artificial Sequence
  XcmI restriction site
  misc_feature (4)..(12): n is a, c, g or t
ccannnnnnnnntgg

SEQ ID NO: 129 - protein, 9 aa, Artificial Sequence
  amino acids 13-21 of EpiNE alpha
PCVAMFQRY

SEQ ID NO: 130 - protein, 6 aa, Artificial Sequence
  amino acids 15-20 of EpiNE-7
VAMFPR

SEQ ID NO: 131 - protein, 4 aa, Artificial Sequence
  amino acids 35-38 of HNE
YGGC

SEQ ID NO: 132 - protein, 9 aa, Artificial Sequence
  amino acids 13-21 of BPTI
PCKARIIRY

SEQ ID NO: 133 - protein, 9 aa, Artificial Sequence
  amino acids 13-21 of EpiNE3
PCVGFFSRY

SEQ ID NO: 134 - protein, 9 aa, Artificial Sequence
  amino acids 13-21 of EpiNE6
PCVGFFQRY

SEQ ID NO: 135 - protein, 9 aa, Artificial Sequence
  amino acids 13-21 of EpiNE7
PCVAMFPRY

SEQ ID NO: 136 - protein, 9 aa, Artificial Sequence
  amino acids 13-21 of EpiNE4
PCVAIFPRY

SEQ ID NO: 137 - protein, 9 aa, Artificial Sequence
  amino acids 13-21 of EpiNE8
PCVAIFKRS

SEQ ID NO: 138 - protein, 58 aa, Artificial Sequence; identical to SEQ ID NO: 7
  EpiNE1

SEQ ID NO: 139 - protein, 9 aa, Artificial Sequence
  amino acids 13-21 of EpiNE5
PCIAFFQRY

SEQ ID NO: 140 - protein, 9 aa, Artificial Sequence
  amino acids 13-21 of EpiNE2
PCIALFKRY

SEQ ID NO: 141 - protein, 58 aa, Artificial sequence
  BITI
RPDFCQLGYSAGPCMGMTSRYFYNGTSMACETFQYGGCMGNGNNFVTEKDCLQTCRGA

SEQ ID NO: 142 - protein, 58 aa, Artificial sequence
  BITI-E7
RPDFCQLGYSAGPCVAMFPRYFYNGTSMACETFQYGGCMGNGNNFVTEKDCLQTCRGA

SEQ ID NO: 143 - protein, 58 aa, Artificial sequence
  BITI-E7-1222
RPDFCQLGYSTGPCVAMFPRYFYNGTSMACETFQYGGCMGNGNNFVTEKDCLQTCRGA

SEQ ID NO: 144 - protein, 58 aa, Artificial sequence
  EpiNE7.6
RPDFCLEPPYTGPCVAMFPRYFYNAKAGLCQTFLYGGCKGKGNNFKSAEDCMRTCGGA

SEQ ID NO: 145 - protein, 58 aa, Artificial sequence
  EpiNE7.8
RPDFCLEPPYTGPCVAMFPRYFYNAKAGLCQTFEYGGCWAKGNNFKSAEDCMRTCGGA

SEQ ID NO: 146 - protein, 58 aa, Artificial sequence
  EpiNE7.11
RPDFCLEPPYTGPCVAMFPRYFYNAKAGLCQTFGYAGCRAKGNNFKSAEDCMRTCGGA

SEQ ID NO: 147 - protein, 58 aa, Artificial sequence
  EpiNE7.7
RPDFCLEPPYTGPCVAMFPRYFYNAKAGLCQTFEYGGCHAEGNNFKSAEDCMRTCGGA

SEQ ID NO: 148 - protein, 58 aa, Artificial sequence
  EpiNE7.4
RPDFCLEPPYTGPCVAMFPRYFYNAKAGLCQTFLYGGCWAQGNNFKSAEDCMRTCGGA

SEQ ID NO: 149 - protein, 58 aa, Artificial sequence
  EpiNE7.5
RPDFCLEPPYTGPCVAMFPRYFYNAKAGLCQTFRYGGCLAEGNNFKSAEDCMRTCGGA

SEQ ID NO: 150 - protein, 58 aa, Artificial sequence
  EpiNE7.10
RPDFCLEPPYTGPCVAMFPRYFYNAKAGLCQTFDYGGCHADGNNFKSAEDCMRTCGGA

SEQ ID NO: 151 - protein, 58 aa, Artificial sequence
  EpiNE7.1
RPDFCLEPPYTGPCVAMFPRYFYNAKAGLCQTFKYGGCLAHGNNFKSAEDCMRTCGGA

SEQ ID NO: 152 - protein, 58 aa, Artificial sequence
  EpiNE7.16
RPDFCLEPPYTGPCVAMFPRYFYNAKAGLCQTFTYGGCWANGNNFKSAEDCMRTCGGA

SEQ ID NO: 153 - protein, 58 aa, Artificial sequence
  EpiNE7.19
RPDFCLEPPYTGPCVAMFPRYFYNAKAGLCQTFNYGGCEGKGNNFKSAEDCMRTCGGA

SEQ ID NO: 154 - protein, 58 aa, Artificial sequence
  EpiNE7.12
RPDFCLEPPYTGPCVAMFPRYFYNAKAGLCQTFQYGGCEGYGNNFKSAEDCMRTCGGA

SEQ ID NO: 155 - protein, 58 aa, Artificial sequence
  EpiNE7.21
RPDFCLEPPYTGPCVAMFPRYFYNAKAGLCQTFHYGGCWGQGNNFKSAEDCMRTCGGA

SEQ ID NO: 156 - protein, 58 aa, Artificial sequence
  EpiNE7.22
RPDFCLEPPYTGPCVAMFPRYFYNAKAGLCQTFHYGGCWGEGNNFKSAEDCMRTCGGA

SEQ ID NO: 157 - protein, 58 aa, Artificial sequence
  EpiNE7.23
RPDFCLEPPYTGPCVAMFPRYFYNAKAGLCQTFKYGGCWGKGNNFKSAEDCMRTCGGA

SEQ ID NO: 158 - protein, 58 aa, Artificial sequence
  EpiNE7.24
RPDFCLEPPYTGPCVAMFPRYFYNAKAGLCQTFKYGGCHGNGNNFKSAEDCMRTCGGA

SEQ ID NO: 159 - protein, 58 aa, Artificial sequence
  EpiNE7.25
RPDFCLEPPYTGPCVAMFPRYFYNAKAGLCQTFPYGGCWAKGNNFKLAEDCMRTCGGA

SEQ ID NO: 160 - protein, 58 aa, Artificial sequence
  EpiNE7.26
RPDFCLEPPYTGPCVAMFPRYFYNAKAGLCQTFKYGGCWGHGNNFKSAEDCMRTCGGA

SEQ ID NO: 161 - protein, 58 aa, Artificial sequence
  EpiNE7.27
RPDFCLEPPYTGPCVAMFPRYFYNAKAGLCQTFNYGGCWGKGNNFKSAEDCMRTCGGA

SEQ ID NO: 162 - protein, 58 aa, Artificial sequence
  EpiNE7.28
RPDFCLEPPYTGPCVAMFPRYFYNAKAGLCQTFTYGGCLGHGNNFKSAEDCMRTCGGA

SEQ ID NO: 163 - protein, 58 aa, Artificial sequence
  EpiNE7.29
RPDFCLEPPYTGPCVAMFPRYFYNAKAGLCQTFTYGGCLGYGNNFKSAEDCMRTCGGA

SEQ ID NO: 164 - protein, 58 aa, Artificial sequence
  EpiNE7.30
RPDFCLEPPYTGPCVAMFPRYFYNAKAGLCQTFKYGGCWAEGNNFKSAEDCMRTCGGA

SEQ ID NO: 165 - protein, 58 aa, Artificial sequence
  EpiNE7.32
RPDFCLEPPYTGPCVAMFPRYFYNAKAGLCQTFGYGGCWGEGNNFKSAEDCMRTCGGA

SEQ ID NO: 166 - protein, 58 aa, Artificial sequence
  EpiNE7.33
RPDFCLEPPYTGPCVAMFPRYFYNAKAGLCQTFEYGGCWANGNNFKSAEDCMRTCGGA

SEQ ID NO: 167 - protein, 58 aa, Artificial sequence
  EpiNE7.36
RPDFCLEPPYTGPCVAMFPRYFYNAKAGLCQTFVYGGCHGDGNNFKSAEDCMRTCGGA

SEQ ID NO: 168 - protein, 58 aa, Artificial sequence
  EpiNE7.37
RPDFCLEPPYTGPCVAMFPRYFYNAKAGLCQTFMYGGCQGKGNNFKSAEDCMRTCGGA

SEQ ID NO: 169 - protein, 58 aa, Artificial sequence
  EpiNE7.38
RPDFCLEPPYTGPCVAMFPRYFYNAKAGLCQTFYYGGCWAKGNNFKSAEDCMRTCGGA

SEQ ID NO: 170 - protein, 58 aa, Artificial sequence
  EpiNE7.39
RPDFCLEPPYTGPCVAMFPRYFYNAKAGLCQTFMYGGCWGDGNNFKSAEDCMRTCGGA

SEQ ID NO: 171 - protein, 58 aa, Artificial sequence
  EpiNE7.40
RPDFCLEPPYTGPCVAMFPRYFYNAKAGLCQTFTYGGCHGNGNNFKSAEDCMRTCGGA

SEQ ID NO: 172 - protein, 58 aa, Artificial sequence
  Synthetic peptide
  MISC_FEATURE (1)..(1): X is R or K
  MISC_FEATURE (2)..(2): X is P or E
  MISC_FEATURE (3)..(3): X is D or A
  MISC_FEATURE (4)..(4): X is F, S, A or E
  MISC_FEATURE (6)..(6): X is L, Q or N
  MISC_FEATURE (7)..(7): X is E or L
  MISC_FEATURE (8)..(8): X is P or G
  MISC_FEATURE (9)..(9): X is P or Y
  MISC_FEATURE (10)..(10): X is Y, S or V
  MISC_FEATURE (11)..(11): X is T, A or R
  MISC_FEATURE (15)..(15): X is V or I
  MISC_FEATURE (16)..(16): X is A or G
  MISC_FEATURE (17)..(17): X is M, F, I or L
  MISC_FEATURE (19)..(19): X is P, S, Q or K
  MISC_FEATURE (21)..(21): X is Y or W
  MISC_FEATURE (22)..(22): X is F or A
  MISC_FEATURE (23)..(23): X is Y or F
  MISC_FEATURE (24)..(24): X is N or D
  MISC_FEATURE (25)..(25): X is A or G
  MISC_FEATURE (26)..(26): X is K, T or V
  MISC_FEATURE (27)..(27): X is A, S or K
  MISC_FEATURE (28)..(28): X is G or M
  MISC_FEATURE (29)..(29): X is L, A or K
  MISC_FEATURE (31)..(31): X is Q, E or V
  MISC_FEATURE (32)..(32): X is T or L
  MISC_FEATURE (34)..(34): X is V, Q, P, L, E, G, R, D, K, T, N, H, M or Y
  MISC_FEATURE (39)..(39): X is M, Q, K, W,  R, H, L or E
  MISC_FEATURE (40)..(40): X is G or A
  MISC_FEATURE (41)..(41): X is N, K, E, Q, D, H or Y
  MISC_FEATURE (44)..(44): X is N or K
  MISC_FEATURE (46)..(46): X is K, V or Y
  MISC_FEATURE (47)..(47): X is S or T
  MISC_FEATURE (48)..(48): X is A or E
  MISC_FEATURE (49)..(49): X is E or K
  MISC_FEATURE (50)..(50): X is D or E
  MISC_FEATURE (52)..(52): X is M, L or R
  MISC_FEATURE (53)..(53): X is R, Q or E
  MISC_FEATURE (54)..(54): X is T or Y
  MISC_FEATURE (56)..(56): X is G or R
  MISC_FEATURE (57)..(57): X is G or V
  MISC_FEATURE (58)..(58): X is A or P
XXXXCXXXXXXGPCXXXFXRXXXXXXXXXCXXFXYGGCXXXGNXFXXXXXCXXXCXXX

SEQ ID NO: 173 - protein, 58 aa, Artificial sequence; identical to SEQ ID NO: 9
  Synthetic peptide

(19) United States
(12) Patent Application Publication
(10) Pub. No.: US 2006/0134087 A1
(43) Pub. Date: Jun. 22, 2006

(54) ITI-D1 Kunitz domain mutants as hNE inhibitors

(75) Inventors: Arthur Charles Ley, Newton, MA (US); Sonia Kosow Guterman, Belmont, MA (US); William Markland, Milford, MA (US); Rachel Baribault Kent, Boxborough, MA (US); Bruce Lindsay Roberts, Milford, MA (US); Robert Charles Ladner, Ijamsville, MD (US)

Correspondence Address: WOLF GREENFIELD & SACKS, PC;FEDERAL RESERVE PLAZA, 600 ATLANTIC AVENUE, BOSTON, MA, 02210-2206, US

(73) Assignee: Dyax Corp., Cambridge, MA (US)

(21) Appl. No.: 11/253,176

(22) Filed: Oct. 18, 2005

Related U.S. Application Data
(62) Division of application No. 10/038,722, filed on Jan. 8, 2002.
Application No. 10/038,722 is a continuation of application No. 08/849,406, filed on Jul. 21, 1999, now abandoned.
Application No. 08/849,406 is a continuation-in-part of application No. 08/358,160, filed on Dec. 16, 1994, now Pat. No. 5,663,143.
Application No. 08/358,160 is a continuation-in-part of application No. 08/133,031, filed on Oct. 13, 1993, now abandoned.
Application No. 08/133,031 is a division of application No. 07/664,989, filed on Mar. 1, 1991, now Pat. No. 5,223,409.
Application No. 07/664,989 is a continuation-in-part of application No. 07/240,160, filed on Sep. 2, 1988, now abandoned.

(51) Int. Cl. A61K 38/54; C12N 9/99

(52) U.S. Cl. 424/94.2; 435/184

(57) ABSTRACT

Mutants of Kunitz domain 1 (ITI-D1) of human inter-α-trypsin inhibitor (ITI), are useful as inhibitors of human neutrophil elastase. Mutants characterized by one or more of the following substitutions (numbered to correspond to bovine pancreatic trypsin inhibitor, the archetypal Kunitz domain) are of particular interest: (a) Val15 or Ile15, (b) Ala16, (c) Phe18, (d) Pro19, (e) Arg1, (f) Pro2, and/or (g) Phe4.

### CROSS-REFERENCE TO RELATED APPLICATIONS

This application is a divisional of application Ser. No. 10/038,722, filed Jan. 8, 2002, now allowed which is a continuation of application Ser. No. 08/849,406, filed Jul. 21, 1999, now abandoned, which is a National Stage of International Application Number PCT/US95/16349, filed Dec. 15, 1995, which is a continuation-in-part of Issued U.S. Pat. No. 5,663,143, filed Dec. 16, 1994, which is a continuation-in-part of application Ser. No. 08/133,031, filed Feb. 28, 1992 (abandoned), the entire disclosures of which are incorporated herein by reference.

The following applications are incorporated herein by reference. Application Ser. No. 08/133,031, filed Feb. 28, 1992 (abandoned), which is a National Stage of International application number PCT/US92/01501, filed Feb. 28, 1992, which is a divisional of Issued U.S. Pat. No. 5,223,409, filed Mar. 1, 1991, which is a continuation-in-part of application Ser. No. 07/240,160, filed Sep. 2, 1988 (abandoned).

The following related and commonly-owned applications are also incorporated by reference:

Robert Charles Ladner, Sonia Kosow Guterman, Rachel Baribault Kent, and Arthur Charles Ley are named as joint inventors on U.S. Ser. No. 07/293,980, filed Jan. 8, 1989, and entitled GENERATION AND SELECTION OF NOVEL DNA-BINDING PROTEINS AND POLYPEPTIDES. This application has been assigned to Protein Engineering Corporation.

Robert Charles Ladner, Sonia Kosow Guterman, and Bruce Lindsay Roberts are named as a joint inventors on a U.S. Ser. No. 07/470,651 filed 26 Jan. 1990 (now abandoned), entitled “PRODUCTION OF NOVEL SEQUENCE-SPECIFIC DNA-ALTERING ENZYMES”, likewise assigned to Protein Engineering Corp.

La dner, Guterman, Kent, Ley, and Markland, Ser. No. 07/558,011 is also assigned to Protein Engineering Corporation.

Ladner filed an application on May 17, 1991, Ser. No. 07/715,834 that is hereby incorporated by reference.

### SEQUENCE LISTING

The entire contents of the compact disc containing the Sequence Listing identified as “D0617.70005US03 Sequence Listing”, created on Oct. 17, 2005, containing 141 KB is herein incorporated by reference.

### BACKGROUND OF THE INVENTION

### Field of the Invention

This invention relates to novel proteins that inhibit human neutrophil elastase (hNE). A large fraction of the sequence of each of these proteins is identical to a known human protein which has very little or no inhibitory activity with respect to hNE.

Information Disclosure Statement

1. hNE, its Natural Inhibitors, and Pathologies

Human Neutrophil Elastase (hNE, also known as Human Leukocyte Elastase (hLE); EC 3.4.21.11) is a 29 Kd protease with a wide spectrum of activity against extracellular matrix components (CAMP82, CAMP88, MCWH89). The enzyme is one of the major neutral proteases of the azurophil granules of polymorphonuclear leucocytes and is involved in the elimination of pathogens and in connective tissue restructuring (TRAV88). In cases of hereditary reduction of the circulating α-1-protease inhibitor (API, formerly known as α1 antitrypsin), the principal systemic physiological inhibitor of hNE (HEID86), or the inactivation of API by oxidation (“smoker's emphysema”), extensive destruction of lung tissue may result from uncontrolled elastolytic activity of hNE (CANT89). Several human respiratory disorders, including cystic fibrosis and emphysema, are characterized by an increased neutrophil burden on the epithelial surface of the lungs (SNID91, MCEL91, GOLD86) and hNE release by neutrophils is implicated in the progress of these disorders (MCEL91, WEIS89). A preliminary study of aerosol administration of API to cystic fibrosis patients indicates that such treatment can be effective both in prevention of respiratory tissue damage and in augmentation of host antimicrobial defenses (MCEL91).

API presents some practical problems to large-scale routine use as a pulmonary anti-elastolytic agent. These include the relatively large size of the molecule (394 residues, 51 k Dalton), the lack of intramolecular stabilizing disulfide bridges, and specific post translational modifications of the protein by glycosylation at three sites. Perhaps of even greater importance is the sensitivity of API to oxidation, such as those released by activated neutrophils. Hence a small stable nontoxic highly efficacious inhibitor of hNE would be of great therapeutic value.

2. Proteinaceous Serine Protease Inhibitors. A large number of proteins act as serine protease inhibitors by serving as a highly specific, limited proteolysis substrate for their target enzymes. In many cases, the reactive site peptide bond (“scissile bond”) is encompassed in at least one disulfide loop, which insures that during conversion of virgin to modified inhibitor the two peptide chains cannot dissociate.

A special nomenclature has evolved for describing the active site of the inhibitor. Starting at the residue on the amino side of the scissile bond, and moving away from the bond, residues are named P1, P2, P3, etc. (SCHE67). Residues that follow the scissile bond are called P1′, P2′, P3′, etc. It has been found that the main chain of protein inhibitors having very different overall structure are highly similar in the region between P3 and P3′ with especially high similarity for P2, P₁ and P1′ (LASK80 and works cited therein). It is generally accepted that each serine protease has sites S1, S2, etc. that receive the side groups of residues P1, P2, etc. of the substrate or inhibitor and sites S1′, S2′, etc. that receive the side groups of P1′, P2′, etc. of the substrate or inhibitor (SCHE67). It is the interactions between the S sites and the P side groups that give the protease specificity with respect to substrates and the inhibitors specificity with respect to proteases.

The serine protease inhibitors have been grouped into families according to both sequence similarity and the topological relationship of their active site and disulfide loops. The families include the bovine pancreatic trypsin inhibitor (Kunitz), pancreatic secretory trypsin inhibitor (Kazal), the Bowman-Birk inhibitor, and soybean trypsin inhibitor (Kunitz) families. Some inhibitors have several reactive sites on a single polypeptide chains, and these distinct domains may have different sequences, specificities, and even topologies.

One of the more unusual characteristics of these inhibitors is their ability to retain some form of inhibitory activity even after replacement of the P1 residue. It has further been found that substituting amino acids in the P₅ to P₅′ region, and more particularly the P3 to P3′ region, can greatly influence the specificity of an inhibitor. LASK80 suggested that among the BPTI (Kunitz) family, inhibitors with P1 Lys and Arg tend to inhibit trypsin, those with P1=Tyr, Phe, Trp, Leu and Met tend to inhibit chymotrypsin, and those with P1=Ala or Ser are likely to inhibit elastase. Among the Kazal inhibitors, they continue, inhibitors with P1=Leu or Met are strong inhibitors of elastase, and in the Bowman-Kirk family elastase is inhibited with P1 Ala, but not with P1 Leu.

“Kunitz” Domain Proteinase Inhibitors. Bovine pancreatic trypsin inhibitor (BPTI, a.k.a. aprotonin) is a 58 a.a. serine proteinase inhibitor of the BPTI (Kunitz) domain (KuDom) family. Under the tradename TRASYLOL, it is used for countering the effects of trypsin released during pancreatitis. Not only is its 58 amino acid sequence known, the 3D structure of BPTI has been determined at high resolution by X-ray diffraction (HUBE77, MARQ83, WLOD84, WLOD87a, WLOD87b), neutron diffraction (WLOD84), and by NMR (WAGN87). One of the X-ray structures is deposited in the Brookhaven Protein Data Bank as “6PTI” [sic]. The 3D structure of various BPTI homologues (EIGE90, HYNE90) are also known. At least sixty homologues have been reported; the sequences of 39 homologues are given in Table 5. The known human homologues include domains of Lipoprotein Associated Coagulation Inhibitor (LACI) (WUNT88, GIRA89), Inter-α-Trypsin Inhibitor (ALBR83a, ALBR83b, DIAR90, ENGH89, TRIB86, GEBH86, GEBH90, KAUM86, ODOM90, SALI90), and the Alzheimer beta-Amyloid Precursor Protein. Circularized BPTI and circularly permuted BPTI have binding properties similar to BPTI (GOLD83). Some proteins homologous to BPTI have more or fewer residues at either terminus.

In BPTI, the P1 residue is at position 15. Tschesche et al. (TSCH87) reported on the binding of several BPTI P1 derivatives to various proteases:

TABLE 1
Dissociation constants for BPTI P1 derivatives, Molar.

| Residue | Trypsin | Chymotrypsin | Elastase | Elastase |
|---|---|---|---|---|
| #15 | (bovine | (bovine | (porcine | (human |
| P1 | pancreas) | pancreas) | pancreas) | leukocytes) |
| lysine | 6.0 · 10⁻¹⁴ | 9.0 · 10⁻⁹ | − | 3.5 · 10⁻⁶(WT) |
| glycine | − | − | + | 7.0 · 10⁻⁹ |
| alanine | + | − | 2.8 · 10⁻⁸ | 2.5 · 10⁻⁹ |
| valine | − | − | 5.7 · 10⁻⁸ | 1.1 · 10⁻¹⁰ |
| leucine | − | − | 1.9 · 10⁻⁸ | 2.9 · 10⁻⁹ |

From the report of Tschesche et al. we infer that molecular pairs marked “+” have K_{d}s≧3.5·10⁻⁶ M and that molecular pairs marked “−” have K_{d}s>>3.5·10⁻⁶ M. It is apparent that wild-type BPTI has only modest affinity for hNE, however, mutants of BPTI with higher affinity are known. While not shown in the Table, BPTI does not significantly bind hCG. However, Brinkmann and Tschesche (BRIN90) made a triple mutant of BPTI (viz. K15F, R17F, M52E) that has a Kᵢ with respect to hCG of 5.0×10⁻⁷ M.

3. ITI Domain 1 and ITI Domain 2 as an Initial Protein Binding Domains (IPBD)

Many mammalian species have a protein in their plasma that can be identified, by sequence homology and similarity of physical and chemical properties, as inter-α-trypsin inhibitor (ITI), a large (Mᵣ ca 240,000) circulating protease inhibitor (for recent reviews see ODOM90, SALI90, GEBH90, GEBH86). The sequence of human ITI is shown in Table 28. The intact inhibitor is a glycoprotein and is currently believed to consist of three glycosylated subunits that interact through a strong glycosaminoglycan linkage (ODOM90, SALI90, ENGH89, SELL87). The anti-trypsin activity of ITI is located on the smallest subunit (ITI light chain, unglycosylated Mᵣ ca 15,000) which is identical in amino acid sequence to an acid stable inhibitor found in urine (UTI) and serum (STI) (GEBH86, GEBH90). The amino-acid sequence of the ITI light chain is shown in Table 28. The mature light chain consists of a 21 residue N-terminal sequence, glycosylated at Ser₁₀, followed by two tandem Kunitz-type domains the first of which is glycosylated at Asn₄₅ (ODOM90). In the human protein, the second Kunitz-type domain has been shown to inhibit trypsin, chymotrypsin, and plasmin (ALBR83a, ALBR83b, SELL87, SWAI88). The first domain lacks these activities but has been reported to inhibit leukocyte elastase (≈1 μM>Kᵢ>≈1 nM) (ALBR83a,b, ODOM90). cDNA encoding the ITI light chain also codes for α-1-microglobulin (TRAB86, KAUM86, DIAR90); the proteins are separated post-translationally by proteolysis.

The two Kunitz domains of the ITI light chain (ITI-D1 and ITI-D2) possesses a number of characteristics that make them useful as Initial Potential Binding Domains (IPBDs). ITI-D1 comprises at least residues 26 to 76 of the UTI sequence shown in FIG. 1 of GEBH86. The Kunitz domain could be thought of as comprising residues from as early as residue 22 to as far as residue 79. Residues 22 through 79 constitute a 58-amino-acid domain having the same length as bovine pancreatic trypsin inhibitor (BPTI) and having the cysteines aligned. ITI-D2 comprises at least residues 82 through 132; residues as early as 78 and as later as 135 could be included to give domains closer to the classical 58-amino-acid length. As the space between the last cysteine of ITI-D1 (residue 76 of ITI light chain) and the first cysteine of ITI-D2 (residue 82 of ITI light chain) is only 5 residues, one can not assign 58 amino acids to each domain without some overlap. Unless otherwise stated, herein, we have taken the second domain to begin at residue 78 of the ITI light chain. Each of the domains are highly homologous to both BPTI and the EpiNE series of proteins described in U.S. Pat. No. 5,223,409. Although x-ray structures of the isolated domains ITI-D1 and ITI-D2 are not available, crystallographic studies of the related Kunitz-type domain isolated from the Alzheimer's amyloid β-protein (AAβP) precursor show that this polypeptide assumes a 3D structure almost identical to that of BPTI (HYNE90).

The three-dimensional structure of α-dendrotoxin from green mamba venom has been determined (SKAR92) and the structure is highly similar to that of BPTI. The author states, “Although the main-chain fold of α-DTX is similar to that of homologous bovine pancreatic trypsin inhibitor (BPTI), there are significant differences involving segments of the polypeptide chain close to the ‘antiprotease site’ of BPTI. Comparison of the structure of α-DTX with the existing models of BPTI and its complexes with trypsin and kallikrein reveals structural differences that explain the inability of α-DTX to inhibit trypsin and chymotrypsin.”

The structure of the black mamba K venom has been determined by NMR spectroscopy and has a 3D structure that is highly similar to that of BPTI despite 32 amino-acid sequence differences between residues 5 and 55 (the first and last cysteines)(BERN93). “The solution structure of Toxin K is very similar to the solution structure of the basic pancreatic trypsin inhibitor (BPTI) and the X-ray crystal structure of the α-dendrotoxin from Dendroaspis angusticeps (α-DTX), with r.m.s.d. values of 1.31 Å and 0.92 Å, respectively, for the backbone atoms of residues 2 to 56. Some local structural differences between Toxin K and BPTI are directly related to the fact that intermolecular interactions with two of the four internal molecules of hydration water in BPTI are replaced by intramolecular hydrogen bonds in Toxin K.” Thus, it is likely that the solution 3D structure of either of the isolated ITI-D1 domain or of the isolated ITI-D2 domain will be highly similar to the structures of BPTI, AAβP, and black mamba K venom. In this case, the advantages described previously for use of BPTI as an IPBD apply to ITI-D1 and to ITI-D2. ITI-D1 and ITI-D2 provide additional advantages as an IPBD for the development of specific anti-elastase inhibitory activity. First, the ITI-D1 domain has been reported to inhibit both leukocyte elastase (ALBR83a,b, ODOM90) and Cathepsin-G (SWAI88, ODOM90); activities which BPTI lacks. Second, ITI-D1 lacks affinity for the related serine proteases trypsin, chymotrypsin, and plasmin (ALBR83a,b, SWAI88), an advantage for the development of specificity in inhibition. ITI-D2 has the advantage of not being glycosylated. Additionally, ITI-D1 and ITI-D2 are human-derived polypeptides so that derivatives are anticipated to show minimal antigenicity in clinical applications.

4. Secretion of Heterologous Proteins from Pichia pastoris

Others have produced a number of proteins in the yeast Pichia pastoris. For example, Vedvick et al. (VEDV91) and Wagner et al. (WAGN92) produced aprotinin from the alcohol oxidase promoter with induction by methanol as a secreted protein in the culture medium (CM) at ≈1 mg/mL. Gregg et al. (GREG93) have reviewed production of a number of proteins in P. pastoris. Table 1 of GREG93 shows proteins that have been produced in P. pastoris and the yields.

5. Recombinant Production of Kunitz Domains:

Aprotinin has been made via recombinant-DNA technology (AUER87, AUER88, AUER89, AUER90, BRIN90, BRIN91, ALTM91).

6. Construction Methods:

Unless otherwise stated, genetic constructions and other manipulations are carries out by standard methods, such as found in standard references (e.g. AUSU87 and SAMB89).

No admission is made that any cited reference is prior art or pertinent prior art, and the dates given are those appearing on the reference and may not be identical to the actual publication date. The descriptions of the teachings of any cited reference are based on our present reading thereof, and we reserve the right to revise the description if an error comes to our attention, and to challenge whether the description accurately reflects the actual work reported. We reserve the right to challenge the interpretation of cited works, particularly in light of new or contradictory evidence.

### SUMMARY OF THE INVENTION

The present invention describes a series of small potent proteinaceous inhibitors of human neutrophil elastase (hNE). One group of inhibitors is derived from a Kunitz-type inhibitory domain found in a protein of human origin, namely, the light chain of human Inter-α-trypsin inhibitor (ITI) which contains domains designated ITI-D1 and ITI-D2. The present invention discloses variants of ITI-D1 and ITI-D2 that have very high affinity for hNE. The present invention comprises modifications to the ITI-D2 sequence that facilitate its production in the yeast Pichia pastoris and that are highly potent inhibitors of hNE. The invention also relates to methods of transferring segments of sequence from one Kunitz domain to another and to methods of production.

The invention is presented as a series of examples that describe design, production, and testing of actual inhibitors and additional examples describing how other inhibitors could be discovered. The invention relates to proteins that inhibit human neutrophil elastase (hNE) with high affinity.

TABLE 2
NOMENCLATURE and ABBREVIATIONS

| Term | Meaning |
|---|---|
| x::y | Fusion of gene x to gene y in frame. |
| X::Y | Fusion protein expressed from x::y fusion gene. |
| μM | Micromolar, 10⁻⁶ molar. |
| nM | Namomolar, 10⁻⁹ molar. |
| pM | Picomolar, 10⁻¹² molar. |

Single-letter amino-acid codes:

| | | | |
|---|---|---|---|
| A: Ala | C: Cys | D: Asp | E: Glu |
| F: Phe | G: Gly | H: His | I: Ile |
| K: Lys | L: Leu | M: Met | N: Asn |
| P: Pro | Q: Gln | R: Arg | S: Ser |
| T: Thr | V: Val | W: Trp | Y: Tyr |

### DETAILED DESCRIPTION OF THE PREFERRED EMBODIMENTS OF THE INVENTION

A protein sequence can be called an “aprotinin-like Kunitz domain” if it contains a sequence that when aligned to minimize mismatches, can be aligned, with four or fewer mismatches, to the pattern: Cys-(Xaa)₆-Gly-Xaa-Cys-(Xaa)₈-[Tyr|Phe]-(Xaa)₆-Cys-(Xaa)₂-Phe-Xaa-[Tyr|Trp|Phe]-Xaa-Gly-Cys-(Xaa)₄-[Asn|Gly]-Xaa-[Phe|Tyr]-(Xaa)₅-Cys-(Xaa)₃-Cys (SEQ ID NO:86), where bracketed amino acids separated by a | symbol are alternative amino acids for a single position. For example, [Tyr|Phe] indicates that at that position, the amino acid may be either Tyr or Phe. The symbol Xaa denotes that at that position, any amino acid may be used. For the above test, an insertion or deletion counts as one mismatch.

In aprotonin, the cysteines are numbered 5, 14, 30, 38, 51, and 55 and are joined by disulfides 5-to-55, 14-to-38, and 30-to-51. Residue 15 is called the P1 residue (SCHE67); residues toward the amino terminus are called P2(residue 14), P3(residue 13), etc. Residue 16 is called P1′, 17 is P2′, 18 is P3′, etc.

There are many homologues of aprotonin, which differ from it at one or more positions but retain the fundamental structure defined above. For a given list of homologues, it is possible to tabulate the frequency of occurrence of each amino acid at each ambiguous position. (The sequence having the most prevalent amino acid at each ambiguous position is listed as “Consensus Kunitz Domain” in Table 10).

A “human aprotonin-like Kunitz domain” is an aprotonin-like Kunitz domain which is found in nature in a human protein. Human aprotonin-like Kunitz domains include, but are not limited to, ITI-D1, ITI-D2, App-1, TFPI2-D1, TFPI2-D2, TFPI2-D3, LACI-D1, LACI-D2, LACI-D3, A3 collagen, and the HKI B9 domain. In this list, D1, D2, etc., denote the first, second, etc. domain of the indicated multidomain protein.

“Weak”, “Moderate”, “Strong” and “Very Strong” binding to and inhibition of hNE are defined in accordance with Table 8. Preferably, the proteins of the present invention have a Ki of less than 1000 pM (i.e., are “strong” inhibitors), more preferably less than 50 pM, most preferably less than 10 pM (i.e., are “very strong” inhibitors).

For purposes of the present invention, an aprotonin-like Kunitz domain may be divided into ten segments, based on the consensus sequence and the location of the catalytic site. Using the amino acid numbering scheme of aprotonin, these segments are as follows (see Table 10):

1: 1-4 (residues before first Cys)

2: 5-9 (first Cys and subsequent residues before P6)

3: 10-13 (P6 to P3)

4: 14 (second Cys; P2)

5: 15-21 (P1, and P1′ to P6′)

6: 22-30 (after P6 and up to and incl. third Cys.)

7: 31-36 (after third Cys and up to consensus Gly-Cys)

8: 37-38 (consensus Gly-Cys)

9: 39-42 (residues after Gly-Cys and before consensus [Asn|Gly]

10: 43-55 (up to last Cys)(also includes residues after last Cys, if any)

It will be appreciated that in those aprotonin-like Kunitz domains that differ from aprotonin by one or more amino acid insertions or deletions, or which have a different number of amino acids before the first cysteine or after the last cysteine, the actual amino acid position may differ from that given above. It is applicant's intent that these domains be numbered so as to correspond to the aligned aprotonin sequence, e.g., the first cysteine of the domain is numbered amino acid 5, for the purpose of segment identification. Note that segment 1, while a part of aprotonin, is not a part of the formal definition of an aprotonin-like Kunitz domain, and therefore it is not required that the proteins of the present invention include a sequence corresponding to segment 1. Similarly, part of segment 10 (after the last Cys) is not a required part of the domain.

A “humanized inhibitor” is one in which at least one of segments 3, 5, 7 and 9 differs by at least one nonconservative modification from the most similar (based on amino acid identities) human aprotonin-like Kunitz domain, at least one of segments 2, 6, and 10 (considered up to the last Cys) is identical, or differs only by conservative modifications, from said most similar human aprotonin-like Kunitz domain, and which is not identical to any naturally occurring nonhuman aprotonin-like Kunitz domain. (Note that segment 1 is ignored in making this determination since it is outside the sequence used to define a domain, and segments 4 and 8 are ignored because they are required by the definition of an aprotonin-like Kunitz domain.)

The proteins of the present invention are preferably humanized strong or very strong hNE inhibitors. It should be noted that the human aprotonin-like Kunitz domains thus far identified are merely weak hNE inhibitors.

For the purpose of the appended claims, an aprotonin-like Kunitz domain is “substantially homologous” to a reference domain if, over the critical region (aprotonin residues 5-55) set forth above, it is at least at least 50% identical in amino acid sequence to the corresponding sequence of or within the reference domain, and all divergences take the form of conservative and/or semi-conservative modifications.

Proteins of the present invention include those comprising a Kunitz domain that is substantially homologous to the reference proteins EPI-HNE-3, EPI-HNE-4, DPI.1.1, DPI.1.2, DPI.1.3, DPI.2.1, DPI.2.2, DPI.2.3, DPI.3.1, DPI.3.2, DPI.3.3, DPI.4.1, DPI.4.2, DPI.4.3, DPI.5.1, DPI.5.2, DPI.5.3, DPI.6.1, DPI.6.2, DPI.6.3, DPI.6.4, DPI.6.5, DPI.6.6, DPI.6.7, DPI.7.1, DPI.7.2, DPI.7.3, DPI.7.4, DPI.7.5, DPI.8.1, DPI.8.2, DPI.8.3, DPI.9.1, DPI.9.2, or DPI.9.3, as defined in Table 10. Homologues of EPI-HNE-3 and EPI-HNE-4 are especially preferred.

Preferably, the hNE-binding domains of the proteins of the present invention are at least 80% identical, more preferably, at least 90% identical, in amino acid sequence to the corresponding reference sequence. Most preferably, the number of mismatches is zero, one, two, three, four or five. Desirably, the hNE-binding domains diverge from the reference domain solely by one or more conservative modifications.

“Conservative modifications” are defined as:
a) conservative substitutions of amino acids as hereafter defined, and
b) single or multiple insertions or deletions of amino acids at the termini, at interdomain boundaries, in loops or in other segments of relatively high mobility (as indicated, for example, by high temperature factors or lack of resolution in X-ray diffraction, neutron diffraction, or NMR). Preferably, except at the termini, no more than about five amino acids are inserted or deleted at a particular locus, and the modifications are outside regions known to contain binding sites important to activity.

“Conservative substitutions” are herein defined as exchanges within on of the following five groups:
I. Small aliphatic, nonpolar or slightly polar residues: [Ala, Ser, Thr, (Pro, Gly)],
II. Acidic amino acids and their amides: [Asp, Glu, Asn, Gln],
III. Polar, positively charged residues: [His, Lys, Arg],
IV. Aliphatic nonpolar residues: [Met, Leu, Ile, Val, (Cys)], and
V. Large, aromatic residues: [Phe, Tyr, Trp]

Residues Pro, Gly, and Cys are parenthesized because they have special conformational roles. Cys often participates in disulfide bonds; when not so doing, it is highly hydrophobic. Gly imparts flexibility to the chain; it is often described as a “helix breaker” although many α helices contain Gly. Pro imparts rigidity to the chain and is also described as a “helix breaker”. Although Pro is most often found in turns, Pro is also found in helices and sheets. These residues may be essential at certain positions and substitutable elsewhere.

Semi-Conservative Modifications” are defined herein as transpositions of adjacent amino acids (or their conservative replacements), and semi-conservative substitutions. “Semi-conservative substitutions” are defined to be exchanges between two of groups (I)-(V) above which are limited either to the supergroup consisting of (I), (II), and (III) or to the supergroup consisting of (IV) and (V). For the purpose of this definition, however, glycine and alanine are considered to be members of both supergroups.

“Non-conservative modifications” are modifications which are neither conservative nor semi-conservative.

Preferred proteins of the present invention are further characterized by one of more of the preferred, highly preferred, or most preferred mutations set forth in Table 41.

Preferably, the proteins of the present invention have hNE-inhibitory domains which are not only substantially homologous to a reference domain, but also qualify as humanized inhibitors.

Claim 1 of PCT/US92/01501 refers to proteins denoted EpiNEalpha, EpiNE1, EpiNE2, EpiNE3, EpiNE4, EpiNE5, EpiNE6, EpiNE7, and EpiNE8. Claim 3 refers to proteins denoted ITI-E7, BITI-E7, BITI-E&-1222, AMINO1, AMINO2, MUTP1, BITI-E7-141, MUTT26A, MUTQE, and MUT1619. (With the exception of EpiNEalpha, the sequences of all of these domains appears in Table 10). Claims 4-6 related to inhibitors which are homologous to, but not identical with, the aforementioned inhibitors. These homologous inhibitors could differ from the lead inhibitors by one or more class A substitutions (claim 4), one or more class A or B substitutions (claim 5), or one or more class A, B or C substitutions (claim 6). Class A, B and C substitutions were defined in Table 65 of PCT/US92/01501. For convenience, Table 65 has been duplicated in this specification (Table 9).

The meaning of classes A, B and C were as follows: A, no major effect expected if molecular charge stays in range −1 to +1; B, major effects not expected, but more likely than with A; and C, residue in binding interface, any change must be tested. Each residue position was assigned an A, B, C or X rating; X meant no substitution allowed. At the non-X positions, allowed substitutions were noted.

In one series of embodiments, the present invention is directed to HNE inhibitors as disclosed in Ser. No. 08/133,031 (previously incorporated by reference), which is the U.S. national stage of PCT/US92/01501.

The invention disclosed in Ser. No. 08/133,031 relates to muteins of BPTI, ITI-D1 and other Kunitz domain-type inhibitors which have a high affinity for elastase. Some of the described inhibitors are derived from BPTI and some from ITI-D1. However, hybrids of the identified muteins and other Kunitz domain-type inhibitors could be constructed.

For the purpose of simultaneously assessing the affinity of a large number of different BPTI and ITI-D1 muteins, DNA sequences encoding the BPTI or ITI-D1 was incorporated into the genome of the bacteriophage M13. The KuDom is displayed on the surface of M13 as an amino-terminal fusion with the gene III coat protein. Alterations in the KuDom amino acid sequence were introduced. Each pure population of phage displaying a particular KuDom was characterized with regard to its interactions with immobilized hNE or hCG. Based on comparison to the pH elution profiles of phage displaying other KuDoms of known affinities for the particular protease, mutant KuDoms having high affinity for the target proteases were identified. Subsequently, the sequences of these mutant KuDoms were determined (typically by sequencing the corresponding DNA sequence).

Certain aprotonin-like protease inhibitors were shown to have a high affinity for HNE (≈10¹²/M). These 58 amino acid polypeptides were biologically selected from a library of aprotinin mutants produced through synthetic diversity. Positions P1, P1′, P2′, P3′, and P4′ were varied. At P1, only VAL and ILE were selected, although LEU, PHE, and MET were allowed by the synthetic conditions. At P1′, ALA and GLY were allowed and both were found in proteins having high affinity. (While not explored in the library, many Kazal family inhibitors of serine proteases have glutamic or aspartic acid at P1′.) All selected proteins contained either PHE or MET at P2′; LEU, ILE, and VAL, which are amino acids with branched aliphatic side groups, were in the library but apparently hinder binding to HNE. Surprisingly, position P3′ of all proteins selected for high affinity for HNE have phenylalanine. No one had suggested that P3′ was a crucial position for determining specificity relative to HNE. At P4′, SER, PRO, THR, LYS, and GLN were allowed; all of these except THR were observed. PRO and SER are found in the derivatives having the highest affinity.

In Ser. No. 08/133,031, Table 61 showed the variability of 39 naturally-occurring Kunitz domains. All these proteins have 51 residues in the region C₅ through C₅₅; the total number of residues varies due to the proteins having more or fewer residues at the termini. Table 62 list the names of the proteins that are included in Table 61. Table 64 cites works where these sequences are recorded. Table 63 shows a histogram of how many loci show a particular variability vs. the variability. “Core” refers to residues from 5 to 55 that show greater sequence and structural similarity than do residues outside the core.

At ten positions a single amino-acid type is observed in all 42 cases, these are C₅, G₁₂, C₁₄, C₃₀, F₃₃, G₃₇, C₃₈, N₄₃, C₅₁, and C₅₅. Although there are reports that each of these positions may be substituted without complete loss of structure, only G₁₂, C₁₄, G₃₇, and C₃₈ are close enough to the binding interface to offer any incentive to make changes. G₁₂ is in a conformation that only glycine can attain; this residue is best left as is. Marks et al. (MARK87) replaced both C₁₄ and C₃₈ with either two alanines or two threonines. The C₁₄/C₃₈ cystine bridge that Marks et al. removed is the one very close to the scissile bond in BPTI; surprisingly, both mutant molecules functioned as trypsin inhibitors. Both BPTI(C14A,C38A) and BPTI(C14T,C38T) are stable and inhibit trypsin. Altering these residues might give rise to a useful inhibitor that retains a useful stability, and the phage-display of a variegated population is the best way to obtain and test mutants that embody alterations at either 14 or 38. Only if the C₁₄/C₃₈ disulfide is removed, would the strict conservation of G₃₇ be removed.

At seven positions (viz. 23, 35, 36, 40, 41, 45, and 47) only two amino-acid types have been found. At position 23 only Y and F are observed; the para position of the phenyl ring is solvent accessible and far from the binding site. Changes here are likely to exert subtle influences on binding and are not a high priority for variegation. Similarly, 35 has only the aromatic residues Y and W; phenylalanine would probably function well here. At 36, glycine predominates while serine is also seen. Other amino acids, especially {N, D, A, R}, should be allowed and would likely affect binding properties. Position 40 has only G or A; structural models suggest that other amino acids would be tolerated, particularly those in the set {S, D, N, E, K, R, L, M, Q, and T}. Position 40 is close enough to the binding site that alteration here might affect binding. At 41, only N, and K have been seen, but any amino acid, other than proline, should be allowed. The side group is exposed, so hydrophilic side groups are preferred, especially {D, S, T, E, R, Q, and A}. This residue is far enough from the binding site that changes here are not expected to have big effects on binding. At 45, F is highly preferred, but Y is observed once. As one edge of the phenyl ring is exposed, substitution of other aromatics (W or H) is likely to make molecules of similar structure, though it is difficult to predict how the stability will be affected. Aliphatics such as leucine or methionine (not having branched C_{β}s) might also work here. At 47, only S and T have been seen, but other amino acids, especially {N, D, G, and A}, should give stable proteins.

At one position (44), only three amino-acid types have been observed. Here, asparagine predominates and may form internal hydrogen bonds. Other amino acids should be allowed, excepting perhaps proline.

At the remaining 40 positions, four or more amino acids have been observed; at 28 positions, eight or more amino-acid types are seen. Position 25 exhibits 13 different types and 5 positions (1, 6, 17, 26, and 34) exhibit 12 types. Proline (the most rigid amino acid) has been observed at fourteen positions: 1, 2, 8, 9, 11, 13, 19, 25, 32, 34, 39, 49, 57, and 58. The φ, ψ angles of BPTI (CREI84, Table 6-3, p. 222) indicate that proline should be allowed at positions 1, 2, 3, 7, 8, 9, 11, 13, 16, 19, 23, 25, 26, 32, 35, 36, 40, 42, 43, 48, 49, 50, 52, 53, 54, 56, and 58. Proline occurs at four positions (34, 39, 57, and 58) where the BPTI φ, ψ angles indicate that it should be unacceptable. We conclude that the main chain rearranges locally in these cases.

Based on these data and excluding the six cysteines, we judge that the KuDom structure will allow those substitutions shown in Table 9. The class indicates whether the substitutions: A) are very likely to give a stable protein having substantially the same binding to hNE, hCG, or some other serine protease as the parental sequence, B) are likely to give similar binding as the parent, or C) are likely to give a protein retaining the KuDom structure, but which are likely to affect the binding. Mutants in class C must be tested for affinity, which is relatively easy using a display-phage system, such as the one set forth in WO/02809. The affinity of hNE and hCG inhibitors is most sensitive to substitutions at positions 15, 16, 17, 18, 34, 39, 19, 13, 11, 20, 36 of BPTI, if the inhibitor is a mutant of ITI-D1, these positions must be converted to their ITI-D1 equivalents by aligning the cysteines in BPTI and ITI-D1.

Wild-type BPTI is not a good inhibitor of hNE. BPTI with a single K15L mutation exhibits a moderate affinity for HNE (K_{d}=2.9·10⁻⁹ M) (BECK88b). However, the amino terminal Kunitz domain (BI-8e) of the light chain of bovine inter-α-trypsin inhibitor has been generated by proteolysis and shown to be a potent inhibitor of HNE (K_{d}=4.4·10⁻¹¹ M) (ALBR83).

It has been proposed that the P1 residue is the primary determinant of the specificity and potency of BPTI-like molecules (SINH91, BECK88b, LASK80 and works cited therein). Although both BI-8e and BPTI(K15L) feature LEU at their respective P1 positions, there is a 66 fold difference in the affinities of these molecules for HNE. We therefore hypothesized that other structural features must contribute to the affinity of BPTI-like molecules for HNE.

A comparison of the structures of BI-8e and BPTI(K15L) reveals the presence of three positively charged residues at positions 39, 41, and 42 of BPTI which are absent in BI-8e. These hydrophilic and highly charged residues of BPTI are displayed on a loop which underlies the loop containing the P1 residue and is connected to it via a disulfide bridge. Residues within the underlying loop (in particular residue 39) participate in the interaction of BPTI with the surface of trypsin (BLOW72) and may contribute significantly to the tenacious binding of BPTI to trypsin. These hydrophilic residues might, however, hamper the docking of BPTI variants with HNE. Supporting this hypothesis, BI-8e displays a high affinity for HNE and contains no charged residues in residues 39-42. Hence, residues 39 through 42 of wild type BPTI were replaced with the corresponding residues (MGNG) of the human homologue of BI-8e. As we anticipated, a BPTI(K15L) derivative containing the MGNG 39-42 substitution exhibited a higher affinity for HNE than did the single substitution mutant BPTI(K15L). Mutants of BPTI with Met at position 39 are known, but positions 40-42 were not mutated simultaneously.

Tables 12 and 13 present the sequences of additional novel BPTI mutants with high affinity for hNE. We believe these mutants to have an affinity for hNE which is about an order of magnitude higher than that of BPTI (K15V, R17L). All of these mutants contain, besides the active site mutations shown in the Tables, the MGNG mutation at positions 39-42.

Although BPTI has been used in humans with very few adverse effects, a KuDom having much higher similarity to a human KuDom poses much less risk of causing an immune response. Thus, we transferred the active site changes found in EpiNE7 into the first KuDom of inter-α-trypsin inhibitor. For the purpose of this application, the numbering of the nucleic acid sequence for the ITI light chain gene is that of TRAB86 and that of the amino acid sequence is the one shown for UTI in FIG. 1 of GEBH86. The necessary coding sequence for ITI-DI is the 168 bases between positions 750 and 917 in the cDNA sequence presented in TRAB86. The amino acid sequence of human ITI-D1 is 56 amino acids long, extending from Lys-22 to Arg-77 of the complete ITI light chain sequence. The P1 site of ITI-DI is Met-36. Tables 21-22 present certain ITI mutants; note that the residues are numbered according to the homologus Kunitz domain of BPTI, i.e., with the P1 residue numbered 15. It should be noted that it is probably acceptable to truncate the amino-terminal of ITI-D1, at least up to the first residue homologous with BPTI.

The EpiNE7-inspired mutation (BPTI 15-19 region) of ITI-D1 significantly enhanced its affinity for hNE. We also discovered that mutation of a different part of the molecule (BPTI 1-4 region) provided a similar increase in affinity. When these two mutational patterns were combined, a synergistic increase in affinity was observed. Further mutations in nearby amino acids (BPTI 26, 31, 34) led to additional improvements in affinity.

The elastase-binding muteins of ITI-DI envisioned herein preferably differ from the wild-type domain at one or more of the following positions (numbered per BPTI): 1, 2, 4, 15, 16, 18, 19, 31 and 34. More preferably, they exhibit one or more of the following mutations: Lys1->Arg; Glu2->Pro; Ser4->Phe*; Met15->Val*, Ile; Gly16->Ala; THr18->Phe*; Ser19->Pro; Thr26->ALa; Glu31->Gln; Gln34->Val*. Introduction of one or more of the starred mutations is especially desirable, and, in one preferred embodiment, at least all of the starred mutations are present.

In a second series of embodiments, the present invention relates to Kunitz-type domains which inhibit HNE, but excludes those domains corresponding exactly to the lead domains of claims 1 and 3 of PCT/US92/01501. Preferably, such domains also differ from these lead domains by one or more mutations which are not class A substitutions, more preferably, not class A or B substitutions, and still more preferably, not class A, B or C substitutions, as defined in Table 9. Desirably, such domains are each more similar to one of the aforementioned reference proteins than to any of the lead proteins set forth in PCT/US92/01501.

The examples contain numerous examples of amino-acid sequences accompanied by DNA sequences that encode them. It is to be understood that the invention is not limited to the particular DNA sequence shown.

### EXAMPLE 1

### Expression and Display of BPTI, ITI-D1, and Other Kunitz Domains

Table 6 shows a display gene that encodes: 1) the M13 III signal peptide, 2) BPTI, and 3) the first few amino-acids of mature M13 III protein. Phage have been made in which this gene is the only iii-like gene so that all copies of III expressed are expected to be modified at the amino terminus of the mature protein. Substitutions in the BPTI domain can be made in the cassettes delimited by the AccIII, XhoI, PflMI, ApaI, BssHII, StuI, XcaI, EspI, SphI, or NarI (same recognition as KasI) sites. Table 10 gives amino-acid sequences of a number of Kunitz domains, some of which inhibit hNE. Each of the hNE-inhibiting sequences shown in Table 10 can be expressed as an intact hNE-binding protein or can be incorporated into a larger protein as a domain. Proteins that comprise a substantial part of one of the hNE-inhibiting sequences found in Table 10 are expected to exhibit hNE-inhibitory activity. This is particularly true if the sequence beginning with the first cysteine and continuing through the last cysteine is retained.

ITI domain 1 is a Kunitz domain as discussed below. The ability of display phage to be retained on matrices that display hNE is related to the affinity of the particular Kunitz domain (or other protein) displayed on the phage. Expression of the ITI domain 1::iii fusion gene and display of the fusion protein on the surface of phage were demonstrated by Western analysis and phage titer neutralization experiments. The infectivity of ITI-D1-display phage was blocked by up to 99% by antibodies that bind ITI while wild-type phage were unaffected.

Table 7 gives the sequence of a fusion gene comprising: a) the signal sequence of M13 III, b) ITI-D1, and c) the initial part of mature III of M13. The displayed ITI-D1 domain can be altered by standard methods including: i) oligonucleotide-directed mutagenesis of single-stranded phage DNA, and ii) cassette mutagenesis of RF DNA using the restriction sites (BglI, EagI, NcoI, StyI, PstI, and KasI (two sites)) designed into the gene.

### EXAMPLE 2

### Fractionation of MA-ITI-D1 Phage Bound to Agarose-Immobilized Protease Beads

To test if phage displaying the ITI-D1::III fusion protein interact strongly with the proteases human neutrophil elastase (hNE), aliquots of display phage were incubated with agarose-immobilized hNE beads (“hNE beads”). The beads were washed and bound phage eluted by pH fractionation as described in U.S. Pat. No. 5,223,409. The pHs used in the step gradient were 7.0, 6.0, 5.5, 5.0, 4.5, 4.0, 3.5, 3.0, 2.5, and 2.0. Following elution and neutralization, the various input, wash, and pH elution fractions were titered. Phage displaying ITI-D1 were compared to phage that display EpiNE-7.

The results of several fractionations are shown in Table 14 (EpiNE-7 or MA-ITI-D1 phage bound to hNE beads). The pH elution profiles obtained using the control display phage (EpiNE-7) were similar previous profiles (U.S. Pat. No. 5,223,409). About 0.3% of the EpiNE-7 display phage applied to the hNE beads eluted during the fractionation procedure and the elution profile had a maximum for elution at about pH 4.0.

The MA-ITI-D1 phage show no evidence of great affinity for hNE beads. The pH elution profiles for MA-ITI-D1 phage bound to hNE beads show essentially monotonic decreases in phage recovered with decreasing pH. Further, the total fractions of the phage applied to the beads that were recovered during the fractionation procedures were quite low: 0.002%.

Published values of Kᵢ for inhibition neutrophil elastase by the intact, large (Mᵣ=240,000) ITI protein range between 60 and 150 nM (SWAI88, ODOM90). Our own measurements of pH fraction of display phage bound to hNE beads show that phage displaying proteins with low affinity (>1 μM) for hNE are not bound by the beads while phage displaying proteins with greater affinity (nM) bind to the beads and are eluted at about pH 5. If the first Kunitz-type domain of the ITI light chain is entirely responsible for the inhibitory activity of ITI against hNE, and if this domain is correctly displayed on the MA-ITI-D1 phage, then it appears that the minimum affinity of an inhibitor for hNE that allows binding and fractionation of display phage on hNE beads is between 50 and 100 nM.

### EXAMPLE 3

### Alteration of the P1 Region of ITI-D1

We assume that ITI-D1 and EpiNE-7 have the same 3D configuration in solution as BPTI. Although EpiNE-7 and ITI-D1 are identical at positions 13, 17, 20, 32, and 39, they differ greatly in their affinities for hNE. To improve the affinity of ITI-D1 for hNE, the EpiNE-7 sequence Val₁₅-Ala₁₆-Met₁₇-Phe₁₈-Pro₁₉-Arg₂₀ SEQ ID NO:130 (bold, underscored amino acids are alterations) was incorporated into the ITI-D1 sequence by cassette mutagenesis between the EagI and StyI/NcoI sites shown in Table 7. Phage isolates containing the ITI-D1::III fusion gene with the EpiNE-7 changes around the P1 position are called MA-ITI-D1E7.

### EXAMPLE 4

### Fractionation of MA-ITI-D1E7 Phage

To test if ITI-D1E7-display phage bind hNE beads, pH elution profiles were measured. Aliquots of EpiNE-7, MA-ITI-D1, and MA-ITI-D1E7 display phage were incubated with hNE beads for three hours at room temperature (RT). The beads were washed and phage were eluted as described in U.S. Pat. No. 5,223,409, except that only three pH elutions were performed. These data are in Table 16. The pH elution profile of EpiNE-7 display phage is as described. MA-ITI-D1E7 phage show a broad elution maximum around pH 5. The total fraction of MA-ITI-D1E7 phage obtained on pH elution from hNE beads was about 40-fold less than that obtained using EpiNE-7 display phage.

The pH elution behavior of MA-ITI-D1E7 phage bound to hNE beads is qualitatively similar to that seen using BPTI[K15L]-III-MA phage. BPTI with the K15L mutation has an affinity for hNE of ≈3 nM. (Alterations and mutations are indicated by giving the original (wild-type) amino-acid type, then the position, and then the new amino-acid type; thus K15L means change Lys₁₅ to Leu.) Assuming all else remains the same, the pH elution profile for MA-ITI-D1E7 suggests that the affinity of the free ITI-D1E7 domain for hNE might be in the nM range. If this is the case, the substitution of the EpiNE-7 sequence in place of the ITI-D1 sequence around the P1 region has produced a 20- to 50-fold increase in affinity for hNE (assuming Kᵢ=60 to 150 nM for the unaltered ITI-D1).

If EpiNE-7 and ITI-D1E7 have the same solution structure, these proteins present the identical amino acid sequences to hNE over the interaction surface. Despite this similarity, EpiNE-7 exhibits a roughly 1000-fold greater affinity for hNE than does ITI-D1E7. This observation highlights the importance of non-contacting secondary residues in modulating interaction strengths.

Native ITI light chain is glycosylated at two positions, Ser₁₀ and Asn₄₅ (GEBH86). Removal of the glycosaminoglycan chains has been shown to decrease the affinity of the inhibitor for hNE about 5-fold (SELL87). Another potentially important difference between EpiNE-7 and ITI-D1E7 is that of net charge. The changes in BPTI that produce EpiNE-7 reduce the total charge on the molecule from +6 to +1. Sequence differences between EpiNE-7 and ITI-D1E7 further reduce the charge on the latter to −1. Furthermore, the change in net charge between these two molecules arises from sequence differences occurring in the central portions of the molecules. Position 26 is Lys in EpiNE-7 and is Thr in ITI-D1E7, while at position 31 these residues are Gln and Glu, respectively. These changes in sequence not only alter the net charge on the molecules but also position a negatively charged residue close to the interaction surface in ITI-D1E7. It may be that the occurrence of a negative charge at position 31 (which is not found in any other of the hNE inhibitors described here) destabilized the inhibitor-protease interaction.

### EXAMPLE 5

### Preparation of BITI-E7 Phage

Possible reasons for MA-ITI-D1E7 phage having lower affinity for hNE than do MA-EpiNE7 phage include: a) incorrect cleavage of the IIIsignal::ITI-D1E7::matureIII fusion protein, b) inappropriate negative charge on the ITI-D1E7 domain, c) conformational or dynamic changes in the Kunitz backbone caused by substitutions such as Phe₄ to Ser₄, and d) non-optimal amino acids in the ITI-D1E7:hNE interface, such as Q₃₄ or A₁₁.

To investigate the first three possibilities, we substituted the first four amino acids of EpiNE7 for the first four amino acids of ITI-D1E7. This substitution should provide a peptide that can be cleaved by signal peptidase-I in the same manner as is the IIIsignal::EpiNE7::matureIII fusion. Furthermore, Phe₄ of BPTI is part of the hydrophobic core of the protein; replacement with serine may alter the stability or dynamic character of ITI-D1E7 unfavorably. ITI-D1E7 has a negatively charged Glu at position 2 while EpiNE7 has Pro. We introduced the three changes at the amino terminus of the ITI-D1E7 protein (K1R, E2P, and S4F) by oligonucleotide-directed mutagenesis to produce BITI-E7; phage that display BITI-E7 are called MA-BITI-E7.

We compared the properties of the ITI-III fusion proteins displayed by phage MA-ITI-D1 and MA-BITI using Western analysis as described previously and found no significant differences in apparent size or relative abundance of the fusion proteins produced by either display phage strain. Thus, there are no large differences in the processed forms of either fusion protein displayed on the phage. By extension, there are also no large differences in the processed forms of the gene III fusion proteins displayed by MA-ITI-D1E7 and MA-EpiNE7. Large changes in protein conformation due to altered processing are therefore not likely to be responsible for the great differences in binding to hNE-beads shown by MA-ITI-D1E7 and MA-EpiNE7 display phage.

We characterized the binding properties to hNE-beads of MA-BITI and MA-BITI-E7 display phage using the extended pH fractionation procedure described in U.S. Pat. No. 5,223,409. The results are in Table 17. The pH elution profiles for MA-BITI and MA-BITI-E7 show significant differences from the profiles exhibited by MA-ITI-D1 and MA-ITI-D1E7. In both cases, the alterations at the putative amino terminus of the displayed fusion protein produce a several-fold increase in the fraction of the input display phage eluted from the hNE-beads.

The binding capacity of hNE-beads for display phage varies among preparations of beads and with age for each individual preparation of beads. Thus, it is difficult to directly compare absolute yields of phage from elutions performed at different times. For example, the fraction of MA-EpiNE7 display phage recovered from hNE-beads varies two-fold among the experiments shown in Tables 14, 16, and 17. However, the shapes of the pH elution profiles are similar. It is possible to correct somewhat for variations in binding capacity of hNE-beads by normalizing display phage yields to the total yield of MA-EpiNE7 phage recovered from the beads in a concurrent elution. When the data shown in Tables 14, 16, and 17 are so normalized, the recoveries of display phage, relative to recovered MA-EpiNE7, are shown in Table 3.

TABLE 3
Recovery of Display phage

| | Normalized fraction of |
|---|---|
| Display Phage strain | input |
| MA-ITI-D1 | 0.0067 |
| MA-BITI | 0.018 |
| MA-ITI-D1E7 | 0.027 |
| MA-BITI-E7 | 0.13 |

Thus, the changes in the amino terminal sequence of the displayed protein produce a three- to five-fold increase in the fraction of display phage eluted from hNE-beads.

In addition to increased binding, the changes introduced into MA-BITI-E7 produce phage that elute from hNE-beads at a lower pH than do the parental MA-ITI-D1E7 phage. While the parental display phage elute with a broad pH maximum centered around pH 5.0, the pH elution profile for MA-BITI-E7 display phage has a pH maximum at around pH 4.75 to pH 4.5.

The pH elution maximum of the MA-BITI-E7 display phage is between the maxima exhibited by the BPTI(K15L) and BPTI(K15V, R17L) display phage (pH 4.75 and pH 4.5 to pH 4.0, respectively) described in U.S. Pat. No. 5,223,409. From the pH maximum exhibited by the display phage we predict that the BITI-E7 protein free in solution may have an affinity for hNE in the 100 pM range. This would represent an approximately ten-fold increase in affinity for hNE over that estimated above for ITI-D1E7.

As was described above, Western analysis of phage proteins show that there are no large changes in gene III fusion proteins upon alteration of the amino terminal sequence. Thus, it is unlikely that the changes in affinity of display phage for hNE-beads can be attributed to large-scale alterations in protein folding resulting from altered (“correct”) processing of the fusion protein in the amino terminal mutants. The improvements in binding may in part be due to: 1) the decrease in the net negative charge (−1 to 0) on the protein arising from the Glu to Pro change at position 2, or 2) increased protein stability resulting from the Ser to Phe substitution at residue 4 in the hydrophobic core of the protein, or 3) the combined effects of both substitutions.

### EXAMPLE 6

### Production and Properties of MA-BITI-E7-1222 and MA-BITI-E7-141

Within the presumed Kunitz:hNE interface, BITI-E7 and EpiNE7 differ at only two positions: 11 and 34. In EpiNE7 these residues are Thr and Val, respectively. In BITI-E7 they are Ala and Gln. In addition BITI-E7 has Glu at 31 while EpiNE7 has Gln. This negative charge may influence binding although the residue is not directly in the interface. We used oligonucleotide-directed mutagenesis to investigate the effects of substitutions at positions 11, 31 and 34 on the protease:inhibitor interaction.

ITI-D1 derivative BITI-E7-1222 is BITI-E7 with the alteration A11T. ITI-D1 derivative BITI-E7-141 is BITI-E7 with the alterations E31Q and Q34V; phage that display the presence of these proteins are MA-BITI-E7-1222 and MA-BITI-E7-141. We determined the binding properties to hNE-beads of MA-BITI-E7-1222 and MA-BITI-E7-141 display phage using the extended pH fractionation protocol described previously. The results are in Tables 18 (for MA-BITI-E7 and MA-BITI-E7-1222) and 19 (for MA-EpiNE7 and MA-BITI-E7-141). The pH elution profiles for the MA-BITI-E7 and MA-BITI-E7-1222 phage are almost identical. Both phage strains exhibit pH elution profiles with identical maxima (between pH 5.0 and pH 4.5) as well as the same total fraction of input phage eluted from the hNE-beads (0.03%). Thus, the T11A substitution in the displayed ITI-D1 derivative has no appreciable effect on the binding to hNE-beads.

In contrast, the changes at positions 31 and 34 strongly affect the hNE-binding properties of the display phage. The elution profile pH maximum of MA-BITI-E7-141 phage is shifted to lower pH relative to the parental MA-BITI-E7 phage. Further, the position of the maximum (between pH 4.5 and pH 4.0) is identical to that exhibited by MA-EpiNE7 phage in this experiment. Finally, the MA-BITI-E7-141 phage show a ten-fold increase, relative to the parental MA-BITI-E7, in the total fraction of input phage eluted from hNE-beads (0.3% vs 0.03%). The total fraction of MA-BITI-E7-141 phage eluted from the hNE-beads is nearly twice that of MA-EpiNE7 phage.

The above results show that binding by MA-BITI-E7-141 display phage to hNE-beads is comparable to that of MA-EpiNE7 phage. If the two proteins (EpiNE7 and BITI-E7-141) in solution have similar affinities for hNE, then the affinity of the BITI-E7-141 protein for hNE is on the order of 1 pM. Such an affinity is approximately 100-fold greater than that estimated above for the parental protein (BITI-E7) and is 10⁵ to 10⁶ times as great as the affinity for hNE reported for the intact ITI protein.

### EXAMPLE 7

### Mutagenesis of BITI-E7-141

BITI-E7-141 differs from ITI-D1 at nine positions (1, 2, 4, 15, 16, 18, 19, 31, and 34). To obtain the protein having the fewest changes from ITI-D1 while retaining high specific affinity for hNE, we have investigated the effects of reversing the changes at positions 1, 2, 4, 16, 19, 31, and 34. The derivatives of BITI-E7-141 that were tested are MUT1619, MUTP1, and MUTT26A. The derivatives of BITI that were tested are AMINO1 and AMINO2. The derivative of BITI-E7 that was tested is MUTQE. All of these sequences are shown in Table 10. MUT1619 restores the ITI-D1 residues Ala₁₆ and Ser₁₉. The sequence designated “MUTP1” asserts the amino acids I₁₅, G₁₆, S₁₉ in the context of BITI-E7-141. It is likely that M₁₇ and F₁₈ are optimal for high affinity hNE binding. G₁₆ and S₁₉ occurred frequently in the high affinity hNE-binding BPTI-variants obtained from fractionation of a library of BPTI-variants against hNE (ROBE92). Three changes at the putative amino terminus of the displayed ITI-D1 domain were introduced to produce the MA-BITI series of phage. AMINO1 carries the sequence K₁-E₂ while AMINO2 carries K₁-S₄. Other amino acids in the amino-terminal region of these sequences are as in ITI-D1. MUTQE is derived from BITI-E7-141 by the alteration Q31E (reasseting the ITI-D1 w.t. residue). Finally, the mutagenic oligonucleotide MUTT26A is intended to remove a potential site of N-linked glycosylation, N₂₄-G₂₅-T₂₆. In the intact ITI molecule isolated from human serum, the light chain polypeptide is glycosylated at this site (N₄₅, ODOM90). It is likely that N₂₄ will be glycosylated if the BITI-E7-141 protein is produced via eukaryotic expression. Such glycosylation may render the protein immunogenic when used for long-term treatment. The MUTT26A contains the alteration T26A and removes the potential glycosylation site with minimal changes in the overall chemical properties of the residue at that position. In addition, an Ala residue is frequently found in other BPTI homologues at position 26 (see Table 34 of U.S. Pat. No. 5,223,409). Mutagenesis was performed on ssDNA of MA-BITI-E7-141 phage.

### EXAMPLE 8

### hNE-Binding Properties of Mutagenized MA-BITI-E7-141 Display Phage

Table 20 shows pH elution data for various display phage eluted from hNE-beads. Total pfu applied to the beads are in column two. The fractions of this input pfu recovered in each pH fraction of the abbreviated pH elution protocol (pH 7.0, pH 3.5, and pH 2.0) are in the next three columns. For data obtained using the extended pH elution protocol, the pH 3.5 listing represents the sum of the fractions of input recovered in the pH 6.0, pH 5.5, pH 5.0, pH 4.5, pH 4.0, and pH 3.5 elution samples. The pH 2.0 listing is the sum of the fractions of input obtained from the pH 3.0, pH 2.5, and pH 2.0 elution samples. The total fraction of input pfu obtained throughout the pH elution protocol is in the sixth column. The final column of the table lists the total fraction of input pfu recovered, normalized to the value obtained for MA-BITI-E7-141 phage.

Two factors must be considered when making comparisons among the data shown in Table 20. The first is that due to the kinetic nature of phage release from hNE-beads and the longer time involved in the extended pH elution protocol, the fraction of input pfu recovered in the pH 3.5 fraction will be enriched at the expense of the pH 2.0 fraction in the extended protocol relative to those values obtained in the abbreviated protocol. The magnitude of this effect can be seen by comparing the results obtained when MA-BITI-E7-141 display phage were eluted from hNE-beads using the two protocols. The second factor is that, for the range of input pfu listed in Table 20, the input pfu influences recovery. The greater the input pfu, the greater the total fraction of the input recovered in the elution. This effect is apparent when input pfu differ by more than a factor of about 3 to 4. The effect can lead to an overestimate of affinity of display phage for hNE-beads when data from phage applied at higher titers is compared with that from phage applied at lower titers.

With these caveats in mind, we can interpret the data in Table 20. The effects of the mutations introduced into MA-BITI-E7-141 display phage (“parental”) on binding of display phage to hNE-beads can be grouped into three categories: those changes that have little or no measurable effects, those that have moderate (2- to 3-fold) effects, and those that have large (>5-fold) effects.

The MUTT26A and MUTQE changes appear to have little effect on the binding of display phage to hNE-beads. In terms of total pfu recovered, the display phage containing these alterations bind as well as the parental to hNE-beads. Indeed, the pH elution profiles obtained for the parental and the MUTT26A display phage from the extended pH elution protocol are indistinguishable. The binding of the MUTTQE display phage appears to be slightly reduced relative to the parental and, in light of the applied pfu, it is likely that this binding is somewhat overestimated.

The sequence alterations introduced via the MUTP1 and MUT1619 oligonucleotides appear to reduce display phage binding to hNE-beads about 2- to 3-fold. In light of the input titers and the distributions of pfu recovered among the various elution fractions, it is likely that 1) both of these display phage have lower affinities for hNE-beads than do MA-EpiNE7 display phage, and 2) the MUT1619 display phage have a greater affinity for hNE-beads than do the MUTP1 display phage.

The sequence alterations at the amino terminus of BITI-E7-14 appear to reduce binding by the display phage to hNE-beads at least ten fold. The AMINO2 changes are likely to reduce display phage binding to a substantially greater extent than do the AMINO1 changes.

On the basis of the above interpretations of the data in Table 20, we can conclude that:
1.) The substitution of ALA for THR at position 26 in ITI-D1 and its derivatives has no effect on the interaction of the inhibitor with hNE. Thus, the possibility of glycosylation at Asn₂₄ of an inhibitor protein produced in eukaryotic cell culture can be avoided with no reduction in affinity for hNE.
2.) The increase in affinity of display phage for hNE-beads from the changes E31Q and Q34V results primarily from the Val substitution at 34.
3.) All three changes at the amino terminal region of ITI-D1 (positions 1, 2, and 4) influence display phage binding to hNE-beads to varying extents. The S4F alteration seems to have about the same effect as does E2P. The change at position 1 appears to have only a small effect.
4.) The changes in the region around the P1 residue in BITI-E7-141 (position 15) influence display phage binding to hNE. The changes A16G and P19S appear to reduce the affinity of the inhibitor somewhat (perhaps 3-fold). The substitution of I15V further reduces binding.

BITI-E7-141 differs from ITI-D1 at nine positions. From the discussion above, it appears likely that a high affinity hNE-inhibitor based on ITI-D1 could be constructed that would differ from the ITI-D1 sequence at only five or six positions. These differences would be: Pro at position 2, Phe at position 4, Val at position 15, Phe at position 18, Val at position 34, and Ala at position 26. If glycosylation of Asn₂₄ is not a concern Thr could be retained at 26.

Summary: Estimated Affinities of Isolated ITI-D1 Derivatives for hNE

On the basis of display phage binding to and elution from hNE beads, it is possible to estimate affinities for hNE that various derivatives of ITI-D1 may display free in solution. These estimates are summarized in Table 8.

hNE Inhibitors Derived from ITI Domain 2

In addition to hNE inhibitors derived from ITI-D1, the present invention comprises hNE inhibitors derived from ITI-D2. These inhibitors have been produced in Pichia pastoris in good yield. EPI-HNE-4 inhibits human neutrophil elastase with a K_{D}≈5 pM.

Purification and Properties of EPI-HNE Proteins

I. EPI-HNE Proteins.

### EXAMPLE 9

### Amino-Acid Sequences of EPI-HNE-3 and EPI-HNE-4

Table 10 gives amino acid sequences of four human-neutrophil-elastase (hNE) inhibitor proteins: EPI-HNE-1 (which is identical to EpiNE1), EPI-HNE-2, EPI-HNE-3, and EPI-HNE-4. These proteins have been derived from the parental Kunitz-type domains shown. Each of the proteins is shown aligned to the parental domain using the six cysteine residues (shaded) characteristic of the Kunitz-type domain. Residues within the inhibitor proteins that differ from those in the parental protein are in upper case. Entire proteins having the sequences EPI-HNE-1, EPI-HNE-2, EPI-HNE-3, and EPI-HNE-4 (Table 10) have been produced. Larger proteins that comprise one of the hNE-inhibiting sequences are expected to have potent hNE-inhibitory activity; EPI-HNE-1, EPI-HNE-2, EPI-HNE-3, and EPI-HNE-4 are particularly preferred. It is expected that proteins that comprise a significant part of one of the hNE-inhibiting sequences found in Table 10 (particularly if the sequence starting at or before the first cysteine and continuing through or beyond the last cysteine is retained) will exhibit potent hNE-inhibitory activity.

The hNE-inhibitors EPI-HNE-1 and EPI-HNE-2 are derived from the bovine protein BPTI (aprotinin). Within the Kunitz-type domain, these two inhibitors differ from BPTI at the same eight positions: K15I, R17F, I18F, I19P, R39M, A40G, K41N, and R42G. In addition, EPI-HNE-2 differs from both BPTI and EPI-HNE-1 in the presence of four additional residues (EAEA) present at the amino terminus. These residues were added to facilitate secretion of the protein in Pichia pastoris.

EPI-HNE-3 is derived from the second Kunitz domain of the light chain of the human inter-α-trypsin inhibitor protein (ITI-D2). The amino acid sequence of EPI-HNE-3 differs from that of ITI-D2(3-58) at only four positions: R15I, I18F, Q19P and L20R. EPI-HNE-4 differs from EPI-HNE-3 by the substitution A3E (the amino-terminal residue) which both facilitates secretion of the protein in P. pastoris and improves the K_{D} for hNE. Table 30 gives some physical properties of the hNE inhibitor proteins. All four proteins are small, high-affinity (Kᵢ=2 to 6 pM), fast-acting (kₒₙ=4 to 11×10⁶ M⁻¹s⁻¹) inhibitors of hNE.

II. Production of the hNE-Inhibitors EPI-HNE-2, EPI-HNE-3, and EPI-HNE-4.

### EXAMPLE 10

### Pichia pastoris Production System

Transformed strains of Pichia pastoris were used to express the various EPI-HNE proteins derived from BPTI and ITI-D2. Protein expression cassettes are cloned into the plasmid pHIL-D2 using the BstBI and EcoRI sites (Table 11). The DNA sequence of pHIL-D2 is given in Table 23. The cloned gene is under transcriptional control of P. pastoris upstream (labeled “aox1 5′”) aox1 gene promoter and regulatory sequences (dark shaded region) and downstream polyadenylation and transcription termination sequences (second cross-hatched region, labeled “aox1 3′”). P. pastoris GS115 is a mutant strain containing a non-functional histidinol dehydrogenase (his4) gene. The his4 gene contained on plasmid pHIL-D2 and its derivatives can be used to complement the histidine deficiency in the host strain. Linearization of plasmid pHIL-D2 at the indicated SacI site directs plasmid incorporation into the host genome at the aox1 locus by homologous recombination during transformation. Strains of P. pastoris containing integrated copies of the expression plasmid will express protein genes under control of the aox1 promoter when the promoter is activated by growth in the presence of methanol as the sole carbon source.

We have used this high density Pichia pastoris production system to produce proteins by secretion into the cell culture medium. Expression plasmids were constructed by ligating synthetic DNA sequences encoding the S. cerevisiae mating factor α prepro peptide fused directly to the amino terminus of the desired hNE inhibitor into the plasmid pHIL-D2 using the BstBI and the EcoRI sites shown. Table 24 gives the DNA sequence of a BstBI-to-EcoRI insert that converts pHIL-D2 into pHIL-D2(MFα-PrePro::EPI-HNE-3). In this construction, the fusion protein is placed under control of the upstream inducible P. pastoris aox1 gene promoter and the downstream aox1 gene transcription termination and polyadenylation sequences. Expression plasmids were linearized by SacI digestion and the linear DNA was incorporated by homologous recombination into the genome of the P. pastoris strain GS115 by spheroplast transformation. Regenerated spheroplasts were selected for growth in the absence of added histidine, replated, and individual isolates were screened for methanol utilization phenotype (mut⁺), secretion levels, and gene dose (estimated via Southern hybridization experiments). High level secretion stains were selected for production of hNE inhibitors: PEY-33 for production of EPI-HNE-2 and PEY-43 for production of EPI-HNE-3. In both of these strains, we estimate that four copies of the expression plasmid are integrated as a tandem array into the aox1 gene locus.

To facilitate alteration of the Kunitz-domain encoding segment of pHIL-D2 derived plasmids, we removed two restriction sites given in Table 11: the BstBI at 4780 and the AatII site at 5498. Thus, the Kunitz-domain encoding segment is bounded by unique AatII and EcoRI sites. The new plasmids are called pD2pick(“insert”) where “insert” defines the domain encoded under control of the aox1 promoter. Table 26 gives the DNA sequence of pD2pick(MFα::EPI-HNE-3). Table 27 gives a list of restriction sites in pD2pick(MFα::EPI-HNE-3).

EPI-HNE-4 is encoded by pD2pick(MFαPrePro::EPI-HNE-4) which differs from pHIL-D2 in that: 1) the AatII/EcoRI segment of the sequence given in Table 24 is replaced by the segment shown in Table 25 and 2) the changes in the restriction sites discussed above have been made. Strain PEY-53 is P. pastoris GS115 transformed with pD2pick(MFα::EPI-HNE-4).

### EXAMPLE 11

### Protein Production

To produce the proteins, P. pastors strains were grown in mixed-feed fermentations similar to the procedure described by Digan et al. (DIGA89). Although others have reported production of Kunitz domains in P. pastoris, it is well known that many secretion systems involve proteases. Thus, it is not automatic that an altered Kunitz domain having a high potency in inhibiting hNE could be secreted from P. pastoris because the new inhibitor might inhibit some key enzyme in the secretion pathway. Nevertheless, we have found that P. pastoris can secrete hNE inhibitors in high yield.

Briefly, cultures were first grown in batch mode with glycerol as the carbon source. Following exhaustion of glycerol, the culture was grown for about four hours in glycerol-limited feed mode to further increase cell mass and to derepress the aox1 promoter. In the final production phase, the culture was grown in methanol-limited feed mode. During this phase, the aox1 promoter is fully active and protein is secreted into the CM.

Table 34 and Table 35 give the kinetics of cell growth (estimated as A600) and protein secretion (mg/l) for cultures of PEY-33 and PEY-43 during the methanol-limited feed portions of the relevant fermentations. Concentrations of the inhibitor proteins in the fermentation cultures were determined from in vitro assays of hNE inhibition by diluted aliquots of cell-free culture media obtained at the times indicated. Despite similarities in gene dose, fermentation conditions, cell densities, and secretion kinetics, the final concentrations of inhibitor proteins secreted by the two strains differ by nearly an order of magnitude. The final concentration of EPI-HNE-2 in the PEY-33 fermentation CM was 720 mg/l. The final concentration of EPI-HNE-3 in the PEY-43 fermentation CM was 85 mg/l. The differences in final secreted protein concentrations may result from idiosyncratic differences in the efficiencies with which the yeast synthesis and processing systems interact with the different protein sequences.

Strain PEY-33 secreted EPI-HNE-2 protein into the CM as a single molecular species which amino acid composition and N-terminal sequencing reveled to be the correctly-processed Kunitz domain with the sequence shown in Table 29. The major molecular species produced by PEY-43 cultures was the properly-processed EPI-HNE-3 protein. However, this strain also secreted a small amount (about 15% to 20% of the total EPI-HNE-3) of incorrectly-processed material. This material proved to be a mixture of proteins with amino terminal extensions (primarily nine or seven residues in length) arising from incorrect cleavage of the MF α PrePro leader peptide from the mature Kunitz domain. The correctly processed protein was purified substantially free of these contaminants as described below.

III. Purification of the hNE-Inhibitors EPI-HNE-2 and EPI-HNE-3.

The proteins can be readily purified from fermenter CM by standard biochemical techniques. The specific purification procedure varies with the specific properties of each protein as described below.

### EXAMPLE 12

### Purification of EPI-HNE-2

Table 31 gives particulars of the purification of EPI-HNE-2, lot 1. The PEY-33 fermenter culture was harvested by centrifugation at 8000×g for 15 min and the cell pellet was discarded. The 3.3 liter supernatant fraction was microfiltered used a Minitan Ultrafiltration System (Millipore Corporation, Bedford, Mass.) equipped with four 0.2μ filter packets.

The filtrate obtained from the microfiltration step was used in two subsequent ultrafiltration steps. First, two 30K ultrafiltrations were performed on the 0.2μ microfiltrate using the Minitan apparatus equipped with eight 30,000 NMWL polysulfone filter plates (#PLTK0MP04, Millipore Corporation, Bedford, Mass.). The retentate solution from the first 30K ultrafiltration was diluted with 10 mM NaCitrate, pH=3.5, and subjected to a second 30K ultrafiltration. The two 30K ultrafiltrates were combined to give a final volume of 5 liters containing about 1.4 gram of EPI-HNE-2 protein (estimated from hNE-inhibition measurements).

The 30K ultrafiltrate was concentrated with change of buffer in the second ultrafiltration step using the Minitan apparatus equipped with eight 5,000 NMWL filter plates (#PLCC0MP04, Millipore Corporation, Bedford, Mass.). At two times during the 5K ultrafiltration, the retentate solution was diluted from about 300 ml to 1.5 liters with 10 mM NaCitrate, pH=3.5. The final 5K ultrafiltration retentate (Ca. 200 ml) was diluted to a final volume of 1000 ml with 10 mM NaCitrate, pH-3.5.

EPI-HNE-2 protein was obtained from the 5K ultrafiltration retentate solution by ammonium sulfate precipitation at RT. 100 ml of 0.25 M ammonium acetate, pH=3.2, (1/10 volume) was added to the 5K ultrafiltration retentate, followed by one final volume (1.1 liter) of 3 M ammonium sulfate. Following a 30 minute incubation at RT, precipitated material was pelleted by centrifugation at 10,000×g for 45 minutes. The supernatant solution was removed, the pellet was dissolved in water in a final volume of 400 ml, and the ammonium sulfate precipitation was repeated using the ratios described above. The pellet from the second ammonium sulfate precipitation was dissolved in 50 mM sodium acetate, pH=3.5 at a final volume of 300 ml.

Residual ammonium sulfate was removed from the EPI-HNE-2 preparation by ion exchange chromatography. The solution from the ammonium sulfate precipitation step was applied to a strong cation-exchange column (50 ml bed volume Macroprep 50S) (Bio-Rad Laboratories, Inc, Hercules, Calif.) previously equilibrated with 10 mM NaCitrate, pH=3.5. After loading, the column was washed with 300 ml of 10 mM NaCitrate, pH=3.5. EPI-HNE-2 was then batch-eluted from the column with 300 ml of 50 mM NH₄OAc, pH=6.2. Fractions containing EPI-HNE-2 activity were pooled and the resulting solution was lyophilized. The dried protein powder was dissolved in 50 ml dH₂O and the solution was passed through a 0.2μ filter (#4192, Gelman Sciences, Ann Arbor, Mich.). The concentration of the active inhibitor in the final stock solution was determined to be 2 mM (13.5 mg/ml). This stock solution (EPI-HNE-2, Lot 1) has been stored as 1 ml aliquots at 4° C. and −70° C. for more than 11 months with no loss in activity.

Table 31 summarizes the yields and relative purity of EPI-HNE-2 at various steps in the purification procedure. The overall yield of the purification procedure was about 30%. The major source of loss was retention of material in the retentate fractions of the 0.2μ microfiltration and 30k ultrafiltration steps.

### EXAMPLE 13

### Purification of EPI-HNE-3

Purification of EPI-HNE-3, lot 1, is set out in Table 32. The PEY-43 fermenter culture was harvested by centrifugation at 8,000×g for 15 min and the cell pellet was discarded. The supernatant solution (3100 ml) was microfiltered through 0.2μ Minitan packets (4 packets). After the concentration, a diafiltration of the retentate was performed so that the final filtrate volume from the 0.2μ filtration was 3300 ml.

A 30K ultrafiltration was performed on the filtrate from the 0.2μ microfiltration step. When the retentate volume had been reduced to 250 ml, a diafiltration of the retentate was performed at a constant retentate volume (250 ml) for 30 min at a rate of 10 ml/min. The resulting final volume of filtrate was 3260 ml.

EPI-HNE-3 protein and other medium components were found to precipitate from solution when the fermenter CM was concentrated. For this reason, the 5k ultrafiltration step was not performed.

Properly processed EPI-HNE-3 was purified substantially free of mis-processed forms and other fermenter culture components by ion exchange chromatography. A 30 ml bed volume strong cation ion exchange column (Macroprep 50S) was equilibrated with 10 mM NaCitrate pH=3.5. The 30K ultrafiltration filtrate was applied to the column and binding of EPI-HNE-3 to the column was confirmed by demonstrating the complete loss of inhibitor activity in the column flow through. The column was then washed with 300 ml of 10 mM NaCitrate, pH=3.5.

To remove EPI-HNE-3 from the column, we sequentially eluted it with 300 ml volumes of the following solutions:
100 mM ammonium acetate, pH=3.5
50 mM ammonium acetate, pH=4.8
50 mM ammonium acetate, pH=6.0
50 mM ammonium acetate, pH=6.0, 0.1 M NaCl
50 mM ammonium acetate, pH=6.0, 0.2 M NaCl
50 mM ammonium acetate, pH=6.0, 0.3 M NaCl
50 mM ammonium acetate, pH=6.0, 0.4 M NaCl
50 mN Tris/Cl pH=8.0, 1.0 NaCl
   The majority of the EPI-HNE-3 eluted in two 50 mM ammonium acetate, pH=6.0 fractions. The 0.1 M NaCl fraction contained about 19% of the input EPI-HNE-3 activity (28 mg of 159 mg input) and essentially all of the mis-processed forms of EPI-HNE-3. The 0.2M NaCl fraction contained about 72% (114 mg) of the input EPI-HNE-3 and was almost completely free of the higher molecular weight mis-processed forms and other UV-absorbing contaminants. The fractions from the 50 mM ammonium acetate, pH=6.0, 0.2 M NaCl elution having the highest concentrations of EPI-HNE-3 were combined (95 mg).

An ammonium sulfate precipitation was performed on the 0.2 M NaCl, pH=6.0 ion exchange column eluate. 800 ml of 3 M ammonium sulfate was added to 160 ml of eluate solution (final ammonium sulfate concentration=2.5 M) and the mixture was incubated at RT for 18 hours. The precipitated material was then pelleted by centrifugation at 10,000×g for 45 minutes. The supernatant fluid was discarded and the pelleted material was dissolved in 100 ml of water.

Residual ammonium sulfate was removed from the EPI-HNE-3 preparation by batch ion exchange chromatography. The pH of the protein solution was adjusted to 3.0 with diluted (1/10) HOAc and the solution was then applied to a 10 ml bed volume Macroprep 50S column that had been equilibrated with 10 mM NaCitrate, pH=3.5. Following sample loading, the column was washed with 100 ml of 10 mM NaCitrate, pH=3.5 followed by 100 ml of dH₂O. EPI-HNE-3 was eluted from the column with 100 ml of 50 mM NH₄CO₃, pH=9.0. pH9 fractions having the highest concentrations of EPI-HNE-3 were combined (60 mg) and stored at 4° C. for 7 days before lyophilization.

The lyophilized protein powder was dissolved in 26 ml dH₂O and the solution was passed through a 0.2μ filter (#4912, Gelman Sciences, Ann Arbor, Mich.). The concentration of active inhibitor in the final stock solution was found to be 250 μM (1.5 mg/ml). This stock solution (EPI-HNE-3, Lot 1) has been stored as 1 ml aliquots at −70° C. for more than six months with no loss of activity. EPI-HNE-3 stored in water solution (without any buffering) deteriorated when kept at 4° C. After five months, about 70% of the material was active with a Kᵢ of about 12 pM.

Table 32 gives the yield and relative purity of EPI-HNE-3 at various steps in the purification procedure. A major purification step occurred at the first ion exchange chromatography procedure. The ammonium sulfate precipitation step provided only a small degree of further purification. Some loss of inhibitor activity occurred on incubation at pH=9 (See pH stability data). The production and purification of EPI-HNE-1 and EPI-HNE-4 were analogous to that of EPI-HNE-2 and EPI-HNE-3.

### EXAMPLE 14

### Tricine-PAGE Analysis of EPI-HNE-2 and EPI-HNE-3

The high resolution tricine gel system of Schagger and von Jagow (SCHA87) was used to analyze the purified proteins produced (vide supra). For good resolution of the low molecular weight EPI-HNE proteins we used a 16.5% resolving layer with 10% separating and 4% stacking layers. Following silver staining, we inspected a gel having:
Lane 1: EPI-HNE-2 25 ng,
Lane 2: EPI-HNE-2 50 ng,
Lane 3: EPI-HNE-2 100 ng,
Lane 4: EPI-HNE-2 200 ng,
Lane 5: EPI-HNE-3 25 ng,
Lane 6: EPI-HNE-3 50 ng,
Lane 7: EPI-HNE-3 100 ng,
Lane 8: EPI-HNE-3 200 ng, and
Lane 9: Molecular-weight standards: RPN 755, (Amersham Corporation, Arlington Heights, Ill.).
   Stained proteins visible on the gel and their molecular weights in Daltons are: ovalbumin (46,000), carbonic anhydrase (30,000), trypsin inhibitor (21,500), lysozyme (14,300), and aprotinin (6,500). The amount of protein loaded was determined from measurements of hNE-inhibition. We found the following features. EPI-HNE-2, Lot 1 shows a single staining band of the anticipated size (ca. 6,700 D) at all loadings. Similarly, EPI-HNE-3, Lot 1 protein shows a single staining band of the anticipated size (ca. 6,200 D). At the highest loading, traces of the higher molecular weight (ca. 7,100 D) incorrectly processed form can be detected. On the basis of silver-stained high-resolution PAGE analysis, the purity of both protein preparations is assessed to be significantly greater than 95%.
   IV. Properties of EPI-HNE-2 and EPI-HNE-3.
   A. Inhibition of hNE.

### EXAMPLE 15

### Measured K_{D}s of EPI-HNE Proteins for hNE

Inhibition constants for the proteins reacting with hNE (Kᵢ) were determined using RT measurements of hydrolysis of a fluorogenic substrate (N-methoxysuccinyl-Ala-Ala-Pro-Val-7-amino-4-methylcoumarin, Sigma M-9771) by hNE. For these measurements, aliquots of the appropriately diluted inhibitor stocks were added to 2 ml solutions of 0.847 nM hNE in reaction buffer (50 mM Tris-Cl, pH=8.0, 150 mM NaCl, 1 mM CaCl₂, 0.25% Triton-X-100) in plastic fluorescence cuvettes. The reactions were incubated at RT for 30 minutes. At the end of the incubation period, the fluorogenic substrate was added at a concentration of 25 μM and the time course for increase in fluorescence at 470 nm (excitation at 380 nm) due to enzymatic substrate cleavage was recorded using a spectrofluorimeter (Perkin-Elmer 650-15) and strip chart recorder. We did not correct for competition between substrate and inhibitor because (S₀/Kₘ) is 0.07 (where S₀ is the substrate concentration and Kₘ is the binding constant of the substrate for hNE). Kᵢ is related to Kₐₚₚₐᵣₑₙₜ by Kᵢ=Kₐₚₚₐᵣₑₙₜ×(1/(1+(S₀/Kₘ))). The correction is small compared to the possible errors in Kₐₚₚₐᵣₑₙₜ. Rates of enzymatic substrate cleavage were determined from the linear slopes of the recorded increases in fluorescence. The percent residual activity of hNE in the presence of the inhibitor was calculated as the percentage of the rate of fluorescence increase observed in the presence of the inhibitor to that observed when no added inhibitor was present.

We recorded data used to determine Kᵢ for EPI-HNE-2 and EPI-HNE-3 reacting with hNE. Data obtained as described above are recorded as percent residual activity plotted as a function of added inhibitor. Values for Kᵢ and for active inhibitor concentration in the stock are obtained from a least-squares fit program. From the data, Kᵢ values for EPI-HNE-2 and for EPI-HNE-3 reacting with hNE at RT were calculated to be 4.8 pM and 6.2 pM, respectively. Determinations of Kᵢ for EPI-HNE-2 and EPI-HNE-3 reacting with hNE are given in Table 36 and Table 37.

The kinetic on-rates for the inhibitors reacting with hNE (kₒₙ) were determined from measurements of progressive inhibition of substrate hydrolytic activity by hNE following addition of inhibitor. For these experiments, a known concentration of inhibitor was added to a solution of hNE (0.847 nM) and substrate (25 μM) in 2 ml of reaction buffer in a plastic fluorescence cuvette. The change in fluorescence was recorded continuously following addition of the inhibitor. In these experiments, sample fluorescence did not increase linearly with time. Instead, the rate of fluorescence steadily decreased reflecting increasing inhibition of hNE by the added inhibitor. The enzymatic rate at selected times following addition of the inhibitor was determined from the slope of the tangent to the fluorescence time course at that time.

The kinetic constant kₒₙ for EPI-HNE-2 reacting with hNE was determined as follows. EPI-HNE-2 at 1.3 nM was added to buffer containing 0.867 nM hNE (I:E=1.5:1) at time 0. Measured percent residual activity was recorded as a function of time after addition of inhibitor. A least-squares fit program was used to obtain the value of kₒₙ=4.0×10⁶ M⁻¹s⁻¹.

The kinetic off rate, k_{off}, is calculated from the measured values of Kᵢ and kₒₙ as:
k_{off}=K_{D}−kₒₙ
The values from such measurements are included in Table 30. The EPI-HNE proteins are small, high affinity, fast acting inhibitors of hNE.
B. Specificity.

### EXAMPLE 16

### Specificity of EPI-HNE Proteins

We attempted to determine inhibition constants for EPI-HNE proteins reacting with several serine proteases. The results are summarized in Table 33. In all cases except chymotrypsin, we were unable to observe any inhibition even when 10 to 100 μM inhibitor was added to enzyme at concentrations in the nM range. In Table 33, our calculated values for Kᵢ (for the enzymes other than chymotrypsin) are based on the conservative assumption of less than 10% inhibition at the highest concentrations of inhibitor tested. For chymotrypsin, the Kᵢ is about 10 μM and is probably not specific.

C. In Vitro Stability.

### EXAMPLE 17

### Resistance to Oxidative Inactivation

Table 39 shows measurements of the susceptibility of EPI-HNE proteins to oxidative inactivation as compared with that of two other natural protein hNE inhibitors: α 1 Protease Inhibitor (API) and Secretory Leucocyte Protease Inhibitor (SLPI). API (10 μM), SLPI (8.5 μM), EPI-HNE-1 (5 μM), EPI-HNE-2 (10 μM), EPI-HNE-3 (10 μM), and EPI-HNE-4 (10 μM) were exposed to the potent oxidizing agent, Chloramine-T, at the indicated oxidant:inhibitor ratios in 50 mM phosphate buffer, pH=7.0 for 20 minutes at RT. At the end of the incubation period, the oxidation reactions were quenched by adding methionine to a final concentration of 4 mM. After a further 10 minute incubation, the quenched reactions were diluted and assayed for residual inhibitor activity in our standard hNE-inhibition assay.

Both API and SLPI are inactivated by low molar ratios of oxidant to inhibitor. The Chloramine-T:protein molar ratios required for 50% inhibition of API and SLPI are about 1:1 and 2:1, respectively. These ratios correspond well with the reported presence of two and four readily oxidized methionine residues in API and SLPI, respectively. In contrast, all four EPI-HNE proteins retain essentially complete hNE-inhibition activity following exposure to Chloramine-T at all molar ratios tested (up to 50:1, in the cases of EPI-HNE-3 and EPI-HNE-4). Neither EPI-HNE-3 nor EPI-HNE-4 contain any methionine residues. In contrast, EPI-HNE-1 and EPI-HNE-2 each contains two methionine residues (see Table 10). The resistance of these proteins to oxidative inactivation indicates that the methionine residues are either inaccessible to the oxidant or are located in a region of the protein that does not interact with hNE.

### EXAMPLE 18

### pH Stability

Table 38 shows the results of measurements of the pH stability of EPI-HNE proteins. The stability of the proteins to exposure to pH conditions in the range of pH 1 to pH 10 was assessed by maintaining the inhibitors in buffers of defined pH at 37° C. for 18 hours and determining the residual hNE inhibitory activity in the standard hNE-inhibition assay. Proteins were incubated at a concentration of 1 μM. The buffers shown in Table 4 were formulated as described (STOL90) and used in the pH ranges indicated:

TABLE 4
Buffers used in stability studies

| Buffer | Lowest pH | Highest pH |
|---|---|---|
| Glycine-HCl | 1 | 2.99 |
| Citrate-Phosphate | 3 | 7 |
| Phosphate | 7 | 8 |
| Glycine-NaOH | 8.5 | 10 |

Both BPTI-derived inhibitors, EPI-HNE-1 and EPI-HNE-2, are stable at all pH values tested. EPI-HNE-3 and EPI-HNE-4, the inhibitors derived from the human protein Kunitz-type domain, were stable when incubated at low pH, but showed some loss of activity at high pH. When incubated at 37° C. for 18 hours at pH=7.5, the EPI-HNE-3 preparation lost 10 to 15% of its hNE-inhibition activity. EPI-HNE-4 retains almost full activity to pH 8.5. Activity of the ITI-D2-derived inhibitor declined sharply at higher pH levels so that at pH 10 only 30% of the original activity remained. The sensitivity of EPI-HNE-3 to incubation at high pH probably explains the loss of activity of the protein in the final purification step noted previously.

### EXAMPLE 19

### Temperature Stability

The stability of EPI-HNE proteins to temperatures in the range 0° C. to 95° C. was assessed by incubating the inhibitors for thirty minutes at various temperatures and determining residual inhibitory activity for hNE. In these experiments, protein concentrations were 1 μM in phosphate buffer at pH=7. As is shown in Table 40, the four inhibitors are quite temperature stable.

EPI-HNE-1 and EPI-HNE-2 maintain full activity at all temperatures below about 90° C. EPI-HNE-3 and EPI-HNE-4 maintain full inhibitory activity when incubated at temperatures below 65° C. The activity of the protein declines somewhat at higher temperatures. However, all three proteins retain more than ≈50% activity even when incubated at 95° C. for 30 minutes.

### EXAMPLE 20

### Routes to Other hNE-Inhibitory Sequences

The present invention demonstrates that very high-affinity hNE inhibitors can be devised from Kunitz domains of human origin with very few amino-acid substitutions. It is believed that almost any Kunitz domain can be made into a potent and specific hNE inhibitor with eight or fewer substitutions. In particular, any one of the known human Kunitz domains could be remodeled to provide a highly stable, highly potent, and highly selective hNE inhibitor. There are at least three routes to hNE inhibitory Kunitz domains: 1) replacement of segments known to be involved in specifying hNE binding, 2) replacement of single residues thought to be important for hNE binding, and 3) use of libraries of Kunitz domains to select hNE inhibitors.

### EXAMPLE 21

### Substitution of Segments in Kunitz Domains

Table 10 shows the amino-acid sequences of 11 human Kunitz domains. These sequences have been broken into ten segments: 1:N terminus-residue 4; 2:residue 5; 3:6-9(or 9a); 4:10-13; 5:14; 6:15-21; 7:22-30, 8:31-36; 8:37-38; 9:39-42; and 10:43-C terminus (or 42a-C terminus).

Segments 1, 3, 5, 7, and 9 contain residues that strongly influence the binding properties of Kunitz domains and are double underscored in the Consensus Kunitz Domain of Table 10. Other than segment 1, all the segments are the same length except for TFPI-2 Domain 2 which carries an extra residue in segment 2 and two extra residues in segment 10.

Segment 1 is at the amino terminus and influences the binding by affecting the stability and dynamics of the protein. Segments 3, 5, 7, and 9 contain residues that contact serine proteases when a Kunitz domain binds in the active site. High-affinity hNE inhibition requires a molecule that is highly complementary to hNE. Segments 3, 5, 7, and 9 supply the amino acids that contact the protease. The sequences in segments 1, 3, 5, 7, and 9 must work together in the context supplied by each other and the other segments. Nevertheless, we have demonstrated that very many different sequences are capable of high-affinity hNE inhibition.

It may be desirable to have an hNE inhibitor that is highly similar to a human protein to reduce the chance of immunogenicity. Candidate high-affinity hNE inhibitor protein sequences may be obtained by taking an aprotonin-type Kunitz domain that strongly or very strongly inhibits hNE, and replacing one, two, three, four or all of segments 2, 4, 6, 8, and 10 with the corresponding segment from a human Kunitz domain, such as those listed in Table 10, or other domain known to have relatively low immunogenicity in humans. (Each of segments 2, 4, 6, 8, and 10 may be taken from the same human domain, or they may be taken from different human domains.) Alternatively, a reduced immunogenicity, high hNE inhibiting domain may be obtained by taking one of the human aprotonin-type Kunitz domains and replacing one, two, three or all of segments 3, 5, 7 and 9 (and preferably also segment 1) with the corresponding segment from one or more aprotonin-like Kunitz domains that strongly or very strongly inhibit hNE. In making these humanized hNE inhibitors, one may, of course, use, rather than a segment identical to that of one of the aforementioned source proteins, a segment which differs from the native source segment by one or more conservative modifications. Such differences should, of course, be taken with due consideration for their possible effect on inhibitory activity and/or immunogenicity. In some cases, it may be advantageous that the segment be a hybrid of corresponding segments from two or more human domains (in the case of segments 2, 4, 6, 8 and 10) or from two or more strong or very strong hNE inhibitor domains (in the case of segments 3, 5, 7, and 9). Segment 1 may correspond to the segment 1 of a strong or very strong hNE inhibitor, or the segment 1 of a human aprotonin-like Kunitz domain, or be a chimera of segment 1's from both.

The proteins DPI.1.1, DPI.2.1, DPI.3.1, DPI.4.1, DPI.5.1, DPI.6.3, DPI.7.1, DPI.8.1, and DPI.9.1 were designed in this way. DPI.1.1 is derived from App-I by replacing segments 3, 5, 7, and 9 with the corresponding segments from EPI-HNE-1. DPI.2.1 is derived from TFPI2-D1 by replacing segments 3, 5, 7, and 9 with the corresponding residues from EPI-HNE-1. DPI.3.1 is derived from TFPI2-D2 by replacing residues 9a-21 with residues 10-21 of EPI-HNE-4 and replacing residues 31-42b with residues 31-42 of EPI-HNE-4. DPI.4.1 is derived from TFPI2-D3 by replacing segments 3, 5, 7, and 9 with the corresponding residues from MUTQE. DPI.5.1 is derived from LACI-D1 by replacing segments 3, 5, 7, and 9 with the corresponding residues from MUTQE. DPI.6.1 is derived from LACI-D2 by replacing segments 3, 5, 7, and 9 with the corresponding residues from MUTQE. DPI.7.1 is derived from LACI-D3 by replacing segments 3, 5, 7, 9 with the corresponding residues from EPI-HNE-4. DPI.8.1 is derived from the A3 collogen Kunitz domain by substitution of segments 3, 5, 7, and 9 from EPI-HNE-4. DPI.9.1 is derived from the HKI B9 domain by replacing segments 3, 5, 7, and 9 with the corresponding residues from EPI-HNE-4.

While the above-described chimera constitute preferred embodiments of the present invention, the invention is not limited to these chimera.

### EXAMPLE 22

### Point Substitutions in Kunitz Domains

In this example, certain substitution mutations are discussed. It must be emphasized that this example describes preferred embodiments of the invention, and is not intended to limit the invention.

All of the protein sequences mentioned in this example are to be found in Table 10. Designed protease inhibitors are designated “DPI” and are derived from human Kunitz domains (also listed in Table 10). Each of the sequences designated DPI.i.2 (for i=1 to 9) is derived from the domain two above it in the table by making minimal point mutations. Each of the sequences designated DPI.i.3 (for i=1 to 9) is derived from the sequence three above it by more extensive mutations intended to increase affinity. For some parental domains, additional examples are given. The sequences designated DPI.i.1 are discussed in Example 21.

The most important positions are 18 and 15. Any Kunitz domain is likely to become a good hNE inhibitor if Val or Ile is at 15 (with Ile being preferred) and Phe is at 18. (However, these features are not necessarily required for such activity.)

If a Kunitz domain has Phe at 18 and either Ile or Val at 15 and is not a good hNE inhibitor, there may be one or more residues in the interface preventing proper binding.

The Kunitz domains having very high affinity for hNE herein disclosed (as listed in Table 10) have no charged groups at residues 10, 12 through 19, 21, and 32 through 42. At position 11, only neutral and positively charged groups have been observed in very high affinity hNE inhibitors. At position 31, only neutral and negatively charged groups have been observed in high-affinity hNE inhibitors. If a parental Kunitz domain has a charged group at any of those positions where only neutral groups have been observed, then each of the charged groups is preferably changed to an uncharged group picked from the possibilities in Table 46 as the next step in improving binding to hNE. Similarly, negatively charged groups at 11 and 19 and positively charged groups at 31 are preferably replaced by groups picked from Table 46.

At position 10, Tyr, Ser, and Val are seen in high-affinity hNE inhibitors. Asn or Ala may be allowed since this position may not contact hNE. At position 11, Thr, Ala, and Arg have been seen in high-affinity hNE inhibitors. Gln and Pro are very common at 11 in Kunitz domains and may be acceptable. Position 12 is almost always Gly. If 12 is not Gly, try changing it to Gly.

All of the high-affinity hNE inhibitors produced so far have Pro₁₃, but it has not been shown that this is required. Many (62.5%) Kunitz domains have Pro₁₃. If 13 is not Pro, then changing to Pro may improve the hNE affinity. Val, Ala, Leu, or Ile may also be acceptable here.

Position 14 is Cys. It is possible to make domains highly similar to Kunitz domains in which the 14-38 disulfide is omitted. Such domains are likely to be less stable than true Kunitz domains having the three standard disulfides.

Position 15 is preferably Ile or Val. Ile is more preferred.

Most Kunitz domains (82%) have either Gly or Ala at 16 and this may be quite important. If residue 16 is not Gly or Ala, change 16 to either Gly or Ala; Ala is preferred. Position 17 in very potent hNE inhibitors has either Phe or Met; those having Ile or Leu at 17 are less potent. Phe is preferred. Met should be used only if resistance to oxidation is not important. Position 18 is Phe.

It has been shown that high-affinity hNE inhibitors may have either Pro or Ser at position 19. Gln or Lys at position 19 may be allowed. At position 21, Tyr and Trp have been seen in very high affinity hNE inhibitors; Phe may also work.

At position 31, Gln, Glu, and Val have been observed in high affinity hNE inhibitors. Since this is on the edge of the binding interface, other types are likely to work well. One should avoid basic types (Arg and Lys). At position 32, Thr and Leu have been observed in high-affinity hNE inhibitors. This residue may not make direct contact and other uncharged types may work well. Pro is very common here. Ser has been seen and is similar to Thr. Ala has been seen in natural Kunitz domains and is unlikely to make any conflict. Position 33 is always Phe in Kunitz domains.

It appears that many amino acid types may be placed at position 34 while retaining high affinity for hNE; large hydrophobic residues (Phe, Trp, Tyr) are unfavorable. Val and Pro are most preferred at 34. Positions 35-38 contain the sequence Tyr-Gly-Gly-Cys. There is a little diversity at position 36 in natural Kunitz domains. In the BPTI-Trypsin complex, changing Gly₃₆ to Ser greatly reduces the binding to trypsin. Nevertheless, S36 or T36 may not interfere with binding to hNE and could even improve it. If residue 36 is not Gly, one should consider changing it to Gly.

Position 39 seems to tolerate a variety of types. Met and Gln are known to work in very high-affinity inhibitors. Either Ala or Gly are acceptable at position 40; Gly is preferred. At position 41, Asn is by far the most common type in natural Kunitz domains and may act to stabilize the domains. At position 42, Gly is preferred, but Ala is allowed.

Finally, positions that are highly conserved in Kunitz domains may be converted to the conserved type if needed. For example, the mutations X36G, X37G, X41N, and X12G may be desirable in those cases that do not already have these amino acids at these positions.

The above mutations are summarized in Table 41. Table 41 contains, for example, mutations of the form X15I which means change the residue at position 15 (whatever it is) to Ile or leave it alone if it is already Ile. A Kunitz domain that contains the mutation X18F and either X15I or X15V (X15I preferred) will have strong affinity for hNE. As from one up to about 8 of the mutations found in Table 41 are asserted, the affinity of the protein for hNE will increase so that the Kᵢ approaches the range 1-5 pM.

The sequence DPI.1.2 was constructed from the sequence of App-I by the changes R15I, I18F, and F34V and should be a potent hNE inhibitor. DPI.1.3 is likely to be a more potent inhibitor, having the changes R15I, M17F (to avoid sensitivity to oxidation), I18F, P32T, F34V, and G39M.

DPI.2.2 was derived from the sequence of TFPI2-D1 by the changes R15I, L18F, and L34V and should be a potent hNE inhibitor. DPI.2.3 may be more potent due to the changes Y11T, R15I, L17F, L18F, R31Q, Q32T, L34V, and E39M.

DPI.3.2 is derived from TFPI2-D2 by the changes E15I, T18F, S26A(to prevent glycosylation), K32T, and F34V and should be a potent hNE inhibitor. DPI.3.3 may be more potent by having the changes Δ9a, D11A, D12G, Q13P, E15I, S17F, T18F, E19K, K20R, N24A (to prevent glycosylation), K32T, F34V, and Δ42a-42b.

DPI.4.2 is derived from TFPI2-D3 by the changes S15I, N17F, and V18F and should be a potent inhibitor of hNE. DPI.4.3 may be more potent by having the changes E11T, L13P, S15I, N17F, V18F, A32T, T34V, and T36G.

DPI.5.2 is derived from LACI-D1 by the changes K15I and M18F and is likely to be a potent inhibitor of hNE. DPI.5.3 may be more potent due to the changes D10Y, D11T, K15I, I17F, M18F, and E32T. Other changes that may improve DPI.5.3 include F21W, I34V, E39M, and Q42G.

The sequence of DPI.6.2 was constructed from the sequence of human LACI-D2 by the mutations R15V and I18F. The rest of the sequence of LACI-D2 appears to be compatible with hNE binding. DPI.6.3 carries two further mutations that make it more like the hNE inhibitors here disclosed: Y17F and K34V. Other alterations that are likely to improve the hNE binding of LACI-D2 include I13P, R32T, and D10S. DPI.6.4 is derived from DPI.6.3 by the additional alteration N25A that will prevent glycosylation when the protein is produced in a eukaryotic cell. Other substitutions that would prevent glycosylation include: N25K, T27A, T27E, N25S, and N25S. DPI.6.5 moves further toward the ITI-D1, ITI-D2, and BPTI derivatives that are known to have affinity for hNE in the 1-5 pM range through the mutations I13P, R15V, Y17F, I18F, T19Q, N25A, K34V, and L39Q. In DPI.6.6, the T19Q and N25A mutations have been reverted. Thus the protein would be glycosylated in yeast or other eukaryotic cells at N₂₅. DPI.6.7 carries the alterations I13P, R15I, Y17F, I18F, T19P, K34V, and L39Q.

DPI.7.2 is derived from human LACI domain 3 by the mutations R15V and E18F. DPI.7.3 carries the mutations R15V, N17F, E18F, and T46K. The T46K mutation should prevent glycosylation at N44. DPI.7.4 carries more mutations so that it is much more similar to the known high-affinity hNE inhibitors. The mutations are D10V, L13P, R15V, N17F, E18F, K34V, S36G, and T46K. DPI.7.5 carries a different set of alterations: L13P, R15I, N17F, E18F, N19P, F21W, R31Q, P32T, K34V, S36G, and T46K; DPI.7.5 should not be glycosylated in eukaryotic cells.

DPI.8.2 is derived from the sequence of the A3 collagen Kunitz domain by the changes R15I, D16A, I18F, and W34V and is expected to be a potent hNE inhibitor. DPI.8.3 is derived from the A3 collagen Kunitz domain by the changes T13P, R15I, D16A, I18F, K20R, and W34V.

DPI.9.2 is derived from the HKI B9 Kunitz domain by the changes Q15I, T16A, and M18F and is expected to be a potent hNE inhibitor. DPI.9.3 may be more potent due to the changes Q15I, T16A, M18F, T19P, E31V, and A34V.

### EXAMPLE 23

### Libraries of Kunitz Domains

Other Kunitz domains that can potently inhibit hNE may be derived from human Kunitz domains either by substituting hNE-inhibiting sequences into human domains or by using the methods of U.S. Pat. No. 5,223,409 and related patents. Table 42 shows a gene that will cause display of human LACI-D2 on M13 gIIIp; essentially the same gene could be used to achieve display on M13 gVIIIp or other anchor proteins (such as bacterial outer-surface proteins (OSPs)). Table 43 shows a gene to cause display of human LACI D1.

Table 44 and Table 45 give variegations of LACI-D1 and LACI-D2 respectively. Each of these is divided into variegation of residues 10-21 in one segment and residues 31-42 in another. In each case, the appropriate vgDNA is introduced into a vector that displays the parental protein and the library of display phage are fractionated for binding to immobilized hNE.

TABLE 5

BPTI Homologues (1-19)

| | | | | | | | | | | | | | | | | | | | |
|---|---|---|---|---|---|---|---|---|---|---|---|---|---|---|---|---|---|---|---|
| R# | 1 | 2 | 3 | 4 | 5 | 6 | 7 | 8 | 9 | 10 | 11 | 12 | 13 | 14 | 15 | 16 | 17 | 18 | 19 |

| | | | | | | | | | | | | | | | | | | | |
|---|---|---|---|---|---|---|---|---|---|---|---|---|---|---|---|---|---|---|---|
| −3 | - | - | - | F | - | - | - | - | - | - | - | - | - | - | - | - | Z | - | - |
| −2 | - | - | - | Q | T | - | - | - | - | - | - | Q | - | - | - | H | G | Z | - |
| −1 | - | - | - | T | E | - | - | - | - | - | - | P | - | - | - | D | D | G | - |
| 1 | R | R | R | P | R | R | R | R | R | R | R | L | A | R | R | R | K | R | A |
| 2 | P | P | P | P | P | P | P | P | P | P | P | R | A | P | P | P | R | P | A |
| 3 | D | D | D | D | D | D | D | D | D | D | D | K | K | D | R | T | D | S | K |
| 4 | F | F | F | L | F | F | F | F | F | F | F | L | Y | F | F | F | I | F | Y |
| 5 | C | C | C | C | C | C | C | C | C | C | C | C | C | C | C | C | C | C | C |
| 6 | L | L | L | Q | L | L | L | L | L | L | L | I | K | E | E | N | R | N | K |
| 7 | E | E | E | L | E | E | E | E | E | E | E | L | L | L | L | L | L | L | L |
| 8 | P | P | P | P | P | P | P | P | P | P | P | H | P | P | P | P | P | P | P |
| 9 | P | P | P | Q | P | P | P | P | P | P | P | R | L | A | A | P | P | A | V |
| 10 | Y | Y | Y | A | Y | Y | Y | Y | Y | Y | Y | N | R | E | E | E | E | E | R |
| 11 | T | T | T | R | T | T | T | T | T | T | T | P | I | T | T | S | Q | T | Y |
| 12 | G | G | G | G | G | G | G | G | G | G | G | G | G | G | G | G | G | G | |
| 13 | P | P | P | P | P | P | P | P | P | P | P | R | P | L | L | R | P | P | P |
| 14 | C | T | A | C | C | C | C | C | C | C | C | C | C | C | C | C | C | C | C |
| 15 | K | K | K | K | K | V | G | A | L | I | K | Y | K | K | K | R | K | K | K |
| 16 | A | A | A | A | A | A | A | A | A | A | A | Q | R | A | A | G | G | A | K |
| 17 | R | R | R | A | A | R | R | R | R | R | R | K | K | Y | R | H | R | S | K |
| 18 | I | I | I | L | M | I | I | I | I | I | I | I | I | I | I | I | L | I | F |
| 19 | I | I | I | L | I | I | I | I | I | I | I | P | P | R | R | R | P | R | P |
| 20 | R | R | R | R | R | R | R | R | R | R | R | A | S | S | S | R | R | Q | S |
| 21 | Y | Y | Y | Y | Y | Y | Y | Y | Y | Y | Y | F | F | F | F | I | Y | Y | F |
| 22 | F | F | F | F | F | F | F | F | F | F | F | Y | Y | H | H | Y | F | Y | Y |
| 23 | Y | Y | Y | Y | Y | Y | Y | Y | Y | Y | Y | Y | Y | Y | Y | Y | Y | Y | Y |
| 24 | N | N | N | N | N | N | N | N | N | N | N | N | K | N | N | N | N | N | N |
| 25 | A | A | A | S | A | A | A | A | A | A | A | Q | W | L | R | L | P | S | W |
| 26 | K | K | K | T | K | K | K | K | K | K | K | K | K | A | A | E | A | K | K |
| 27 | A | A | A | S | A | A | A | A | A | A | A | K | A | A | A | S | S | S | A |
| 28 | G | G | G | N | G | G | G | G | G | G | G | K | K | Q | Q | N | R | G | K |
| 29 | L | L | L | A | F | L | L | L | L | L | L | Q | Q | Q | Q | K | M | G | Q |
| 30 | C | C | C | C | C | C | C | C | C | C | C | C | C | C | C | C | C | C | C |
| 31 | Q | Q | Q | E | E | Q | Q | Q | Q | Q | Q | E | L | L | L | K | E | Q | L |
| 32 | T | T | T | P | T | T | T | T | T | T | T | G | P | Q | E | V | S | Q | P |
| 33 | F | F | F | F | F | F | F | F | F | F | F | F | F | F | F | F | F | F | F |
| 34 | V | V | V | T | V | V | V | V | V | V | V | T | D | I | I | F | I | I | N |
| 35 | Y | Y | Y | Y | Y | Y | Y | Y | Y | Y | Y | W | Y | Y | Y | Y | Y | Y | Y |
| 36 | G | G | G | G | G | G | G | G | G | G | G | S | S | G | G | G | G | G | S |
| 37 | G | G | G | G | G | G | G | G | G | G | G | G | G | G | G | G | G | G | G |
| 38 | C | T | A | C | C | C | C | C | C | C | C | C | C | C | C | C | C | C | C |
| 39 | R | R | R | Q | R | R | R | R | R | R | R | G | G | G | G | G | K | R | G |
| 40 | A | A | A | G | A | A | A | A | A | A | A | G | G | G | G | G | G | G | G |
| 41 | K | K | K | N | K | K | K | K | K | K | K | N | N | N | N | N | N | N | N |
| 42 | R | R | R | N | S | R | R | R | R | R | R | S | A | A | A | A | K | Q | A |
| 43 | N | N | N | N | N | N | N | N | N | N | N | N | N | N | N | N | N | N | N |
| 44 | N | N | N | N | N | N | N | N | N | N | N | R | R | R | R | N | N | R | R |
| 45 | F | F | F | F | F | F | F | F | F | F | F | F | F | F | F | F | F | F | F |
| 46 | K | K | K | E | K | K | K | K | K | K | K | K | K | K | K | E | K | D | K |
| 47 | S | S | S | T | S | S | S | S | S | S | S | T | T | T | T | T | T | T | T |
| 48 | A | A | A | T | A | A | A | A | A | A | A | I | I | I | I | R | K | T | I |
| 49 | E | E | E | E | E | E | E | E | E | E | E | E | E | D | D | D | A | Q | E |
| 50 | D | D | D | M | D | D | D | D | D | D | D | E | E | E | E | E | E | Q | E |
| 51 | C | C | C | C | C | C | C | C | C | C | C | C | C | C | C | C | C | C | C |
| 52 | M | M | M | L | M | M | M | M | M | M | E | R | R | R | H | R | V | Q | R |
| 53 | R | R | R | R | R | R | R | R | R | R | R | R | R | R | R | E | R | G | R |
| 54 | T | T | T | I | T | T | T | T | T | T | T | T | T | T | T | T | A | V | T |
| 55 | C | C | C | C | C | C | C | C | C | C | C | C | C | C | C | C | C | C | C |
| 56 | G | G | G | E | G | G | G | G | G | G | G | I | V | V | V | G | R | V | V |
| 57 | G | G | G | P | G | G | G | G | G | G | G | R | G | G | G | G | P | - | G |
| 58 | A | A | A | P | A | A | A | A | A | A | A | K | - | - | - | K | P | - | - |
| 59 | - | - | - | Q | - | - | - | - | - | - | - | - | - | - | - | - | E | - | - |
| 60 | - | - | - | Q | - | - | - | - | - | - | - | - | - | - | - | - | R | - | - |
| 61 | - | - | - | T | - | - | - | - | - | - | - | - | - | - | - | - | P | - | - |
| 62 | - | - | - | D | - | - | - | - | - | - | - | - | - | - | - | - | - | - | - |
| 63 | - | - | - | K | - | - | - | - | - | - | - | - | - | - | - | - | - | - | - |
| 64 | - | - | - | S | - | - | - | - | - | - | - | - | - | - | - | - | - | - | - |

(BPTI Homologues 20-35)

| | | | | | | | | | | | | | | | | | | | | | |
|---|---|---|---|---|---|---|---|---|---|---|---|---|---|---|---|---|---|---|---|---|---|
| R# | 20 | 21 | 22 | 23 | 24 | 25 | 26 | 27 | 28 | 29 | 30 | 31 | 32 | 33 | 34 | 35 | 36 | 37 | 38 | 39 | 40 |

| | | | | | | | | | | | | | | | | | | | | | |
|---|---|---|---|---|---|---|---|---|---|---|---|---|---|---|---|---|---|---|---|---|---|
| −5 | - | - | - | - | - | - | - | - | - | - | - | - | - | D | - | - | - | - | - | - | - |
| −4 | - | - | - | - | - | - | - | - | - | - | - | - | - | E | - | - | - | - | - | - | - |
| −3 | - | - | - | - | - | - | - | - | - | - | - | - | T | P | - | - | - | - | - | - | - |
| −2 | Z | - | L | Z | R | K | - | - | - | R | R | - | E | T | - | - | - | - | - | - | - |
| −1 | P | - | Q | D | D | N | - | - | - | Q | K | - | R | T | - | - | - | Z | - | - | - |
| 1 | R | R | H | H | R | R | I | K | T | R | R | R | G | D | K | T | R | R | R | R | R |
| 2 | R | P | R | P | P | P | N | E | V | H | H | P | F | L | A | V | P | P | P | P | P |
| 3 | K | Y | T | K | K | T | G | D | A | R | P | D | L | P | D | E | D | D | D | D | D |
| 4 | L | A | F | F | F | F | D | S | A | D | D | F | D | I | S | A | F | F | F | F | F |
| 5 | C | C | C | C | C | C | C | C | C | C | C | C | C | C | C | C | C | C | C | C | C |
| 6 | I | E | K | Y | Y | N | E | Q | N | D | D | L | T | E | Q | N | L | L | L | L | L |
| 7 | L | L | L | L | L | L | L | L | L | K | K | E | S | Q | L | L | E | E | E | E | E |
| 8 | H | I | P | P | P | L | P | G | P | P | P | P | P | A | D | P | P | P | P | P | P |
| 9 | R | V | A | A | A | P | K | Y | V | P | P | P | P | FG | Y | I | P | P | P | P | P |
| 10 | N | A | E | D | D | E | V | S | I | D | D | Y | V | D | S | V | Y | Y | Y | Y | Y |
| 11 | P | A | P | P | P | T | V | A | R | K | T | T | T | A | Q | Q | T | T | T | T | T |
| 12 | G | G | G | G | G | G | G | G | G | G | K | G | G | G | G | G | G | G | G | G | G |
| 13 | R | P | P | R | R | R | P | P | P | N | I | P | P | L | P | P | P | P | P | P | P |
| 14 | C | C | C | C | C | C | C | C | C | C | C | C | C | C | C | C | C | C | C | C | C |
| 15 | Y | M | K | K | L | N | R | M | R | - | - | K | R | F | L | R | R | K | K | K | K |
| 16 | D | F | A | A | A | A | A | G | A | G | Q | A | A | G | G | A | A | A | A | A | A |
| 17 | K | F | S | H | Y | L | R | M | F | P | T | K | G | Y | L | F | R | R | R | R | K |
| 18 | I | I | I | I | M | I | F | T | I | V | V | M | F | M | F | I | I | M | I | M | M |
| 19 | P | S | P | P | P | P | P | S | Q | R | R | I | K | K | K | Q | I | I | I | I | I |
| 20 | A | A | A | R | R | A | R | R | L | A | A | R | R | L | R | L | R | R | R | R | R |
| 21 | F | F | F | F | F | F | Y | Y | W | F | F | Y | Y | Y | Y | W | Y | Y | Y | Y | Y |
| 22 | Y | Y | Y | Y | Y | Y | Y | F | A | Y | Y | F | N | S | F | A | F | F | F | F | F |
| 23 | Y | Y | Y | Y | Y | Y | Y | Y | F | Y | Y | Y | Y | Y | Y | F | Y | Y | Y | Y | Y |
| 24 | N | S | N | D | N | N | N | N | D | D | K | N | N | N | N | D | N | N | N | N | N |
| 25 | Q | K | W | S | P | S | S | G | A | T | P | A | T | Q | G | A | A | A | A | A | A |
| 26 | K | G | A | A | A | H | S | T | V | R | S | K | R | E | T | V | K | K | K | K | K |
| 27 | K | A | A | S | S | L | S | S | K | L | A | A | T | T | S | K | A | A | A | A | A |
| 28 | K | N | K | N | N | H | K | M | G | K | K | G | K | K | M | G | G | G | G | G | G |
| 29 | Q | K | K | K | K | K | R | A | K | T | R | F | Q | N | A | K | L | L | L | L | F |
| 30 | C | C | C | C | C | C | C | C | C | C | C | C | C | C | C | C | C | C | C | C | C |
| 31 | E | Y | Q | N | E | Q | E | E | V | K | V | E | E | E | E | V | Q | Q | Q | Q | E |
| 32 | R | P | L | K | K | K | K | T | L | A | Q | T | P | E | T | R | T | P | P | P | T |
| 33 | F | F | F | F | F | F | F | F | F | F | F | F | F | F | F | F | F | F | F | F | F |
| 34 | D | T | H | I | I | N | I | Q | P | Q | R | V | K | I | L | S | V | V | V | V | V |
| 35 | W | Y | Y | Y | Y | Y | Y | Y | Y | Y | Y | Y | Y | Y | Y | Y | Y | Y | Y | Y | Y |
| 36 | S | S | G | G | G | G | G | G | G | R | G | G | G | G | G | G | G | G | G | G | G |
| 37 | G | G | G | G | G | G | G | G | G | G | G | G | G | G | G | G | G | G | G | G | G |
| 38 | C | C | C | C | C | C | C | C | C | C | C | C | C | C | C | C | C | C | C | C | C |
| 39 | G | R | K | P | R | G | G | M | Q | D | D | K | K | Q | M | K | R | R | R | R | K |
| 40 | G | G | G | G | G | G | G | G | G | G | G | A | G | G | G | G | A | A | A | A | A |
| 41 | N | N | N | N | N | N | N | N | N | D | D | K | N | N | N | N | K | K | K | K | K |
| 42 | S | A | A | A | A | A | A | G | G | H | H | S | G | D | L | G | R | S | R | R | S |
| 43 | N | N | N | N | N | N | N | N | N | G | G | N | N | N | N | N | N | N | N | N | N |
| 44 | R | R | R | N | N | N | N | N | K | N | N | N | R | R | N | K | N | N | N | N | N |
| 45 | F | F | F | F | F | F | F | F | F | F | F | F | Y | F | F | F | F | F | F | F | F |
| 46 | K | K | S | K | K | K | H | V | Y | K | K | R | K | S | L | Y | K | K | K | K | R |
| 47 | T | T | T | T | T | T | T | T | S | T | S | S | S | T | S | S | S | S | S | S | S |
| 48 | I | I | I | W | W | I | L | E | E | E | D | A | E | L | Q | Q | A | A | S | A | A |
| 49 | E | E | E | D | D | D | E | K | K | T | H | E | Q | A | K | K | E | E | E | E | E |
| 50 | E | E | K | E | E | E | E | E | E | L | L | D | D | E | E | E | D | D | D | D | D |
| 51 | C | C | C | C | C | C | C | C | C | C | C | C | C | C | C | C | C | C | C | C | C |
| 52 | R | R | R | R | R | Q | E | L | R | R | R | M | L | E | L | K | E | M | M | M | M |
| 53 | R | R | H | Q | H | R | K | Q | E | C | C | R | D | Q | Q | E | R | R | R | R | R |
| 54 | T | T | A | T | T | T | V | T | Y | E | E | T | A | K | T | Y | T | T | T | T | T |
| 55 | C | C | C | C | C | C | C | C | C | C | C | C | C | C | C | C | C | C | C | C | C |
| 56 | I | V | V | G | V | A | G | R | G | L | E | G | S | I | R | G | G | G | G | G | G |
| 57 | G | V | G | A | A | A | V | - | V | V | L | G | G | N | - | I | G | G | G | G | G |
| 58 | - | - | - | S | S | K | R | - | P | Y | Y | A | F | - | - | P | A | A | A | A | A |
| 59 | - | - | - | A | G | Y | S | - | G | P | R | - | - | - | - | G | - | - | - | - | - |
| 60 | - | - | - | - | I | G | - | - | D | - | - | - | - | - | - | E | - | - | - | - | - |
| 61 | - | - | - | - | - | - | - | - | E | - | - | - | - | - | - | A | - | - | - | - | - |

### Legend to Table 5

1 BPTI SEQ ID NO:87

2 Engineered BPTI From MARK87 SEQ ID NO:88

3 Engineered BPTI From MARK87 SEQ ID NO:89

4 Bovine Colostrum (DUFT85) SEQ ID NO:90

5 Bovine Serum (DUFT85) SEQ ID NO:91

6 Semisynthetic BPTI, TSCH87 SEQ ID NO:92

7 Semisynthetic BPTI, TSCH87 SEQ ID NO:93

8 Semisynthetic BPTI, TSCH87 SEQ ID NO:94

9 Semisynthetic BPTI, TSCH87 SEQ ID NO:95

10 Semisynthetic BPTI, TSCH87 SEQ ID NO:96

11 Engineered BPTI, AUER87 SEQ ID NO:97

12 Dendroaspis polylepis polylepis (Black mamba) venom I(DUFT85) SEQ ID NO:98

13 Dendroaspis polylepis polylepis (Black Mamba) venom K DUFT85) SEQ ID NO:99

14 Hemachatus hemachates (Ringhals Cobra) HHV II (DUFT85) SEQ ID NO:100

15 Naja nivea (Cape cobra) NNV II (DUFT85) SEQ ID NO:101

16 Vipera russelli (Russel's viper) RW II (TAKA74) SEQ ID NO:102

17 Red sea turtle egg white (DUFT85) SEQ ID NO:103

18 Snail mucus (Helix pomania) (WAGN78) SEQ ID NO:104

19 Dendroaspis angusticeps (Eastern green mamba) C13 S1 C3 toxin (DUFT85) SEQ ID NO:105

20 Dendroaspis angusticeps (Eastern Green Mamba) C13 S2 C3 toxin (DUFT85) SEQ ID NO:106

21 Dendroaspis polylepis polylepes (Black mamba) B toxin (DUFT85) SEQ ID NO:107

22 Dendroaspis polylepis polylepes (Black Mamba) E toxin (DUFT85) SEQ ID NO:108

23 Vipera ammodytes TI toxin (DUFT85) SEQ ID NO:109

24 Vipera ammodytes CTI toxin (DUFT85) SEQ ID NO:110

25 Bungarus fasciatus VIII B toxin (DUFT85) SEQ ID NO:111

26 Anemonia sulcata (sea anemone) 5 II (DUFT85) SEQ ID NO:112

27 Homo sapiens HI-8e “inactive” domain (DUFT85) SEQ ID NO:113

28 Homo sapiens HI-8“active” domain (DUFT85) SEQ ID NO:114

29 beta bungarotoxin B1 (DUFT85) SEQ ID NO:115

30 beta bungarotoxin B2 (DUFT85) SEQ ID NO:116

31 Bovine spleen TI II (FIOR85) SEQ ID NO:117

32 Tachypleus tridentatus (Horseshoe crab) hemocyte inhibitor (NAKA87) SEQ ID NO:118

33 Bombyx mori (silkworm) SCI-III (SASA84) SEQ ID NO:119

34 Bos taurus (inactive) BI-14 SEQ ID NO:120

35 Bos taurus (active) BI-8 SEQ ID NO:121

36: Engineered BPTI (KR15, ME52) SEQ ID NO:122: Auerswald '88, Biol Chem Hoppe-Seyler, 369 Supplement, pp 27-35.

37: Isoaprotinin G-1 SEQ ID NO:123: Siekmann, Wenzel, Schroder, and Tschesche '88, Biol Chem Hoppe-Seyler, 369:157-163.

38: Isoaprotinin 2 SEQ ID NO:124: Siekmann, Wenzel, Schroder, and Tschesche '88, Biol Chem Hoppe-Seyler, 369:157-163.

39: Isoaprotinin G-2 SEQ ID NO:125: Siekmann, Wenzel, Schroder, and Tschesche '88, Biol Chem Hoppe-Seyler, 369:157-163.

40: Isoaprotinin 1 SEQ ID NO:126: Siekmann, Wenzel, Schroder, and Tschesche '88, Biol Chem Hoppe-Seyler, 369:157-163.

Notes:

a) both beta bungarotoxins have residue 15 deleted.
b) B. mori has an extra residue between C5 and C14; we have assigned F and G to residue 9.
c) all natural proteins have C at 5, 14, 30, 38, 50, & 55.
d) all homologues have F33 and G37.

e) extra C's in bungarotoxins form interchain cystine bridges

TABLE 6

Tables
IIIsp::bpti::matureIII(initial fragment) fusion gene. The DNA sequence
has SEQ ID NO. 001; Amino-acid sequence has SEQ ID NO. 002. The DNA is
linear and is shown on the lines that do not begin with “!”. The DNA encoding
mature III is identical to the DNA found in M13mp18. The amino-acid sequence is
processed in vivo and disulfide bonds form.

```
! SEQ ID NO. 002 m k k l l f a I p l
! 1 2 3 4 5 6 7 8 9 10
    SEQ ID NO. 001 5′-gtg aaa aaa tta tta ttc gca att cct tta
! |<---- gene III signal peptide -------
!
! -cleavage site
! ↓
! v v p f y s G A
! 11 12 13 14 15 16 17 18
        gtt gtt cct ttc tat tct GGc Gcc
! --------------------------->|
!
! |R|P|D|F|C|L|E|
! |19|20|21|22|23|24|25|
                |CGT|CCG|GAT|TTC|TGT|CTC|GAG|-
! M13/BPTI Jnct ‘↑’ |AccIII| |XhoI | (& AvaI)!
!
! |P|P|Y|T|G|P|C|K|A|R|
! |26|27|28|29|30|31|32|33|34|35|
  |CCA|CCA|TAC|ACT|GGG|CCC|TGC|AAA|GCG|CGC|-
! | PflMI | || |BssHII|
! |ApαI ||
! |DrαII | = PssI
!
!|I|I|R|Y|F|Y|N|A|K|A|
!|36|37|38|39|40|41|42|43|44|45|
  |ATC|ATC|CGC|TAT|TTC|TAC|AAT|GCT|AAA|GC|-
!
!|G|L|C|Q|T|F|V|Y|G|G|
!|46|47|48|49|50|51|52|53|54|55|
  A|GGC|CTG|TGC|CAG|ACC|TTT|GTA|TAC|GGT|GGT|-
!|StuI| |XcαI |(& AccI)
!
!|C|R|A|K|R|N|N|F|K|
!|56|57|58|59|60|61|62|63|64|
  |TGC|CGT|GCT|AAG|CGT|AAC|AAC|TTT|AAA|-
! |EspI |
!
!|S|A|E|D|C|M|R|T|C|G|
!|65|66|67|68|69|70|71|72|73|74|
  |TCG|GCC|GAA|GAT|TGC|ATG|CGT|ACC|TGC|GGT|-
! |XmaIII| |SphI |
!
! BPTI/M13 boundary
! ↓
!|G|A|A E (Residue numbers of mature III have had
!|75|76|119 120 118 added to the usual residue numbers.)
  |GGC|GCC|gct gaa-
!|NarI | (& KasI)
!
! 121 122 123 124 125 126 127 128 129 130 131 132 133 134
! T V E S C L A K P H T E N S . . .
  act gtt gaa agt tgt tta gca aaa ccc cat aca gaa aat tca . . .
!
!The remainder of the gene is identical to the corresponding part of iii in M13 mp18.
```

TABLE 7

IIIsp::itiD1::matureIII fusion gene.
DNA has SEQ ID NO. 003; amino-acid sequence has SEQ ID NO. 004.
The DNA is a linear segment and the amino-acid sequence is a protein that is
processed in vivo and which contains disulfides.

```
SEQ ID NO. 004
m k k l l f a I p l v v p f y
−18 −17 −16 −15 −14 −13 −12 −11 −10 −9 −8 −7 −6 −5 −4
5′-gtg aaa aaa tta tta ttc gca att cct tta gtt gtt cct ttc tat
SEQ ID NO. 003
|<---- gene III signal peptide -----------------------------
                  -cleavage site
                  |
s G A K E D S C Q L G Y S A G
−3 −2 −1 1 2 3 4 5 6 7 8 9 10 11 12
tct GGc Gcc aaa gaa gaC tcT tGC CAG CTG GGC tac tCG GCC Ggt
------->| | BglI | |EagI|
    |KasI|
13 14 15 16 17 18 19 20 21 22 23 24 25 26
P C M G M T S R Y F Y N G T
ccc tgc atg gga atg acc agc agg tat ttc tat aat ggt aca
27 28 29 30 31 32 33 34 35 36 37 38 39 40 41
S M A C E T F Q Y G G C M G N
tCC ATG Gcc tgt gag act ttc cag tac ggc ggc tgc atg ggc aac
|NcoI|
|StyI|
42 43 44 45 46 47 48 49 50 51 52 53 54 55 56
G N N F V T E K E C L Q T C R
ggt aac aac ttc gtc aca gaa aag gag tgt CTG CAG acc tgc cga
                                                  |PstI |
57 58 101 102 119 120
T V g a A E
act gtg ggc gcc gct gaa
            |BbeI | (Residue numbers of mature
            |NarI | III have had 118 added to
            |KasI | the usual residue numbers.)
121 122 123 124 125 126 127 128 129 130 131 132 133 134 135
  T V E S C L A K P H T E N S F . .
act gtt gaa agt tgt tta gca aaa ccc cat aca gaa aat tca ttt . .
The remainder of the gene is identical to the corresponding part of gene iii in phage
M13mp18.
```

TABLE 8
Affinity Classes of ITI-D1-derived hNE inhibitors

| | | Fraction of | | |
|---|---|---|---|---|
| Affinity | Estimated | Input | pH Elution | |
| Class | K_{D} | bound | Maximum | Protein |
| WEAK | K_{D} >10 | <0.005% | >6.0 | ITI-D1 |
| | nM | | | |
| MODERATE | 1 to 10 | 0.01% to | 5.5 to 5.0 | BITI |
| | nM | 0.03% | | ITI-D1E7 |
| STRONG | 10 to 1000 | 0.03% to | 5.0 to 4.5 | BITI-E7 |
| | pM | 0.06% | | BITI-E7-1222 |
| | | | | AMINO1 |
| | | | | AMINO2 |
| | | | | MUTP1 |
| VERY | <10 pM | >0.1% | ≦4.0 | BITI-E7-141 |
| STRONG | | | | MUTT26A |
| | | | | MUTQE |
| | | | | MUT1619 |

TABLE 9
Definition of Class A, B and C mutations in PCT/US92/01501.

| Res. | | | |
|---|---|---|---|
| Id. | EpiNE1 | Substitutions | Class |
| 1 | R | any | A |
| 2 | P | any | A |
| 3 | D | any | A |
| 4 | F | Y, W, L | B |
| 5 | C | C | X |
| 6 | L | non-proline | A |
| 7 | E | L, S, T, D, N, K, R | A |
| 8 | P | any | A |
| 9 | P | any | A |
| 10 | Y | non-proline prefr'd | B |
| 11 | T | any | C |
| 12 | G | must be G | X |
| 13 | P | any | C |
| 14 | C | C strongly preferred, any non-proline | C |
| 15 | I | V, A | C |
| 16 | A | | C |
| 17 | F | L, I, M, Y, W, H, V | C |
| 18 | F | Y, W, H | C |
| 19 | P | any | C |
| 20 | R | non-proline prefr'd | C |
| 21 | Y | F & Y most prefr'd; W, I, L prefr'd; M, V | C |
| | | allowed | |
| 22 | F | Y & F most prefr'd; non-proline prefr'd | Y, F B |
| 23 | Y | Y & F strongly prefr'd | F, Y B |
| 24 | N | non-proline prefr'd | A |
| 25 | A | any | A |
| 26 | K | any | A |
| 27 | A | any | A |
| 28 | G | non-proline prefr'd | A |
| 29 | L | non-proline prefr'd | A |
| 30 | C | must be C | X |
| 31 | Q | non-proline prefr'd | B |
| 32 | T | non-proline prefr'd | B |
| 33 | F | F very strongly prefr'd; Y possible | X |
| 34 | V | any | C |
| 35 | Y | Y most prefr'd; W prefr'd; F allowed | B |
| 36 | G | G strongly prefr'd; S, A prefr'd; | C |
| 37 | G | must be G so long as 38 is C | X |
| 38 | C | C strongly prefr'd | X |
| 39 | M | any | C |
| 40 | G | A, S, N, D, T, P | C |
| 41 | N | K, Q, S, D, R, T, A, E | C |
| 42 | G | any | C |
| 43 | N | must be N | X |
| 44 | N | S, K, R, T, Q, D, E | B |
| 45 | F | Y | B |
| 46 | K | any non-proline | B |
| 47 | S | T, N, A, G | B |
| 48 | A | any | B |
| 49 | E | any | A |
| 50 | D | any | A |
| 51 | C | must be C | X |
| 52 | M | any | A |
| 53 | R | any | A |
| 54 | T | any | A |
| 55 | C | must be C | X |
| 56 | G | any | A |
| 57 | G | any | A |
| 58 | A | any | A |

prefr'd stands for preferred.
Classes:
A No major effect expected if molecular charge stays in range −1 to +1.
B Major effects not expected, but are more likely than in “A”.
C Residue in the binding interface; any change must be tested.
X No substitution allowed.

TABLE 10

Sequences of Kunitz domains

| | Sequence | | Seq |
|---|---|---|---|
| | 111111111122222222223333333333444 4444444555555555 | Parental | Id |
| Name | 123456789a012345678901234567890123456789012ab3456799012345678 | domain | No. |
| Consensus | RPDFCLLPA-ETGPCRAMIPRFYYNAKSGKCEPFIYGGCGGNA--NNFKTEEECRRTCGGA | | 005 |
| Kunitz | 1 3 5 7 9 | | |
| Domain | 2 4 6 8 10 | | |
| BPTI | RPDFCLEPP-YTGPCKARIIRYFYNAKAGLCQTFVYGGCRAKR-- | BPTI | 006 |
| (Genebank | NNFKSAEDCMRTCGGA | | |
| P00974) | | | |
| EPI- | rpdfclepp-ytgpcIaFFPryfynakaglcqtfvyggcMGNG--nnfksaedcmrtcgga | BPTI | 007 |
| HNE-1 = | | | |
| EpiNE1 | | | |
| EPI-HNE-2 | EAEArpdfclepp-ytgpcIaFFPryfynakaglcqtfvyggcMGNG--nnfksaedcmrtcgga | BPTI | 008 |
| EpiNE7 | rpdfclepp-ytgpcVaMFPryfynakaglcqtfvyggcMGNG--nnfksaedcmrtcgga | BPTI | 009 |
| EpiNE3 | rpdfclepp-ytgpcVGFFSryfynakaglcqtfvyggcMGNG--nnfksaedcmrtcgga | BPTI | 010 |
| EpiNE6 | rpdfclepp-ytgpcVGFFQryfynakaglcqtfvyggcMGNG--nnfksaedcmrtcgga | BPTI | 011 |
| EpiNE4 | rpdfclepp-ytgpcVaIFPryfynakaglcqtfvyggcMGNG--nnfksaedcmrtcgga | BPTI | 012 |
| EpiNE8 | rpdfclepp-ytgpcVaFFKrsfynakaglcqtfvyggcMGNG--nnfksaedcmrtcgga | BPTI | 013 |
| EpiNE5 | rpdfclepp-ytgpcIaFFQryfynakaglcqtfvyggcMGNG--nnfksaedcmrtcgga | BPTI | 014 |
| EpiNE2 | rpdfclepp-ytgpcIaLFKryfynakaglcqtfvyggcMGNG--nnfksaedcmrtcgga | BPTI | 015 |
| ITI-D1 | KEDSCQLGY-SAGPCMGMTSRYFYNGTSMACETFQYGGCMGNG--NNFVTEKDCLQTCRTV | ITI-D1 | 016 |
| (Genebank | | | |
| P02760) | | | |
| BITI- | RPdFcqlgy-sagpcVAmFPryfyngtsmacQtfVyggcmgng--nnfvtekdclqtcrga | ITI-D1 | 017 |
| E7-141 | | | |
| MUTT26A | RPdFcqlgy-sagpcVAmFPryfyngAsmacQtfVyggcmgng--nnfvtekdclqtcrga | ITI-D1 | 018 |
| MUTQE | RPdFcqlgy-sagpcVAmFPryfyngtsmacetfVyggcmgng--nnfvtekdclqtcrga | ITI-D1 | 019 |
| MUT1619 | RpdFcqlgy-sagpcVgmFsryfyngtsmacQtfVyggcmgng--nnfvtekdclqtcrga | IDI-D1 | 020 |
| ITI-D1E7 | kedscqlgy-sagpcVAmFPryfyngtsmacetfqyggcmgng--nnfvtekdclqtcrga | ITI-D1 | 021 |
| AMINO1 | kedFcqlgy-sagpcVAmFPryfyngtsmacetfqyggcmgng--nnfvtekdclqtcrga | ITI-D1 | 022 |
| AMINO2 | kPdscqlgy-sagpcVAmFPryfyngtsmacetfqyggcmgng--nnfvtekdclqtcrga | ITI-D1 | 023 |
| MUTP1 | RPdFcqlgy-sagpcIgmFsryfyngtsmacetfqyggcmgng--nnfvtekdclqtcrga | ITI-D1 | 024 |
| ITI-D2 | TVAACNLPI-VRGPCRAFIQLWAFDAVKGKCVLFPYGGCQGNG-- | ITI-D2 | 025 |
| (Genebank | NKFYSEKECREYCGVP | | |
| P02760) | | | |
| EPI-HNE-3 | aacnlpi-vrgpcIafFPRwafdavkgkcvlfpyggcqgng--nkfysekecreycgvp | ITI-D2 | 026 |
| EPI-HNE-4 | Eacnlpi-vrgpcIafFPRwafdavkgkcvlfpyggcqgng--nkfysekecreycgvp | ITI-D2 | 027 |
| App-I | VREVCSEQA-ETGPCRAMISRWYFDVTEGKCAPFFYGGCGGNR--NNFDTEEYCMAVCGSA | | 028 |
| (NCBI | | | |
| 105306) | | | |
| DPI.1.1 | vrevcseqa-YtgpcIaFFPrYyfdvtegkcQTfVyggcMgnG--nnfdteeycmavcgsa | APP-I | 029 |
| DPI.1.2 | vrevcseqa-etgpcIamFsrwyfdvtegkcapfVyggcggnr--nnfdteeycmavcgsa | App-I | 030 |
| DPI.1.3 | vrevcseqa-etgpcIaFFsrwyfdvtegkcaTfVyggcMgnr--nnfdteeycmavcgsa | App-I | 031 |
| TFPI2-D1 | NAEICLLPL-DYGPCRALLLRYYYDRYTQSCRQFLYGGCEGNA-- | | 032 |
| (SPRE94) | NNFYTWEACDDACWRI | | |
| DPI.2.1 | naeicllpl-YTgpcIaFFPryyydrytqscQTfVyggcMgna--nnfytweacddacwri | TFPI2-D1 | 033 |
| DPI.2.2 | naeicllpl-dygpcIalFlryyydrytqscrqfVyggcegna--nnfytweacddacwri | TFPI2-D1 | 034 |
| DPI.2.3 | naeicllpl-dTgpcIaFFlryyydrytqscQTfVyggcMgna--nnfytweacddacwri | TFPI2-D1 | 035 |
| TFPI2-D2 | VPKVCRLQVSVDDQCEGSTEKYFFNLSSMTCEKFFSGGCHRNRIENRFPDEATCMGFCAPK | | 036 |
| (SPRE94) | | | |
| DPI.3.1 | vpkvcrlqv-vRGPcIAFFPRWffnlssmtcVLfPYggcQGnG--nrfpdeatcmgfcapk | | 037 |
| DPI.3.2 | vpkvcrlqvsvddqcIgsFekyffnlAsmtceTfVsggchrnrienrfpdeatcmgfcapk | TFPI2-D2 | 038 |
| DPI.3.3 | vpkvcrlqv-vAGPcIgFFKRyffAlssmtceTfVsggchrnr--nrfpdeatcmgfcapk | TFPI2-D2 | 039 |
| TFPI2-D3 | ipsfcyspk-deglcsanvtryyfnpryrtcdaftytgcggnd--nnfvsredckracaka | | 040 |
| (SPRE94) | | | |
| DPI.4.1 | ipsfcyspk-SAgPcVaMFPryyfnpryrtcETfVyGgcMgnG--nnfvsredckracaka | TFPI2-D3 | 041 |
| DPI.4.2 | ipsfcyspk-deglcIaFFtryyfnpryrtcdaftytgcggnd--nnfvsredckracaka | TFPI2-D3 | 042 |
| DPI.4.3 | ipsfcyspk-dTgPcIaFFtryyfnpryrtcdTfVyGgcggnd--nnfvsredckracaka | TFPI2-D3 | 043 |
| LACI-D1 | mhsfcafka-ddgpckaimkrfffniftrqceefiyggcegnq--nrfesleeckkmctrd | | 044 |
| (Genebank | | | |
| P10646) | | | |
| DPI.5.1 | mhsfcafka-SAgpcVaMFPrYffniftrqceTfVyggcMgnG--nrfesleeckkmctrd | LACI-D1 | 045 |
| DPI.5.2 | mhsfcafka-ddgpcIaiFkrfffniftrqceefiyggcegnq--nrfesleeckkmctrd | LACI-D1 | 046 |
| DPI.5.3 | mhsfcafka-YTgpcIaFFkrfffniftrqceTfiyggcegnq--nrfesleeckkmctrd | LACI-D1 | 047 |
| LACI-D2 | KPDFCFLEE-DPGICRGYITRYFYNNQTKQCERFKYGCCLGNM--NNFETLEECKNICEDG | | 048 |
| (Genebank | | | |
| P10646) | | | |
| DPI.6.1 | kpdfcflee-SAgPcVAMFPryfynnqtkqceTfVyggcMgnG--nnfetleecknicedg | LACI-D2 | 049 |
| DPI.6.2 | kpdfcflee-dpgicVgyFtryfynnqtkqcerfkyggclgnm--nnfetleecknicedg | LACI-D2 | 050 |
| DPI.6.3 | kpdfcflee-dpgicVgFFtryfynnqtkqcerfVyggclgnm--nnfetleecknicedg | LACI-D2 | 051 |
| DPI.6.4 | kpdfcflee-dpgicVgFFtryfynAqtkqcerfVyggclgnm--nnfetleecknicedg | LACI-D2 | 052 |
| DPI.6.5 | kpdfcflee-dpgPcVgFFQryfynAqtkqcerfVyggcQgnm--nnfetleecknicedg | LACI-D2 | 053 |
| DPI.6.6 | kpdfcflee-dpgPcVgFFtryfynnqtkqcerfVyggcQgnm--nnfetleecknicedg | LACI-D2 | 054 |
| DPI.6.7 | kpdfcflee-dpgPcIgFFPryfynnqtkqcerfVyggcQgnm--nnfetleecknicedg | LACI-D2 | 055 |
| LACI-D3 | GPSWCLTPA-DRGLCRANENRFYYNSVIGKCRPFKYSGCGGNE-- | | 056 |
| (Genebank | NNFTSKQECLRACKKG | | |
| P10646) | | | |
| DPI.7.1 | gpswcltpa-VrgPcIaFFPrWyynsvigkcVLfPyGgcQgnG--nnftskqeclrackkg | LACI-D3 | 057 |
| DPI.7.2 | gpswcltpa-drglcVanFnrfyynsvigkcrpfkysgcggne--nnftskqeclrackkg | LACI-D3 | 058 |
| DPI.7.3 | gpswcltpa-drglcVaFFnrfyynsvigkcrpfkysgcggne--nnfKskqeclrackkg | LACI-D3 | 059 |
| DPI.7.4 | gpswcltpa-VrgPcVaFFnrfyynsvigkcrpfkyGgcggne--nnfKskqeclrackkg | LACI-D3 | 060 |
| DPI.7.5 | gpswcltpa-drgPcIaFFPrWyynsvigkcQTfVyGgcggne--nnfAskqeclrackkg | LACI-D3 | 061 |
| A3 | ETDICKLPK-DEGTCRDFILKWYYDPNTKSCARFWYGGCGGNE-- | | 062 |
| collagen | NKFGSQKECEKVCAPV | | |
| (WO93/ | | | |
| 14119) | | | |
| DPI.8.1 | etdicklpk-VRgPcIAfFPRwyydpntkscVLfPyggcQgnG--nkfgsqkecekvcapv | A3 | 063 |
| DPI.8.2 | etdicklpk-degtcIAfFlkwyydpntkscarfVyggcggne--nkfgsqkecekvcapv | A3 | 064 |
| | | collagen | |
| DPI.8.3 | etdicklpk-degPcIAfFlRwyydpntkscarfVyggcggne--nkfgsqkecekvcapv | A3 | 065 |
| HKI B9 | LPNVCAFPM-EKGPCQTYMTRWFFNFETGECELFAYGGCGGNS-- | | 066 |
| Domain | NNFLRKEKCEKFCKFT | | |
| (NORR93) | | | |
| DPI.9.1 | lpnvcafpm-VRgpcIAFFPrwffnfetgecVlfVyggcQgnG--nnflrkekcekfckft | HKI B9 | 067 |
| DPI.9.2 | lpnvcafpm-ekgpcIAyFtrwffnfetgecelfayggcggns--nnflrkekcekfckft | HKI B9 | 068 |
| DPI.9.3 | lpnvcafpm-ekgpcIAyFPrwffnfetgecVlfVyggcggns--nnflrkekcekfckft | HKI B9 | 069 |

Sequences listed in Table 10 that strongly inhibit hNE are EPI-HNE-1(=EpiNE1), EPI-HNE-2, EpiNE7, EpiNE3, EpiNE6, EpiNE4, EpiNE8, EpiNE5, EpiNE2, BITI-E7-141, MUTT26A, MUTQE, MUT1619, ITI-D1E7, AMINO1, AMINO2, MUTP1, and EPI-HNE-3, and EPI-HNE-4.
Sequences listed in Table 10 that are highly likely to strongly inhibit hNE are DPI.1.1, DPI.1.2, DPI.1.3, DPI.2.1, DPI.2.2, DPI.2.3, DPI.3.1, DPI.3.2, DPI.3.3, DPI.4.1, DPI.4.2, DPI.4.3, DPI.5.1, DPI.5.2, DPI.5.3, DPI.6.1, DPI.6.2, DPI.6.3, DPI.6.4, DPI.6.5, DPI.6.6, DPI.6.7, DPI.7.1, DPI.7.2, DPI.7.3, DPI.7.4, DPI.7.5, DPI.8.1, DPI.8.2, DPI.8.3, DPI.9.1, DPI.9.2, and DPI.9.3.
Human Kunitz domains listed in Table 10: ITI-D1, ITI-D2, App-I, TFPI2-D1, TFPI2-D2, TFPI2-D3, LACI-D1, LACI-D2, LACI-D3, A3 collagen Kunitz domain, and HKI B9 Domain.

TABLE 11

Restriction sites in plasmid pHIL-D2
pHIL-D2, 93-01-02 Ngene = 8157

Non-cutters

| | | | | | | |
|---|---|---|---|---|---|---|
| AflII | ApaI | AscI | AvaI | AvrII | BamHI | BglII |
| Bsp120I | BsrGI | BssHII | BstEII | FseI | MluI | NruI |
| PacI | PmlI | RsrII | SacII | SexAI | SfiI | SgfI |
| SnaBI | SpeI | Sse8387I | XhoI | | XmaI | |
| | | | (PaeR7I) | | (SmaI) | |

| | | |
|---|---|---|
| Cutters | | |
| AatII GACGTc | 1 | 5498 |
| AflIII Acrygt | 1 | 7746 |
| AgeI Accggt | 1 | 1009 |
| BlpI GCtnagc | 1 | 597 |
| BspEI (BspMII, AccIII) Tccgga | 1 | 3551 |
| BspMI gcaggt | 1 | 4140 |
| Bst1107I GTAtac | 1 | 7975 |
| BstBI (AsuII) TTcgaa | 2 | 945 4780 |
| Bsu36I CCtnagg | 1 | 1796 |
| Ec1136I GAGctc | 1 | 216 |
| EcoRI Gaattc | 1 | 956 |
| EspI (Bpu1102I) GCtnagc | 1 | 597 |
| HpaI GTTaac | 1 | 1845 |
| NcoI Ccatgg | 1 | 3339 |
| NdeI CAtatg | 1 | 7924 |
| NsiI (Ppu10I) ATGCAt | 1 | 684 |
| PflMI CCANNNNntgg | 1 | 196 |
| PmeI GTTTaaac | 1 | 420 |
| PstI CTGCAg | 1 | 6175 |
| PvuI CGATcg | 1 | 6049 |
| SapI gaagagc | 1 | 7863 |
| SacI GAGCTc | 1 | 216 |
| SalI Gtcgac | 1 | 2885 |
| ScaI AGTact | 1 | 5938 |
| SphI GCATGc | 1 | 4436 |
| StuI AGGcct | 1 | 2968 |
| SwaI ATTTaaat | 1 | 6532 |
| Tth111I GACNnngtc | 1 | 7999 |
| XbaI Tctaga | 1 | 1741 |
| XcmI CCANNNNNnnnntgg | 1 | 711 |

| | |
|---|---|
| Aox1 5′ | 1 to about 950 |
| Aox1 3′ | 950 to about 1250 |
| His4 | 1700 to about 4200 |
| Aox1 3′ | 4500 to 5400 |
| bla | 5600 to 6400 |
| f1 ori | 6500 to 6900 |

TABLES 12-13 (merged)

SEQUENCES OF THE EpiNE CLONES IN THE P1 REGION

| CLONE | |
|---|---|
| IDENTIFIERS | SEQUENCE |
| | 1 1 1 1 1 1 1 2 2 |
| | 3 4 5 6 7 8 9 0 1 |
| BPTI (comp. only) | P C K A R I I R Y (BPTI) |
| | (SEQ ID NO: 132) |
| | P C V A M F Q R Y EpiNEα |
| | (SEQ ID NO: 132) |
| 3, 9, 16, 17, 18, 19 | P C V G F F S R Y EpiNE3 |
| | (SEQ ID NO: 133) |
| 6 | P C V G F F Q R Y EpiNE6 |
| | (SEQ ID NO: 134) |
| 7, 13, 14, 15, 20 | P C V A M F P R Y EpiNE7 |
| | (SEQ ID NO: 135) |
| 4 | P C V A I F P R Y EpiNE4 |
| | (SEQ ID NO: 136) |
| 8 | P C V A I F K R S EpiNE8 |
| | (SEQ ID NO: 137) |
| 1, 10, 11, 12 | P C I A F F P R Y EpiNE1 |
| | (SEQ ID NO: 138) |
| 5 | P C I A F F Q R Y EpiNE5 |
| | (SEQ ID NO: 139) |
| 2 | P C I A L F K R Y EpiNE2 |
| | (SEQ ID NO: 140) |

Note:
The DNA sequences encoding these amino acid sequences are set forth in 08/133,031, previously incorporated by reference.

TABLE 14
Fractionation of EpiNE-7 and MA-ITI-D1 phage on hNE beads

| | | EpiNE-7 | | MA-ITI-D1 | |
|---|---|---|---|---|---|
| | | pfu | pfu/INPUT | pfu | pfu/INPUT |
| INPUT | | 3.3 · 10⁹ | 1.00 | 3.4 · 10¹¹ | 1.00 |
| Final | | 3.8 · 10⁵ | 1.2 · 10⁻⁴ | 1.8 · 10⁶ | 5.3 · 10⁻⁶ |
| TBS-TWEEN | | | | | |
| Wash | | | | | |
| pH | 7.0 | 6.2 · 10⁵ | 1.8 · 10⁻⁴ | 1.6 · 10⁶ | 4.7 · 10⁻⁶ |
| | 6.0 | 1.4 · 10⁶ | 4.1 · 10⁻⁴ | 1.0 · 10⁶ | 2.9 · 10⁻⁶ |
| | 5.5 | 9.4 · 10⁵ | 2.8 · 10⁻⁴ | 1.6 · 10⁶ | 4.7 · 10⁻⁶ |
| | 5.0 | 9.5 · 10⁵ | 2.9 · 10⁻⁴ | 3.1 · 10⁵ | 9.1 · 10⁻⁷ |
| | 4.5 | 1.2 · 10⁶ | 3.5 · 10⁻⁴ | 1.2 · 10⁵ | 3.5 · 10⁻⁷ |
| | 4.0 | 1.6 · 10⁶ | 4.8 · 10⁻⁴ | 7.2 · 10⁴ | 2.1 · 10⁻⁷ |
| | 3.5 | 9.5 · 10⁵ | 2.9 · 10⁻⁴ | 4.9 · 10⁴ | 1.4 · 10⁻⁷ |
| | 3.0 | 6.6 · 10⁵ | 2.0 · 10⁻⁴ | 2.9 · 10⁴ | 8.5 · 10⁻⁸ |
| | 2.5 | 1.6 · 10⁵ | 4.8 · 10⁻⁵ | 1.4 · 10⁴ | 4.1 · 10⁻⁸ |
| | 2.0 | 3.0 · 10⁵ | 9.1 · 10⁻⁵ | 1.7 · 10⁴ | 5.0 · 10⁻⁸ |

| | | | | |
|---|---|---|---|---|
| SUM | 6.4 · 10⁶ | 3 · 10⁻³ | 5.7 · 10⁶ | 2 · 10⁻⁵ |

* SUM is the total pfu (or fraction of input) obtained from all pH elution fractions

TABLE 15
Abbreviated fractionation of display phage on hNE beads

| | Display phage | | | |
|---|---|---|---|---|
| | EpiNE-7 | MA-ITI-D12 | MA-ITI-D1E71 | MA-ITI-D1E72 |
| INPUT | 1.00 | 1.00 | 1.00 | 1.00 |
| (pfu) | (1.8 × 10⁹) | (1.2 × 10¹⁰ | (3.3 × 10⁹) | (1.1 × 10⁹) |
| Wash | 6 · 10⁻⁵ | 1 · 10⁻⁵ | 2 · 10⁻⁵ | 2 · 10⁻⁵ |
| pH 7.0 | 3 · 10⁻⁴ | 1 · 10⁻⁵ | 2 · 10⁻⁵ | 4 · 10⁻⁵ |
| pH 3.5 | 3 · 10⁻³ | 3 · 10⁻⁶ | 8 · 10⁻⁵ | 8 · 10⁻⁵ |
| pH 2.0 | 1 · 10⁻³ | 1 · 10⁻⁶ | 6 · 10⁻⁶ | 2 · 10⁻⁵ |
| SUM | 4.3 · 10⁻³ | 1.4 · 10⁻⁵ | 1.1 · 10⁻⁴ | 1.4 · 10⁻⁴ |

Each entry is the fraction of input obtained in that component.
SUM is the total fraction of input pfu obtained from all pH elution fractions

TABLE 16
Fractionation of EpiNE-7 and MA-ITI-D1E7 phage on hNE beads

| | EpiNE-7 | | MA-ITI-D1E7 | |
|---|---|---|---|---|
| | | Fraction of | | Fraction of |
| | Total pfu | Input | Total pfu | Input |
| INPUT | 1.8 · 10⁹ | 1.00 | 3.0 · 10⁹ | 1.00 |
| pH 7.0 | 5.2 · 10⁵ | 2.9 · 10⁻⁴ | 6.4 · 10⁴ | 2.1 · 10⁻⁵ |
| pH 6.0 | 6.4 · 10⁵ | 3.6 · 10⁻⁴ | 4.5 · 10⁴ | 1.5 · 10⁻⁵ |
| pH 5.5 | 7.8 · 10⁵ | 4.3 · 10⁻⁴ | 5.0 · 10⁴ | 1.7 · 10⁻⁵ |
| pH 5.0 | 8.4 · 10⁵ | 4.7 · 10⁻⁴ | 5.2 · 10⁴ | 1.7 · 10⁻⁵ |
| pH 4.5 | 1.1 · 10⁶ | 6.1 · 10⁻⁴ | 4.4 · 10⁴ | 1.5 · 10⁻⁵ |
| pH 4.0 | 1.7 · 10⁶ | 9.4 · 10⁻⁴ | 2.6 · 10⁴ | 8.7 · 10⁻⁶ |
| pH 3.5 | 1.1 · 10⁶ | 6.1 · 10⁻⁴ | 1.3 · 10⁴ | 4.3 · 10⁻⁶ |
| pH 3.0 | 3.8 · 10⁵ | 2.1 · 10⁻⁴ | 5.6 · 10³ | 1.9 · 10⁻⁶ |
| pH 2.5 | 2.8 · 10⁵ | 1.6 · 10⁻⁴ | 4.9 · 10³ | 1.6 · 10⁻⁶ |
| pH 2.0 | 2.9 · 10⁵ | 1.6 · 10⁻⁴ | 2.2 · 10³ | 7.3 · 10⁻⁷ |
| SUM | 7.6 · 10⁶ | 4.1 · 10⁻³ | 3.1 · 10⁵ | 1.1 · 10⁻⁴ |

* SUM is the total pfu (or fraction of input) obtained from all pH elution fractions.

TABLE 17
Fractionation of MA-EpiNE-7, MA-BITI and MA-BITI-E7 on hNE beads

| | | MA-BITI | | MA-BITI-E7 | | MA-EpiNE7 | |
|---|---|---|---|---|---|---|---|
| | | pfu | pfu/Input | pfu | pfu/Input | pfu | pfu/Input |
| INPUT | | 2.0 · 10¹⁰ | 1.00 | 6.0 · 10⁹ | 1.00 | 1.5 · 10⁹ | 1.00 |
| pH | 7.0 | 2.4 · 10⁵ | 1.2 · 10⁻⁵ | 2.8 · 10⁵ | 4.7 · 10⁻⁵ | 2.9 · 10⁵ | 1.9 · 10⁻⁴ |
| | 6.0 | 2.5 · 10⁵ | 1.2 · 10⁻⁵ | 2.8 · 10⁵ | 4.7 · 10⁻⁵ | 3.7 · 10⁵ | 2.5 · 10⁻⁴ |
| | 5.0 | 9.6 · 10⁴ | 4.8 · 10⁻⁶ | 3.7 · 10⁵ | 6.2 · 10⁻⁵ | 4.9 · 10⁵ | 3.3 · 10⁻⁴ |
| | 4.5 | 4.4 · 10⁴ | 2.2 · 10⁻⁶ | 3.8 · 10⁵ | 6.3 · 10⁻⁵ | 6.0 · 10⁵ | 4.0 · 10⁻⁴ |
| | 4.0 | 3.1 · 10⁴ | 1.6 · 10⁻⁶ | 2.4 · 10⁵ | 4.0 · 10⁻⁵ | 6.4 · 10⁵ | 4.3 · 10⁻⁴ |
| | 3.5 | 8.6 · 10⁴ | 4.3 · 10⁻⁶ | 9.0 · 10⁴ | 1.5 · 10⁻⁵ | 5.0 · 10⁵ | 3.3 · 10⁻⁴ |
| | 3.0 | 2.2 · 10⁴ | 1.1 · 10⁻⁶ | 8.9 · 10⁴ | 1.5 · 10⁻⁵ | 1.9 · 10⁵ | 1.3 · 10⁻⁴ |
| | 2.5 | 2.2 · 10⁴ | 1.1 · 10⁻⁶ | 2.3 · 10⁴ | 3.8 · 10⁻⁶ | 7.7 · 10⁴ | 5.1 · 10⁻⁵ |
| | 2.0 | 7.7 · 10³ | 3.8 · 10⁻⁷ | 8.7 · 10³ | 1.4 · 10⁻⁶ | 9.7 · 10⁴ | 6.5 · 10⁻⁵ |
| SUM | | 8.0 · 10⁵ | 3.9 · 10⁻⁵ | 1.8 · 10⁶ | 2.9 · 10⁻⁴ | 3.3 · 10⁶ | 2.2 · 10⁻³ |

* SUM is the total pfu (or fraction of input) obtained from all pH elution fractions

TABLE 18
Fractionation of MA-BITI-E7 and MA-BITI-E7-1222 on hNE beads

| | | MA-BITI-E7 | | MA-BITI-E7-1222 | |
|---|---|---|---|---|---|
| | | pfu | pfu/INPUT | pfu | pfu/INPUT |
| INPUT | | 1.3 · 10⁹ | 1.00 | 1.2 · 10⁹ | 1.00 |
| pH | 7.0 | 4.7 · 10⁴ | 3.6 · 10⁻⁵ | 4.0 · 10⁴ | 3.3 · 10⁻⁵ |
| | 6.0 | 5.3 · 10⁴ | 4.1 · 10⁻⁵ | 5.5 · 10⁴ | 4.6 · 10⁻⁵ |
| | 5.5 | 7.1 · 10⁴ | 5.5 · 10⁻⁵ | 5.4 · 10⁴ | 4.5 · 10⁻⁵ |
| | 5.0 | 9.0 · 10⁴ | 6.9 · 10⁻⁵ | 6.7 · 10⁴ | 5.6 · 10⁻⁵ |
| | 4.5 | 6.2 · 10⁴ | 4.8 · 10⁻⁵ | 6.7 · 10⁴ | 5.6 · 10⁻⁵ |
| | 4.0 | 3.4 · 10⁴ | 2.6 · 10⁻⁵ | 2.7 · 10⁴ | 2.2 · 10⁻⁵ |
| | 3.5 | 1.8 · 10⁴ | 1.4 · 10⁻⁵ | 2.3 · 10⁴ | 1.9 · 10⁻⁵ |
| | 3.0 | 2.5 · 10³ | 1.9 · 10⁻⁶ | 6.3 · 10³ | 5.2 · 10⁻⁶ |
| | 2.5 | <1.3 · 10³ | <1.0 · 10⁻⁶ | <1.3 · 10³ | <1.0 · 10⁻⁶ |
| | 2.0 | 1.3 · 10³ | 1.0 · 10⁻⁶ | 1.3 · 10³ | 1.0 · 10⁻⁶ |

| | | | | |
|---|---|---|---|---|
| SUM | 3.8 · 10⁵ | 2.9 · 10⁻⁴ | 3.4 · 10⁵ | 2.8 · 10⁻⁴ |

SUM is the total pfu (or fraction of input) obtained from all pH elution fractions

TABLE 19
Fractionation of MA-EpiNE7 and MA-BITI-E7-141 on hNE beads

| | | MA-EpiNE7 | | MA-BITI-E7-141 | |
|---|---|---|---|---|---|
| | | pfu | pfu/INPUT | pfu | pfu/INPUT |
| INPUT | | 6.1 · 10⁸ | 1.00 | 2.0 · 10⁹ | 1.00 |
| pH | 7.0 | 5.3 · 10⁴ | 8.7 · 10⁻⁵ | 4.5 · 10⁵ | 2.2 · 10⁻⁴ |
| | 6.0 | 9.7 · 10⁴ | 1.6 · 10⁻⁴ | 4.4 · 10⁵ | 2.2 · 10⁻⁴ |
| | 5.5 | 1.1 · 10⁵ | 1.8 · 10⁻⁴ | 4.4 · 10⁵ | 2.2 · 10⁻⁴ |
| | 5.0 | 1.4 · 10⁵ | 2.3 · 10⁻⁴ | 7.2 · 10⁵ | 3.6 · 10⁻⁴ |
| | 4.5 | 1.0 · 10⁵ | 1.6 · 10⁻⁴ | 1.3 · 10⁶ | 6.5 · 10⁻⁴ |
| | 4.0 | 2.0 · 10⁵ | 3.3 · 10⁻⁴ | 1.1 · 10⁶ | 5.5 · 10⁻⁴ |
| | 3.5 | 9.7 · 10⁴ | 1.6 · 10⁻⁴ | 5.9 · 10⁵ | 3.0 · 10⁻⁴ |
| | 3.0 | 3.8 · 10⁴ | 6.2 · 10⁻⁵ | 2.3 · 10⁵ | 1.2 · 10⁻⁴ |
| | 2.5 | 1.3 · 10⁴ | 2.1 · 10⁻⁵ | 1.2 · 10⁵ | 6.0 · 10⁻⁵ |
| | 2.0 | 1.6 · 10⁴ | 2.6 · 10⁻⁵ | 1.0 · 10⁵ | 5.0 · 10⁻⁵ |

| | | | | |
|---|---|---|---|---|
| SUM | 8.6 · 10⁵ | 1.4 · 10⁻³ | 5.5 · 10⁶ | 2.8 · 10⁻³ |

SUM is the total pfu (or fraction of input) obtained from all pH elution fractions.

TABLE 20
pH Elution Analysis of hNE Binding by BITI-E7-141
Varient Display Phage

| | | Fraction of Input | | | | |
|---|---|---|---|---|---|---|
| | Input | recovered at pH | | | Recovery | |
| | PFU | | pH3.5 | pH2.0 | Total | |
| Displayed protein | (×10⁹) | pH7.0 | ×10⁻⁴ | ×10⁻⁴ | ×10⁻⁴ | Relative |
| AMINO1 (EE) | 0.96 | 0.24 | 2.3 | 0.35 | 2.9 | 0.11 |
| AMINO2 (AE) | 6.1 | 0.57 | 2.1 | 0.45 | 3.1 | 0.12 |
| BITI-E7-1222 | 1.2 | 0.72 | 4.0 | 0.64 | 5.4 | 0.21 |
| (EE) | | | | | | |
| EpiNE7 (EE) | 0.72 | 0.44 | 6.4 | 2.2 | 9.0 | 0.35 |
| MUTP1 (AE) | 3.9 | 1.8 | 9.2 | 1.2 | 12.0 | 0.46 |
| MUT1619 (EE) | 0.78 | 0.82 | 9.9 | 0.84 | 12.0 | 0.46 |
| MUTQE (AE) | 4.7 | 1.2 | 16. | 5.3 | 22.0 | 0.85 |
| MUTT26A (EE) | 0.51 | 2.5 | 19.0 | 3.3 | 25.0 | 0.96 |
| BITI-E7-141 | 1.7 | 2.2 | 18.0 | 5.4 | 26.0 | 1.00 |
| (AE) | | | | | | |
| BITI-E7-141 (EE) | 0.75 | 2.1 | 21. | 3.2 | 26.0 | 1.00 |

Notes:
EE Extended pH elution protocol
AE Abbreviated pH elution protocol
Total Total fraction of input = Sum of fractions collected at pH 7.0, pH 3.5, and pH 2.0.
Relative Total fraction of input recovered divided by total fraction of input recovered for BITI-E7-141

TABLE 21
ITI-D1-derived hNE Inhibitors

WEAK (K_{D > 10}⁻⁸ M)

| | |
|---|---|
| | 1 1 2 2 3 3 4 4 5 5 |
| | 1...5....0....5.....0...5....0....5....0....5....0....5... |
| 1. | KEDSCQLGYSAGPCMGMTSRYFYNGTSMACETFQYGGCMGNGNNFVTEKDCLQTCRGA |

MODERATE (10⁻⁸ > RD 22 10⁻⁹)

| | |
|---|---|
| 2. | KEDSCQLGYSAGPCVAMFPRYFYNGTSMACETFQYGGCMGNGNNFVTEKDCLQTCRGA |
| 3. | RPDFCQLGYSAGPCMGMTSRYFYNGTSMACETFQYGGCMGNGNNFVTEKDCLQTCRGA |

STRONG (10⁻⁹ > KD > 10⁻¹¹ D)

| | |
|---|---|
| 4. | RPDFCQLGYSAGPCVAMFPRYFYNGTSMACETFQYGGCMGNGNNFVTEKDCLQTCRGA |
| 5. | RPDFCQLGYSTGPCVAMFPRYFYNGTSMACETFQYGGCMGNGNNFVTEKDCLQTCRGA |
| 6. | KEDFCQLGYSAGPCVAMFPRYFYNGTSMACETFQYGGCMGNGNNFVTEKDCLQTCRGA |
| 7. | KPDSCQLGYSAGPCVAMFPRYFYNGTSMACETFQYGGCMGNGNNFVTEKDCLQTCRGA |
| 8. | RPDFCQLGYSAGPCIGMFSRYFYNGTSMACETFQYGGCMGNGNNFVTEKDCLQTCRGA |

VERY STRONG (K_{D} < 10⁻¹¹ M)

| | |
|---|---|
| 9. | RPDFCQLGYSAGPCVAMFPRYFYNGTSMACQTFVYGGCMGNGNNFVTEKDCLQTCRGA |
| 10. | RPDFCQLGYSAGPCVAMFPRYFYNGASMACQTFVYGGCMGNGNNFVTEKDCLQTCRGA |
| 11. | RPDFCQLGYSAGPCVAMFPRYFYNGTSMACETFVYGGCMGNGNNFVTEKDCLQTCRGA |
| 12. | RPDFCQLGYSAGPCVGMFSRYFYNGTSMACQTFVYGGCMGNGNNFVTEKDCLQTCRGA |

Residues shown underlined and bold are changed from those present in ITID1
Sequences Key:
1. ITI-D1 SEQ ID NO. 16
2. ITI-D1E7 SEQ ID NO. 21
3. BITI SEQ ID NO. 141
4. BITI-E7 SEQ ID NO. 142
5. BITI-E7-1222 SEQ ID NO. 143
6. AMINO1 SEQ ID NO. 22
7. AMINO2 SEQ ID NO. 23
8. MUTP1 SEQ ID NO. 24
9. BITI-E7-141 SEQ ID NO. 17
10. MUTT26A SEQ ID NO. 18
11 MUTQE SEQ ID NO. 19
12 MUT1619 SEQ ID NO. 20

TABLE 22

Same sequences as in Table 21 showing only changes (and
Cysteines for alignment).

WEAK (K_{D} > 10⁻⁸ M)

| | |
|---|---|
| | 1 1 2 2 3 3 4 4 5 5 |
| | 1...5....0....5.....0...5....0....5....0....5....0....5... |
| 1. | KEDSCQLGYSAGPCMGMTSRYFYNGTSMACETFQYGGCMGNGNNFVTEKDCLQTCRGA |

MODERATE (10⁻⁸ > RD 22 10⁻⁹)

| | |
|---|---|
| 2. | ---C--------CVA-FP----------C-------C------------C---C--- |
| 3. | RP--C--------C---------------C-------C------------C---C--- |

STRONG (10⁻⁹ > KD > 10⁻¹¹D)

| | |
|---|---|
| 4. | RP--C--------CVA-FP----------C-------C------------C---C--- |
| 5. | RP--C-----T--CVA-FP----------C-------C------------C---C--- |
| 6. | ---FC--------CVA-FP----------C-------C------------C---C--- |
| 7. | -P--C--------CVA-FP----------C-------C------------C---C--- |
| 8. | RP-FC--------CI--FP----------C-------C------------C---C--- |

VERY STRONG (K_{D} < 10⁻¹¹ M)

| | |
|---|---|
| 9. | RP-FC--------CVA-FP----------CQ--V---C------------C---C--- |
| 10. | RP-FC--------CVA-FP------A---CQ--V---C------------C---C--- |
| 11. | RP-FC--------CVA-FP----------C---V---C------------C---C--- |
| 12. | RP-FC--------CV---F----------CQ--V---C------------C---C--- |

Residues shown underlined and bold are changed from those present in ITID1.

TABLE 23

Plasmid pHIL-D2 SEQ ID NO. 070
8157 base pairs. Only one strand is shown, but the
DNA exists as double-stranded circular DNA in
vivo.

| | |
|---|---|
| | 1 2 3 4 5 |
| | 1234567890 1234567890 1234567890 1234567890 |
| | 1234567890 |
| 1 | AgATCgCggC CgCgATCTAA CATCCAAAgA CgAAAggTTg |
| | AATgAAACCT |
| 51 | TTTTgCCATC CgACATCCAC AggTCCATTC TCACACATAA |
| | gTgCCAAACg |
| 101 | CAACAggAgg ggATACACTA gCAgCAgACC gTTgCAAACg |
| | CAggACCTCC |
| 151 | ACTCCTCTTC TCCTCAACAC CCACTTTTgC CATCgAAAAA |
| | CCAgCCCAgT |
| 201 | TATTgggCTT gATTggAgCT CgCTCATTCC AATTCCTTCT |
| | ATTAggCTAC |
| 251 | TAACACCATg ACTTTATTAg CCTgTCTATC CTggCCCCCC |
| | TggCgAggTC |
| 301 | ATgTTTgTTT ATTTCCgAAT gCAACAAgCT CCgCATTACA |
| | CCCgAACATC |
| 351 | ACTCCAgATg AgggCTTTCT gAgTgTgggg TCAAATAgTT |
| | TCATgTTCCC |
| 401 | AAATggCCCA AAACTgACAg TTTAAACgCT gTCTTggAAC |
| | CTAATATgAC |
| 451 | AAAAgCgTgA TCTCATCCAA gATgAACTAA gTTTggTTCg |
| | TTgAAATgCT |
| 501 | AACggCCAgT TggTCAAAAA gAAACTTCCA AAAgTCgCCA |
| | TACCgTTTgT |
| 551 | CTTgTTTggT ATTgATTgAC gAATgCTCAA AAATAATCTC |
| | ATTAATgCTT |
| 601 | AgCgCAgTCT CTCTATCgCT TCTgAACCCg gTggCACCTg |
| | TgCCgAAACg |
| 651 | CAAATggggA AACAACCCgC TTTTTggATg ATTATgCATT |
| | gTCCTCCACA |
| 701 | TTgTATgCTT CCAAgATTCT ggTgggAATA CTgCTgATAg |
| | CCTAACgTTC |
| 751 | ATgATCAAAA TTTAACTgTT CTAACCCCTA CTTgACAggC |
| | AATATATAAA |
| 801 | CAgAAggAAg CTgCCCTgTC TTAAACCTTT TTTTTTATCA |
| | TCATTATTAg |
| 851 | CTTACTTTCA TAATTgCgAC TggTTCCAAT TgACAAgCTT |
| | TTgATTTTAA |
| 901 | CgACTTTTAA CgACAACTTg AgAAgATCAA AAAACAACTA |
| | ATTATTCgAA |
| | BstBI |
| 951 | ACgAggAATT CgCCTTAgAC ATgACTgTTC CTCAgTTCAA |
| | gTTgggCATT |
| | EcoRI |
| 1001 | ACgAgAAgAC CggTCTTgCT AgATTCTAAT CAAgAggATg |
| | TCAgAATgCC |
| 1051 | ATTTgCCTgA gAgATgCAgg CTTCATTTTT gATACTTTTT |
| | TATTTgTAAC |
| 1101 | CTATATAgTA TAggATTTTT TTTgTCATTT TgTTTCTTCT |
| | CgTACgAgCT |
| 1151 | TgCTCCTgAT CAgCCTATCT CgCAgCTgAT gAATATCTTg |
| | TggTAggggT |
| 1201 | TTgggAAAAT CATTCgAgTT TgATgTTTTT CTTggTATTT |
| | CCCACTCCTC |
| 1251 | TTCAgAgTAC AgAAgATTAA gTgAgAAgTT CgTTTgTgCA |
| | AgCTTATCgA |
| 1301 | TAAgCTTTAA TgCggTAgTT TATCACAgTT AAATTgCTAA |
| | CgCAgTCAgg |
| 1351 | CACCgTgTAT gAAATCTAAC AATgCgCTCA TCgTCATCCT |
| | CggCACCgTC |
| 1401 | ACCCTggATg CTgTAggCAT AggCTTggTT ATgCCggTAC |
| | TgCCgggCCT |
| 1451 | CTTgCgggAT ATCgTCCATT CCgACAgCAT CgCCAgTCAC |
| | TATggCgTgC |
| 1501 | TgCTAgCgCT ATATgCgTTg ATgCAATTTC TATgCgCACC |
| | CgTTCTCggA |
| 1551 | gCACTgTCCg ACCgCTTTgg CCgCCgCCCA gTCCTgCTCg |
| | CTTCgCTACT |
| 1601 | TggAgCCACT ATCgACTACg CgATCATggC gACCACACCC |
| | gTCCTgTggA |
| 1651 | TCTATCgAAT CTAAATgTAA gTTAAAATCT CTAAATAATT |
| | AAATAAgTCC |
| 1701 | CAgTTTCTCC ATACgAACCT TAACAgCATT gCggTgAgCA |
| | TCTAgACCTT |
| 1751 | CAACAgCAgC CAgATCCATC ACTgCTTggC CAATATgTTT |
| | CAgTCCCTCA |
| 1801 | ggAgTTACgT CTTgTgAAgT gATgAACTTC TggAAggTTg |
| | CAgTgTTAAC |
| 1851 | TCCgCTgTAT TgACgggCAT ATCCgTACgT TggCAAAgTg |
| | TggTTggTAC |
| 1901 | CggAggAgTA ATCTCCACAA CTCTCTggAg AgTAggCACC |
| | AACAAACACA |
| 1951 | gATCCAgCgT gTTgTACTTg ATCAACATAA gAAgAAgCAT |
| | TCTCgATTTg |
| 2001 | CAggATCAAg TgTTCAggAg CgTACTgATT ggACATTTCC |
| | AAAgCCTgCT |
| 2051 | CgTAggTTgC AACCgATAgg gTTgTAgAgT gTgCAATACA |
| | CTTgCgTACA |
| 2101 | ATTTCAACCC TTggCAACTg CACAgCTTgg TTgTgAACAg |
| | CATCTTCAAT |
| 2151 | TCTggCAAgC TCCTTgTCTg TCATATCgAC AgCCAACAgA |
| | ATCACCTggg |
| 2201 | AATCAATACC ATgTTCAgCT TgAgCAgAAg gTCTgAggCA |
| | ACgAAATCTg |
| 2251 | gATCAgCgTA TTTATCAgCA ATAACTAgAA CTTCAgAAgg |
| | CCCAgCAggC |
| 2301 | ATgTCAATAC TACACAgggC TgATgTgTCA TTTTgAACCA |
| | TCATCTTggC |
| 2351 | AgCAgTAACg AACTggTTTC CTggACCAAA TATTTTgTCA |
| | CACTTAggAA |
| 2401 | CAgTTTCTgT TCCgTAAgCC ATAgCAgCTA CTgCCTgggC |
| | gCCTCCTgCT |
| 2451 | AgCACgATAC ACTTAgCACC AACCTTgTgg gCAACgTAgA |
| | TgACTTCTgg |
| 2501 | ggTAAgggTA CCATCCTTCT TAggTggAgA TgCAAAAACA |
| | ATTTCTTTgC |
| 2551 | AACCAgCAAC TTTggCAggA ACACCCAgCA TCAgggAAgT |
| | ggAAggCAgA |
| 2601 | ATTgCggTTC CACCAggAAT ATAgAggCCA ACTTTCTCAA |
| | TAggTCTTgC |
| 2651 | AAAACgAgAg CAgACTACAC CAgggCAAgT CTCAACTTgC |
| | AACgTCTCCg |
| 2701 | TTAgTTgAgC TTCATggAAT TTCCTgACgT TATCTATAgA |
| | gAgATCAATg |
| 2751 | gCTCTCTTAA CgTTATCTgg CAATTgCATA AgTTCCTCTg |
| | ggAAAggAgC |
| 2801 | TTCTAACACA ggTgTCTTCA AAgCgACTCC ATCAAACTTg |
| | gCAgTTAgTT |
| 2851 | CTAAAAgggC TTTgTCACCA TTTTgACgAA CATTgTCgAC |
| | AATTggTTTg |
| 2901 | ACTAATTCCA TAATCTgTTC CgTTTTCTgg ATAggACgAC |
| | gAAgggCATC |
| 2951 | TTCAATTTCT TgTgAggAgg CCTTAgAAAC gTCAATTTTg |
| | CACAATTCAA |
| 3001 | TACgACCTTC AgAAgggACT TCTTTAggTT TggATTCTTC |
| | TTTAggTTgT |
| 3051 | TCCTTggTgT ATCCTggCTT ggCATCTCCT TTCCTTCTAg |
| | TgACCTTTAg |
| 3101 | ggACTTCATA TCCAggTTTC TCTCCACCTC gTCCAACgTC |
| | ACACCgTACT |
| 3151 | TggCACATCT AACTAATgCA AAATAAAATA AgTCAgCACA |
| | TTCCCAggCT |
| 3201 | ATATCTTCCT TggATTTAgC TTCTgCAAgT TCATCAgCTT |
| | CCTCCCTAAT |
| 3251 | TTTAgCgTTC AACAAAACTT CgTCgTCAAA TAACCgTTTg |
| | gTATAAgAAC |
| 3301 | CTTCTggAgC ATTgCTCTTA CgATCCCACA AggTgCTTCC |
| | ATggCTCTAA |
| 3351 | gACCCTTTgA TTggCCAAAA CAggAAgTgC gTTCCAAgTg |
| | ACAgAAACCA |
| 3401 | ACACCTgTTT gTTCAACCAC AAATTTCAAg CAgTCTCCAT |
| | CACAATCCAA |
| 3451 | TTCgATACCC AgCAACTTTT gAgTTCgTCC AgATgTAgCA |
| | CCTTTATACC |
| 3501 | ACAAACCgTg ACgACgAgAT TggTAgACTC CAgTTTgTgT |
| | CCTTATAgCC |
| 3551 | TCCggAATAg ACTTTTTggA CgAgTACACC AggCCCAACg |
| | AgTAATTAgA |
| 3601 | AgAgTCAgCC ACCAAAgTAg TgAATAgACC ATCggggCgg |
| | TCAgTAgTCA |
| 3651 | AAgACgCCAA CAAAATTTCA CTgACAgggA ACTTTTTgAC |
| | ATCTTCAgAA |
| 3701 | AgTTCgTATT CAgTAgTCAA TTgCCgAgCA TCAATAATgg |
| | ggATTATACC |
| 3751 | AgAAgCAACA gTggAAgTCA CATCTACCAA CTTTgCggTC |
| | TCAgAAAAAg |
| 3801 | CATAAACAgT TCTACTACCg CCATTAgTgA AACTTTTCAA |
| | ATCgCCCAgT |
| 3851 | ggAgAAgAAA AAggCACAgC gATACTAgCA TTAgCgggCA |
| | AggATgCAAC |
| 3901 | TTTATCAACC AgggTCCTAT AgATAACCCT AgCgCCTggg |
| | ATCATCCTTT |
| 3951 | ggACAACTCT TTCTgCCAAA TCTAggTCCA AAATCACTTC |
| | ATTgATACCA |
| 4001 | TTATACggAT gACTCAACTT gCACATTAAC TTgAAgCTCA |
| | gTCgATTgAg |
| 4051 | TgAACTTgAT CAggTTgTgC AgCTggTCAg CAgCATAggg |
| | AAACACggCT |
| 4101 | TTTCCTACCA AACTCAAggA ATTATCAAAC TCTgCAACAC |
| | TTgCgTATgC |
| 4151 | AggTAgCAAg ggAAATgTCA TACTTgAAgT CggACAgTgA |
| | gTgTAgTCTT |
| 4201 | gAgAAATTCT gAAgCCgTAT TTTTATTATC AgTgAgTCAg |
| | TCATCAggAg |
| 4251 | ATCCTCTACg CCggACgCAT CgTggCCggC ATCACCggCg |
| | CCACAggTgC |
| 4301 | ggTTgCTggC gCCTATATCg CCgACATCAC CgATggggAA |
| | gATCgggCTC |
| 4351 | gCCACTTCgg gCTCATgAgC gCTTgTTTCg gCgTgggTAT |
| | ggTggCAggC |
| 4401 | CCCgTggCCg ggggACTgTT gggCgCCATC TCCTTgCATg |
| | CACCATTCCT |
| 4451 | TgCggCggCg gTgCTCAACg gCCTCAACCT ACTACTgggC |
| | TgCTTCCTAA |
| 4501 | TgCAggAgTC gCATAAgggA gAgCgTCgAg TATCTATgAT |
| | TggAAgTATg |
| 4551 | ggAATggTgA TACCCgCATT CTTCAgTgTC TTgAggTCTC |
| | CTATCAgATT |
| 4601 | ATgCCCAACT AAAgCAACCg gAggAggAgA TTTCATggTA |
| | AATTTCTCTg |
| 4651 | ACTTTTggTC ATCAgTAgAC TCgAACTgTg AgACTATCTC |
| | ggTTATgACA |
| 4701 | gCAgAAATgT CCTTCTTggA gACAgTAAAT gAAgTCCCAC |
| | CAATAAAgAA |
| 4751 | ATCCTTgTTA TCAggAACAA ACTTCTTgTT TCgAACTTTT |
| | TCggTgCCTT |
| 4801 | gAACTATAAA ATgTAgAgTg gATATgTCgg gTAggAATgg |
| | AgCgggCAAA |
| 4851 | TgCTTACCTT CTggACCTTC AAgAggTATg TAgggTTTgT |
| | AgATACTgAT |
| 4901 | gCCAACTTCA gTgACAACgT TgCTATTTCg TTCAAACCAT |
| | TCCgAATCCA |
| 4951 | gAgAAATCAA AgTTgTTTgT CTACTATTgA TCCAAgCCAg |
| | TgCggTCTTg |
| 5001 | AAACTgACAA TAgTgTgCTC gTgTTTTgAg gTCATCTTTg |
| | TATgAATAAA |
| 5051 | TCTAgTCTTT gATCTAAATA ATCTTgACgA gCCAAggCgA |
| | TAAATACCCA |
| 5101 | AATCTAAAAC TCTTTTAAAA CgTTAAAAgg ACAAgTATgT |
| | CTgCCTgTAT |
| 5151 | TAAACCCCAA ATCAgCTCgT AgTCTgATCC TCATCAACTT |
| | gAggggCACT |
| 5201 | ATCTTgTTTT AgAgAAATTT gCggAgATgC gATATCgAgA |
| | AAAAggTACg |
| 5251 | CTgATTTTAA ACgTgAAATT TATCTCAAgA TCgCggCCgC |
| | gATCTCgAAT |
| 5301 | AATAACTgTT ATTTTTCAgT gTTCCCgATC TgCgTCTATT |
| | TCACAATACC |
| 5351 | AACATgAgTC AgCTTATCgA TgATAAgCTg TCAAACATgA |
| | gAATTAATTC |
| 5401 | gATgATAAgC TgTCAAACAT gAgAAATCTT gAAgACgAAA |
| | gggCCTCgTg |
| 5451 | ATACgCCTAT TTTTATAggT TAATgTCATg ATAATAATgg |
| | TTTCTTAgAC |
| 5501 | gTCAggTggC ACTTTTCggg gAAATgTgCg CggAACCCCT |
| | ATTTgTTTAT |
| 5551 | TTTTCTAAAT ACATTCAAAT ATgTATCCgC TCATgAgACA |
| | ATAACCCTgA |
| 5601 | TAAATgCTTC AATAATATTg AAAAAggAAg AgTATgAgTA |
| | TTCAACATTT |
| 5651 | CCgTgTCgCC CTTATTCCCT TTTTTgCggC ATTTTgCCTT |
| | CCTgTTTTTg |
| 5701 | CTCACCCAgA AACgCTggTg AAAgTAAAAg ATgCTgAAgA |
| | TCAgTTgggT |
| 5751 | gCACgAgTgg gTTACATCgA ACTggATCTC AACAgCggTA |
| | AgATCCTTgA |
| 5801 | gAgTTTTCgC CCCgAAgAAC gTTTTCCAAT gATgAgCACT |
| | TTTAAAgTTC |
| 5851 | TgCTATgTgg CgCggTATTA TCCCgTgTTg ACgCCgggCA |
| | AgAgCAACTC |
| 5901 | ggTCgCCgCA TACACTATTC TCAgAATgAC TTggTTgAgT |
| | ACTCACCAgT |
| 5951 | CACAgAAAAg CATCTTACgg ATggCATgAC AgTAAgAgAA |
| | TTATgCAgTg |
| 6001 | CTgCCATAAC CATgAgTgAT AACACTgCgg CCAACTTACT |
| | TCTgACAACg |
| 6051 | ATCggAggAC CgAAggAgCT AACCgCTTTT TTgCACAACA |
| | TgggggATCA |
| 6101 | TgTAACTCgC CTTgATCgTT gggAACCggA gCTgAATgAA |
| | gCCATACCAA |
| 6151 | ACgACgAgCg TgACACCACg ATgCCTgCAg CAATggCAAC |
| | AACgTTgCgC |
| 6201 | AAACTATTAA CTggCgAACT ACTTACTCTA gCTTCCCggC |
| | AACAATTAAT |
| 6251 | AgACTggATg gAggCggATA AAgTTgCAgg ACCACTTCTg |
| | CgCTCggCCC |
| 6301 | TTCCggCTgg CTggTTTATT gCTgATAAAT CTggAgCCgg |
| | TgAgCgTggg |
| 6351 | TCTCgCggTA TCATTgCAgC ACTggggCCA gATggTAAgC |
| | CCTCCCgTAT |
| 6401 | CgTAgTTATC TACACgACgg ggAgTCAggC AACTATggAT |
| | gAACgAAATA |
| 6451 | gACAgATCgC TgAgATAggT gCCTCACTgA TTAAgCATTg |
| | gTAACTgTCA |
| 6501 | gACCAAgTTT ACTCATATAT ACTTTAgATT gATTTAAATT |
| | gTAAACgTTA |
| 6551 | ATATTTTgTT AAAATTCgCg TTAAATTTTT gTTAAATCAg |
| | CTCATTTTTT |
| 6601 | AACCAATAgg CCgAAATCgg CAAAATCCCT TATAAATCAA |
| | AAgAATAgAC |
| 6651 | CgAgATAggg TTgAgTgTTg TTCCAgTTTg gAACAAgAgT |
| | CCACTATTAA |
| 6701 | AgAACgTggA CTCCAACgTC AAAgggCgAA AAACCgTCTA |
| | TCAgggCgAT |
| 6751 | ggCCCACTAC gTgAACCATC ACCCTAATCA AgTTTTTTgg |
| | ggTCgAggTg |
| 6801 | CCgTAAAgCA CTAAATCggA ACCCTAAAgg gAgCCCCCgA |
| | TTTAgAgCTT |
| 6851 | gACggggAAA gCCggCgAAC gTggCgAgAA AggAAgggAA |
| | gAAAgCgAAA |
| 6901 | ggAgCgggCg CTAgggCgCT ggCAAgTgTA gCggTCACgC |
| | TgCgCgTAAC |
| 6951 | CACCACACCC gCCgCgCTTA ATgCgCCgCT ACAgggCgCg |
| | TAAAAggATC |
| 7001 | TAggTgAAgA TCCTTTTTgA TAATCTCATg ACCAAAATCC |
| | CTTAACgTgA |
| 7051 | gTTTTCgTTC CACTgAgCgT CAgACCCCgT AgAAAAgATC |
| | AAAggATCTT |
| 7101 | CTTgAgATCC TTTTTTTCTg CgCgTAATCT gCTgCTTgCA |
| | AACAAAAAAA |
| 7151 | CCACCgCTAC CAgCggTggT TTgTTTgCCg gATCAAgAgC |
| | TACCAACTCT |
| 7201 | TTTTCCgAAg gTAACTggCT TCAgCAgAgC gCAgATACCA |
| | AATACTgTCC |
| 7251 | TTCTAgTgTA gCCgTAgTTA ggCCACCACT TCAAgAACTC |
| | TgTAgCACCg |
| 7301 | CCTACATACC TCgCTCTgCT AATCCTgTTA CCAgTggCTg |
| | CTgCCAgTgg |
| 7351 | CgATAAgTCg TgTCTTACCg ggTTggACTC AAgACgATAg |
| | TTACCggATA |
| 7401 | AggCgCAgCg gTCgggCTgA ACggggggTT CgTgCACACA |
| | gCCCAgCTTg |
| 7451 | gAgCgAACgA CCTACACCgA ACTgAgATAC CTACAgCgTg |
| | AgCATTgAgA |
| 7501 | AAgCgCCACg CTTCCCgAAg ggAgAAAggC ggACAggTAT |
| | CCggTAAgCg |
| 7551 | gCAgggTCgg AACAggAgAg CgCACgAggg AgCTTCCAgg |
| | gggAAACgCC |
| 7601 | TggTATCTTT ATAgTCCTgT CgggTTTCgC CACCTCTgAC |
| | TTgAgCgTCg |
| 7651 | ATTTTTgTgA TgCTCgTCAg gggggCggAg CCTATggAAA |
| | AACgCCAgCA |
| 7701 | ACgCggCCTT TTTACggTTC CTggCCTTTT gCTggCCTTT |
| | TgCTCACATg |
| 7751 | TTCTTTCCTg CgTTATCCCC TgATTCTgTg gATAACCgTA |
| | TTACCgCCTT |
| 7801 | TgAgTgAgCT gATACCgCTC gCCgCAgCCg AACgACCgAg |
| | CgCAgCgAgT |
| 7851 | CAgTgAgCgA ggAAgCggAA gAgCgCCTgA TgCggTATTT |
| | TCTCCTTACg |
| 7901 | CATCTgTgCg gTATTTCACA CCgCATATgg TgCACTCTCA |
| | gTACAATCTg |
| 7951 | CTCTgATgCC gCATAgTTAA gCCAgTATAC ACTCCgCTAT |
| | CgCTACgTgA |
| 8001 | CTgggTCATg gCTgCgCCCC gACACCCgCC AACACCCgCT |
| | gACgCgCCCT |
| 8051 | gACgggCTTg TCTgCTCCCg gCATCCgCTT ACAgACAAgC |
| | TgTgACCgTC |
| 8101 | TCCgggAgCT gCATgTgTCA gAggTTTTCA CCgTCATCAC |
| | CgAAACgCgC |
| 8151 | gAggCAg |

TABLE 24

pHIL-D2(MFαPrePro::EPI-HNE-3) 8584 b.p.
DNA has SEQ ID NO. 071; Encoded polypeptide has
SEQ ID NO. 072. DNA is circular and double
stranded, only one strand is shown. Translation
of the protein to be expressed is shown.

| | |
|---|---|
| | 1 2 3 4 5 |
| | 1234567890 1234567890 1234567890 1234567890 |
| | 1234567890 |
| 1 | AgATCgCggC CgCgATCTAA CATCCAAAgA CgAAAggTTg |
| | AATgAAACCT |
| 51 | TTTTgCCATC CgACATCCAC AggTCCATTC TCACACATAA |
| | gTgCCAAACg |
| 101 | CAACAggAgg ggATACACTA gCAgCAgACC gTTgCAAACg |
| | CAggACCTCC |
| 151 | ACTCCTCTTC TCCTCAACAC CCACTTTTgC CATCgAAAAA |
| | CCAgCCCAgT |
| 201 | TATTgggCTT gATTggAgCT CgCTCATTCC AATTCCTTCT |
| | ATTAggCTAC |
| 251 | TAACACCATg ACTTTATTAg CCTgTCTATC CTggCCCCCC |
| | TggCgAggTC |
| 301 | ATgTTTgTTT ATTTCCgAAT gCAACAAgCT CCgCATTACA |
| | CCCgAACATC |
| 351 | ACTCCAgATg AgggCTTTCT gAgTgTgggg TCAAATAgTT |
| | TCATgTTCCC |
| 401 | AAATggCCCA AAACTgACAg TTTAAACgCT gTCTTggAAC |
| | CTAATATgAC |
| 451 | AAAAgCgTgA TCTCATCCAA gATgAACTAA gTTTggTTCg |
| | TTgAAATgCT |
| 501 | AACggCCAgT TggTCAAAAA gAAACTTCCA AAAgTCgCCA |
| | TACCgTTTgT |
| 551 | CTTgTTTggT ATTgATTgAC gAATgCTCAA AAATAATCTC |
| | ATTAATgCTT |
| 601 | AgCgCAgTCT CTCTATCgCT TCTgAACCCg gTggCACCTg |
| | TgCCgAAACg |
| 651 | CAAATggggA AACAACCCgC TTTTTggATg ATTATgCATT |
| | gTCCTCCACA |
| 701 | TTgTATgCTT CCAAgATTCT ggTgggAATA CTgCTgATAg |
| | CCTAACgTTC |
| 751 | ATgATCAAAA TTTAACTgTT CTAACCCCTA CTTgACAggC |
| | AATATATAAA |
| 801 | CAgAAggAAg CTgCCCTgTC TTAAACCTTT TTTTTTATCA |
| | TCATTATTAg |
| 851 | CTTACTTTCA TAATTgCgAC TggTTCCAAT TgACAAgCTT |
| | TTgATTTTAA |
| 901 | CgACTTTTAA CgACAACTTg AgAAgATCAA AAAACAACTA |
| | ATTATTCgAA |

```
! BstBI
      ACg
! M R F P S I F T A V L F A 13
      ATg AgA TTC CCA TCT ATC TTC ACT gCT gTT TTg TTC
      gCT
! | BsαBI |
!
! A S S A L A A P V N T T T E 27
  gCT TCC TCT gCT TTg gCT gCT CCA gTT AAC ACC ACT
  ACT gAA
! BpmI HpαI BbsI
!
! D E T A Q I P A E A V I G Y 41
  gAC gAg ACT gCT CAA ATT CCT gCT gAg gCT gTC ATC
  ggT TAC
!BbsI
!
! S D L E G D F D V A V L P F 55
  TCT gAC TTg gAA ggT gAC TTC gAC gTC gCT gTT TTg
  CCA TTC
! AαtII
!
! S N S T N N G L L F I N T T 69
  TCT AAC TCT ACT AAC AAC ggT TTg TTg TTC ATC AAC
  ACT ACC
!
! I A S I A A K E E G V S L D 83
  ATC gCT TCT ATC gCT gCT AAg gAg gAA ggT gTT TCC
  TTg gAC
!
! K R A A C N L P 91
  AAg AgA gCT gCT TgT AAC TTg CCA
          |------Site of cleavage
!
! I V R G P C I A F F P R W A 105
  ATC gTC AgA ggT CCA TgC ATT gCT TTC TTC CCA AgA
  Tgg gCT
! NsiI
!
! F D A V K G K C V L F P Y G 119
  TTC gAC gCT gTT AAg ggT AAg TgC gTC TTg TTC CCA
  TAC ggT
! | PflMI
!
! G C Q G N G N K F Y S E K E 133
  ggT TgT CAA ggT AAC ggT AAC AAg TTC TAC TCT gAg
  AAg gAg
!PflMI
!
! C R E Y C G V P . . 141
  TgT AgA gAg TAC TgT ggT gTT CCA TAg TAA
  gAATTCgCCT
! EcoRI
                                                    TAgACATg
```

| | |
|---|---|
| 1401 | ACTgTTCCTC AgTTCAAgTT gggCATTACg AgAAgACCgg |
| | TCTTgCTAgA |
| 1451 | TTCTAATCAA gAggATgTCA gAATgCCATT TgCCTgAgAg |
| | ATgCAggCTT |
| 1501 | CATTTTTgAT ACTTTTTTAT TTgTAACCTA TATAgTATAg |
| | gATTTTTTTT |
| 1551 | gTCATTTTgT TTCTTCTCgT ACgAgCTTgC TCCTgATCAg |
| | CCTATCTCgC |
| 1601 | AgCTgATgAA TATCTTgTgg TAggggTTTg ggAAAATCAT |
| | TCgAgTTTgA |
| 1651 | TgTTTTTCTT ggTATTTCCC ACTCCTCTTC AgAgTACAgA |
| | AgATTAAgTg |
| 1701 | AgAAgTTCgT TTgTgCAAgC TTATCgATAA gCTTTAATgC |
| | ggTAgTTTAT |
| 1751 | CACAgTTAAA TTgCTAACgC AgTCAggCAC CgTgTATgAA |
| | ATCTAACAAT |
| 1801 | gCgCTCATCg TCATCCTCgg CACCgTCACC CTggATgCTg |
| | TAggCATAgg |
| 1851 | CTTggTTATg CCggTACTgC CgggCCTCTT gCgggATATC |
| | gTCCATTCCg |
| 1901 | ACAgCATCgC CAgTCACTAT ggCgTgCTgC TAgCgCTATA |
| | TgCgTTgATg |
| 1951 | CAATTTCTAT gCgCACCCgT TCTCggAgCA CTgTCCgACC |
| | gCTTTggCCg |
| 2001 | CCgCCCAgTC CTgCTCgCTT CgCTACTTgg AgCCACTATC |
| | gACTACgCgA |
| 2051 | TCATggCgAC CACACCCgTC CTgTggATCT ATCgAATCTA |
| | AATgTAAgTT |
| 2101 | AAAATCTCTA AATAATTAAA TAAgTCCCAg TTTCTCCATA |
| | CgAACCTTAA |
| 2151 | CAgCATTgCg gTgAgCATCT AgACCTTCAA CAgCAgCCAg |
| | ATCCATCACT |
| 2201 | gCTTggCCAA TATgTTTCAg TCCCTCAggA gTTACgTCTT |
| | gTgAAgTgAT |
| 2251 | gAACTTCTgg AAggTTgCAg TgTTAACTCC gCTgTATTgA |
| | CgggCATATC |
| 2301 | CgTACgTTgg CAAAgTgTgg TTggTACCgg AggAgTAATC |
| | TCCACAACTC |
| 2351 | TCTggAgAgT AggCACCAAC AAACACAgAT CCAgCgTgTT |
| | gTACTTgATC |
| 2401 | AACATAAgAA gAAgCATTCT CgATTTgCAg gATCAAgTgT |
| | TCAggAgCgT |
| 2451 | ACTgATTggA CATTTCCAAA gCCTgCTCgT AggTTgCAAC |
| | CgATAgggTT |
| 2501 | gTAgAgTgTg CAATACACTT gCgTACAATT TCAACCCTTg |
| | gCAACTgCAC |
| 2551 | AgCTTggTTg TgAACAgCAT CTTCAATTCT ggCAAgCTCC |
| | TTgTCTgTCA |
| 2601 | TATCgACAgC CAACAgAATC ACCTgggAAT CAATACCATg |
| | TTCAgCTTgA |
| 2651 | gCAgAAggTC TgAggCAACg AAATCTggAT CAgCgTATTT |
| | ATCAgCAATA |
| 2701 | ACTAgAACTT CAgAAggCCC AgCAggCATg TCAATACTAC |
| | ACAgggCTgA |
| 2751 | TgTgTCATTT TgAACCATCA TCTTggCAgC AgTAACgAAC |
| | TggTTTCCTg |
| 2801 | gACCAAATAT TTTgTCACAC TTAggAACAg TTTCTgTTCC |
| | gTAAgCCATA |
| 2851 | gCAgCTACTg CCTgggCgCC TCCTgCTAgC ACgATACACT |
| | TAgCACCAAC |
| 2901 | CTTgTgggCA ACgTAgATgA CTTCTggggT AAgggTACCA |
| | TCCTTCTTAg |
| 2951 | gTggAgATgC AAAAACAATT TCTTTgCAAC CAgCAACTTT |
| | ggCAggAACA |
| 3001 | CCCAgCATCA gggAAgTggA AggCAgAATT gCggTTCCAC |
| | CAggAATATA |
| 3051 | gAggCCAACT TTCTCAATAg gTCTTgCAAA ACgAgAgCAg |
| | ACTACACCAg |
| 3101 | ggCAAgTCTC AACTTgCAAC gTCTCCgTTA gTTgAgCTTC |
| | ATggAATTTC |
| 3151 | CTgACgTTAT CTATAgAgAg ATCAATggCT CTCTTAACgT |
| | TATCTggCAA |
| 3201 | TTgCATAAgT TCCTCTgggA AAggAgCTTC TAACACAggT |
| | gTCTTCAAAg |
| 3251 | CgACTCCATC AAACTTggCA gTTAgTTCTA AAAgggCTTT |
| | gTCACCATTT |
| 3301 | TgACgAACAT TgTCgACAAT TggTTTgACT AATTCCATAA |
| | TCTgTTCCgT |
| 3351 | TTTCTggATA ggACgACgAA gggCATCTTC AATTTCTTgT |
| | gAggAggCCT |
| 3401 | TAgAAACgTC AATTTTgCAC AATTCAATAC gACCTTCAgA |
| | AgggACTTCT |
| 3451 | TTAggTTTgg ATTCTTCTTT AggTTgTTCC TTggTgTATC |
| | CTggCTTggC |
| 3501 | ATCTCCTTTC CTTCTAgTgA CCTTTAgggA CTTCATATCC |
| | AggTTTCTCT |
| 3551 | CCACCTCgTC CAACgTCACA CCgTACTTgg CACATCTAAC |
| | TAATgCAAAA |
| 3601 | TAAAATAAgT CAgCACATTC CCAggCTATA TCTTCCTTgg |
| | ATTTAgCTTC |
| 3651 | TgCAAgTTCA TCAgCTTCCT CCCTAATTTT AgCgTTCAAC |
| | AAAACTTCgT |
| 3701 | CgTCAAATAA CCgTTTggTA TAAgAACCTT CTggAgCATT |
| | gCTCTTACgA |
| 3751 | TCCCACAAgg TgCTTCCATg gCTCTAAgAC CCTTTgATTg |
| | gCCAAAACAg |
| 3801 | gAAgTgCgTT CCAAgTgACA gAAACCAACA CCTgTTTgTT |
| | CAACCACAAA |
| 3851 | TTTCAAgCAg TCTCCATCAC AATCCAATTC gATACCCAgC |
| | AACTTTTgAg |
| 3901 | TTCgTCCAgA TgTAgCACCT TTATACCACA AACCgTgACg |
| | ACgAgATTgg |
| 3951 | TAgACTCCAg TTTgTgTCCT TATAgCCTCC ggAATAgACT |
| | TTTTggACgA |
| 4001 | gTACACCAgg CCCAACgAgT AATTAgAAgA gTCAgCCACC |
| | AAAgTAgTgA |
| 4051 | ATAgACCATC ggggCggTCA gTAgTCAAAg ACgCCAACAA |
| | AATTTCACTg |
| 4101 | ACAgggAACT TTTTgACATC TTCAgAAAgT TCgTATTCAg |
| | TAgTCAATTg |
| 4151 | CCgAgCATCA ATAATggggA TTATACCAgA AgCAACAgTg |
| | gAAgTCACAT |
| 4201 | CTACCAACTT TgCggTCTCA gAAAAAgCAT AAACAgTTCT |
| | ACTACCgCCA |
| 4251 | TTAgTgAAAC TTTTCAAATC gCCCAgTggA gAAgAAAAAg |
| | gCACAgCgAT |
| 4301 | ACTAgCATTA gCgggCAAgg ATgCAACTTT ATCAACCAgg |
| | gTCCTATAgA |
| 4351 | TAACCCTAgC gCCTgggATC ATCCTTTggA CAACTCTTTC |
| | TgCCAAATCT |
| 4401 | AggTCCAAAA TCACTTCATT gATACCATTA TACggATgAC |
| | TCAACTTgCA |
| 4451 | CATTAACTTg AAgCTCAgTC gATTgAgTgA ACTTgATCAg |
| | gTTgTgCAgC |
| 4501 | TggTCAgCAg CATAgggAAA CACggCTTTT CCTACCAAAC |
| | TCAAggAATT |
| 4551 | ATCAAACTCT gCAACACTTg CgTATgCAgg TAgCAAgggA |
| | AATgTCATAC |
| 4601 | TTgAAgTCgg ACAgTgAgTg TAgTCTTgAg AAATTCTgAA |
| | gCCgTATTTT |
| 4651 | TATTATCAgT gAgTCAgTCA TCAggAgATC CTCTACgCCg |
| | gACgCATCgT |
| 4701 | ggCCggCATC ACCggCgCCA CAggTgCggT TgCTggCgCC |
| | TATATCgCCg |
| 4751 | ACATCACCgA TggggAAgAT CgggCTCgCC ACTTCgggCT |
| | CATgAgCgCT |
| 4801 | TgTTTCggCg TgggTATggT ggCAggCCCC gTggCCgggg |
| | gACTgTTggg |
| 4851 | CgCCATCTCC TTgCATgCAC CATTCCTTgC ggCggCggTg |
| | CTCAACggCC |
| 4901 | TCAACCTACT ACTgggCTgC TTCCTAATgC AggAgTCgCA |
| | TAAgggAgAg |
| 4951 | CgTCgAgTAT CTATgATTgg AAgTATgggA ATggTgATAC |
| | CCgCATTCTT |
| 5001 | CAgTgTCTTg AggTCTCCTA TCAgATTATg CCCAACTAAA |
| | gCAACCggAg |
| 5051 | gAggAgATTT CATggTAAAT TTCTCTgACT TTTggTCATC |
| | AgTAgACTCg |
| 5101 | AACTgTgAgA CTATCTCggT TATgACAgCA gAAATgTCCT |
| | TCTTggAgAC |
| 5151 | AgTAAATgAA gTCCCACCAA TAAAgAAATC CTTgTTATCA |
| | ggAACAAACT |
| 5201 | TCTTgTTTCg AACTTTTTCg gTgCCTTgAA CTATAAAATg |
| | TAgAgTggAT |
| | BstBI |
| 5251 | ATgTCgggTA ggAATggAgC gggCAAATgC TTACCTTCTg |
| | gACCTTCAAg |
| 5301 | AggTATgTAg ggTTTgTAgA TACTgATgCC AACTTCAgTg |
| | ACAACgTTgC |
| 5351 | TATTTCgTTC AAACCATTCC gAATCCAgAg AAATCAAAgT |
| | TgTTTgTCTA |
| 5401 | CTATTgATCC AAgCCAgTgC ggTCTTgAAA CTgACAATAg |
| | TgTgCTCgTg |
| 5451 | TTTTgAggTC ATCTTTgTAT gAATAAATCT AgTCTTTgAT |
| | CTAAATAATC |
| 5501 | TTgACgAgCC AAggCgATAA ATACCCAAAT CTAAAACTCT |
| | TTTAAAACgT |
| 5551 | TAAAAggACA AgTATgTCTg CCTgTATTAA ACCCCAAATC |
| | AgCTCgTAgT |
| 5601 | CTgATCCTCA TCAACTTgAg gggCACTATC TTgTTTTAgA |
| | gAAATTTgCg |
| 5651 | gAgATgCgAT ATCgAgAAAA AggTACgCTg ATTTTAAACg |
| | TgAAATTTAT |
| 5701 | CTCAAgATCg CggCCgCgAT CTCgAATAAT AACTgTTATT |
| | TTTCAgTgTT |
| 5751 | CCCgATCTgC gTCTATTTCA CAATACCAAC ATgAgTCAgC |
| | TTATCgATgA |
| 5801 | TAAgCTgTCA AACATgAgAA TTAATTCgAT gATAAgCTgT |
| | CAAACATgAg |
| 5851 | AAATCTTgAA gACgAAAggg CCTCgTgATA CgCCTATTTT |
| | TATAggTTAA |
| 5901 | TgTCATgATA ATAATggTTT CTTAgACgTC AggTggCACT |
| | TTTCggggAA |
| | AαtII |
| 5951 | ATgTgCgCgg AACCCCTATT TgTTTATTTT TCTAAATACA |
| | TTCAAATATg |
| 6001 | TATCCgCTCA TgAgACAATA ACCCTgATAA ATgCTTCAAT |
| | AATATTgAAA |
| 6051 | AAggAAgAgT ATgAgTATTC AACATTTCCg TgTCgCCCTT |
| | ATTCCCTTTT |
| 6101 | TTgCggCATT TTgCCTTCCT gTTTTTgCTC ACCCAgAAAC |
| | gCTggTgAAA |
| 6151 | gTAAAAgATg CTgAAgATCA gTTgggTgCA CgAgTgggTT |
| | ACATCgAACT |
| 6201 | ggATCTCAAC AgCggTAAgA TCCTTgAgAg TTTTCgCCCC |
| | gAAgAACgTT |
| 6251 | TTCCAATgAT gAgCACTTTT AAAgTTCTgC TATgTggCgC |
| | ggTATTATCC |
| 6301 | CgTgTTgACg CCgggCAAgA gCAACTCggT CgCCgCATAC |
| | ACTATTCTCA |
| 6351 | gAATgACTTg gTTgAgTACT CACCAgTCAC AgAAAAgCAT |
| | CTTACggATg |
| 6401 | gCATgACAgT AAgAgAATTA TgCAgTgCTg CCATAACCAT |
| | gAgTgATAAC |
| 6451 | ACTgCggCCA ACTTACTTCT gACAACgATC ggAggACCgA |
| | AggAgCTAAC |
| 6501 | CgCTTTTTTg CACAACATgg gggATCATgT AACTCgCCTT |
| | gATCgTTggg |
| 6551 | AACCggAgCT gAATgAAgCC ATACCAAACg ACgAgCgTgA |
| | CACCACgATg |
| 6601 | CCTgCAgCAA TggCAACAAC gTTgCgCAAA CTATTAACTg |
| | gCgAACTACT |
| 6651 | TACTCTAgCT TCCCggCAAC AATTAATAgA CTggATggAg |
| | gCggATAAAg |
| 6701 | TTgCAggACC ACTTCTgCgC TCggCCCTTC CggCTggCTg |
| | gTTTATTgCT |
| 6751 | gATAAATCTg gAgCCggTgA gCgTgggTCT CgCggTATCA |
| | TTgCAgCACT |
| 6801 | ggggCCAgAT ggTAAgCCCT CCCgTATCgT AgTTATCTAC |
| | ACgACggggA |
| 6851 | gTCAggCAAC TATggATgAA CgAAATAgAC AgATCgCTgA |
| | gATAggTgCC |
| 6901 | TCACTgATTA AgCATTggTA ACTgTCAgAC CAAgTTTACT |
| | CATATATACT |
| 6951 | TTAgATTgAT TTAAATTgTA AACgTTAATA TTTTgTTAAA |
| | ATTCgCgTTA |
| 7001 | AATTTTTgTT AAATCAgCTC ATTTTTTAAC CAATAggCCg |
| | AAATCggCAA |
| 7051 | AATCCCTTAT AAATCAAAAg AATAgACCgA gATAgggTTg |
| | AgTgTTgTTC |
| 7101 | CAgTTTggAA CAAgAgTCCA CTATTAAAgA ACgTggACTC |
| | CAACgTCAAA |
| 7151 | gggCgAAAAA CCgTCTATCA gggCgATggC CCACTACgTg |
| | AACCATCACC |
| 7201 | CTAATCAAgT TTTTTggggT CgAggTgCCg TAAAgCACTA |
| | AATCggAACC |
| 7251 | CTAAAgggAg CCCCCgATTT AgAgCTTgAC ggggAAAgCC |
| | ggCgAACgTg |
| 7301 | gCgAgAAAgg AAgggAAgAA AgCgAAAggA gCgggCgCTA |
| | gggCgCTggC |
| 7351 | AAgTgTAgCg gTCACgCTgC gCgTAACCAC CACACCCgCC |
| | gCgCTTAATg |
| 7401 | CgCCgCTACA gggCgCgTAA AAggATCTAg gTgAAgATCC |
| | TTTTTgATAA |
| 7451 | TCTCATgACC AAAATCCCTT AACgTgAgTT TTCgTTCCAC |
| | TgAgCgTCAg |
| 7501 | ACCCCgTAgA AAAgATCAAA ggATCTTCTT gAgATCCTTT |
| | TTTTCTgCgC |
| 7551 | gTAATCTgCT gCTTgCAAAC AAAAAAACCA CCgCTACCAg |
| | CggTggTTTg |
| 7601 | TTTgCCggAT CAAgAgCTAC CAACTCTTTT TCCgAAggTA |
| | ACTggCTTCA |
| 7651 | gCAgAgCgCA gATACCAAAT ACTgTCCTTC TAgTgTAgCC |
| | gTAgTTAggC |
| 7701 | CACCACTTCA AgAACTCTgT AgCACCgCCT ACATACCTCg |
| | CTCTgCTAAT |
| 7751 | CCTgTTACCA gTggCTgCTg CCAgTggCgA TAAgTCgTgT |
| | CTTACCgggT |
| 7801 | TggACTCAAg ACgATAgTTA CCggATAAgg CgCAgCggTC |
| | gggCTgAACg |
| 7851 | gggggTTCgT gCACACAgCC CAgCTTggAg CgAACgACCT |
| | ACACCgAACT |
| 7901 | gAgATACCTA CAgCgTgAgC ATTgAgAAAg CgCCACgCTT |
| | CCCgAAgggA |
| 7951 | gAAAggCggA CAggTATCCg gTAAgCggCA gggTCggAAC |
| | AggAgAgCgC |
| 8001 | ACgAgggAgC TTCCAggggg AAACgCCTgg TATCTTTATA |
| | gTCCTgTCgg |
| 8051 | gTTTCgCCAC CTCTgACTTg AgCgTCgATT TTTgTgATgC |
| | TCgTCAgggg |
| 8101 | ggCggAgCCT ATggAAAAAC gCCAgCAACg CggCCTTTTT |
| | ACggTTCCTg |
| 8151 | gCCTTTTgCT ggCCTTTTgC TCACATgTTC TTTCCTgCgT |
| | TATCCCCTgA |
| 8201 | TTCTgTggAT AACCgTATTA CCgCCTTTgA gTgAgCTgAT |
| | ACCgCTCgCC |
| 8251 | gCAgCCgAAC gACCgAgCgC AgCgAgTCAg TgAgCgAggA |
| | AgCggAAgAg |
| 8301 | CgCCTgATgC ggTATTTTCT CCTTACgCAT CTgTgCggTA |
| | TTTCACACCg |
| 8351 | CATATggTgC ACTCTCAgTA CAATCTgCTC TgATgCCgCA |
| | TAgTTAAgCC |
| 8401 | AgTATACACT CCgCTATCgC TACgTgACTg ggTCATggCT |
| | gCgCCCCgAC |
| 8451 | ACCCgCCAAC ACCCgCTgAC gCgCCCTgAC gggCTTgTCT |
| | gCTCCCggCA |
| 8501 | TCCgCTTACA gACAAgCTgT gACCgTCTCC gggAgCTgCA |
| | TgTgTCAgAg |
| 8551 | gTTTTCACCg TCATCACCgA AACgCgCgAg gCAg |

Restriction map of pHIL-D2(MFαPrePro::EPI-HNE-3)
Non-cutters

| | | | | |
|---|---|---|---|---|
| AflII | ApαI | AscI | AvαI | AvrII |
| BαmHI | BglII | BssHII | BstEII | MluI |
| NruI | PαcI | PmlI | RsrII | SαcII |
| SfiI | SnαBI | SpeI | XhoI | XmαI |

Cutters, 3 or fewer sites

| | | | |
|---|---|---|---|
| AαtII | 2 1098 5925 | ApαLI | 3 6176 7859 8357 |
| AflIII | 1 8173 | AseI | 3 591 5820 6672 |
| AgeI | 1 1436 | BglI | 3 284 2717 6724 |
| AlwNI | 3 2828 2852 | BsαAI | 2 7185 8421 |
| | 7759 | | |
| BsgI | 2 2545 4494 | Ecl36I | 1 216 |
| BsiWI | 2 1568 2301 | Eco47III | 2 1932 4795 |
| BspDI | 2 1723 5793 | EcoNI | 3 3433 4923 5293 |
| BspEI | 1 3978 | EcoRI | 1 1383 |
| BspMI | 1 4576 | EcoRV | 2 1885 5658 |
| Bst1107I | 1 8402 | Esp3I(BsαI) | 2 3120 8524 |
| BstBI | 2 945 5207 | EspI | 1 597 |
| (AsuII) | | (Bpu1102I) | |
| BstXI | 3 711 2765 | FspI | 2 1960 6623 |
| | 2896 | | |
| Bsu36I | 1 2223 | HindIII | 3 885 1717 1729 |
| DrαIII | 2 3754 7182 | HpαI | 2 1017 2272 |
| EαgI | 3 7 5711 8591 | KpnI | 2 2323 2934 |
| Eαm1105I | 2 5077 6843 | MscI | 2 2204 3789 |
| NcoI | 1 3766 | | |
| NdeI | 1 8351 | | |
| NgoMI | 2 4702 7288 | | |
| NheI | 2 1929 2875 | | |
| NotI | 3 6 5710 8590 | | |
| NsiI | 2 684 1241 | | |
| PflMI | 2 196 1302 | | |
| PmeI | 1 420 | | |
| PpuMI | 2 142 4339 | | |
| PstI | 1 6602 | | |
| PvuI | 1 6476 | | |
| PvuII | 2 1600 4497 | | |
| SαcI | 1 216 | | |
| SαlI | 1 3312 | | |
| ScαI | 2 1360 6365 | | |
| SphI | 1 4863 | | |
| SspI | 3 2806 6041 | | |
| | 6977 | | |
| StuI | 1 3395 | | |
| Tth111I | 1 8426 | | |
| XbαI | 1 2168 | | |
| XcmI | 1 711 | | |

TABLE 25

BstBI-AatII-EcoRI cassette for expression of
EPI-HNE-4 DNA has SEQ ID NO. 073; amino-acid
sequence has SEQ ID NO. 074

```
! M R F P S I F T
  5′TTCgAA ACg ATg AgA TTC CCA TCT ATC TTC ACT
      BstBI | BsαBI |
! A V L F A 13
          gCT gTT TTg TTC gCT
!
! A S S A L A A P V N T T T E 27
  gCT TCC TCT gCT TTg gCT gCT CCA gTT AAC ACC ACT
  ACT gAA
! BpmI HpαI BbsI
!
! D E T A Q I P A E A V I G Y 41
  gAC gAg ACT gCT CAA ATT CCT gCT gAg gCT gTC ATC
  ggT TAC
!BbsI
!
! S D L E G D F D V A V L P F 55
  TCT gAC TTg gAA ggT gAC TTC gAC gTC gCT gTT TTg
  CCA TTC
! AαtII
!
! S N S T N N G L L F I N T T 69
  TCT AAC TCT ACT AAC AAC ggT TTg TTg TTC ATC AAC
  ACT ACC
!
! I A S I A A K E E G V S L D 83
  ATC gCT TCT ATC gCT gCT AAg gAg gAA ggT gTT TCC
  TTg gAC
!
! K R E A C N L P 91
  AAg AgA gAg gCT TgT AAC TTg CCA
!
! I V R G P C I A F F P R W A 105
  ATC gTC AgA ggT CCA TgC ATT gCT TTC TTC CCA AgA
  Tgg gCT
! NsiI
!
! F D A V K G K C V L F P Y G 119
  TTC gAC gCT gTT AAg ggT AAg TgC gTC TTg TTC CCA
  TAC ggT
! | PflMI
!
! G C Q G N G N K F Y S E K E 133
  ggT TgT CAA ggT AAC ggT AAC AAg TTC TAC TCT gAg
  AAg gAg
!PflMI
!
! C R E Y C G V P . . 141
  TgT AgA gAg TAC TgT ggT gTT CCA TAg TAA gAATTC
! EcoRI
```

The DNA is a linear fragment that is double stranded in vivo, only one strand is shown.
The amino acid sequence is that of a disulfide-containing protein that is processed in vivo.

TABLE 253

pD2pick(MFαPrePro::EPI-HNE-3), 8590 bp, CIRCULAR
dsDNA, one strand shown. pD2pick(MFαPrePro::EPI-
HNE-3) DNA has SEQ ID NO. 075 Encoded protein has
SEQ ID NO. 076

| | |
|---|---|
| | 1 2 3 4 5 |
| | 1234567890 1234567890 1234567890 1234567890 |
| | 1234567890 |
| 1 | AgATCgCggC CgCgATCTAA CATCCAAAgA CgAAAggTTg |
| | AATgAAACCT |
| 51 | TTTTgCCATC CgACATCCAC AggTCCATTC TCACACATAA |
| | gTgCCAAACg |
| 101 | CAACAggAgg ggATACACTA gCAgCAgACC gTTgCAAACg |
| | CAggACCTCC |
| 151 | ACTCCTCTTC TCCTCAACAC CCACTTTTgC CATCgAAAAA |
| | CCAgCCCAgT |
| 201 | TATTgggCTT gATTggAgCT CgCTCATTCC AATTCCTTCT |
| | ATTAggCTAC |
| | SαcI |
| 251 | TAACACCATg ACTTTATTAg CCTgTCTATC CTggCCCCCC |
| | TggCgAggTC |
| 301 | ATgTTTgTTT ATTTCCgAAT gCAACAAgCT CCgCATTACA |
| | CCCgAACATC |
| 351 | ACTCCAgATg AgggCTTTCT gAgTgTgggg TCAAATAgTT |
| | TCATgTTCCC |
| 401 | AAATggCCCA AAACTgACAg TTTAAACgCT gTCTTggAAC |
| | CTAATATgAC |
| | PmeI |
| 451 | AAAAgCgTgA TCTCATCCAA gATgAACTAA gTTTggTTCg |
| | TTgAAATgCT |
| 501 | AACggCCAgT TggTCAAAAA gAAACTTCCA AAAgTCgCCA |
| | TACCgTTTgT |
| 551 | CTTgTTTggT ATTgATTgAC gAATgCTCAA AAATAATCTC |
| | ATTAATgCTTAgC |
| | EspI |
| 604 | gCAgTCT CTCTATCgCT TCTgAACCCg gTggCACCTg |
| | TgCCgAAACg |
| 651 | CAAATggggA AACAACCCgC TTTTTggATg ATTATgCATT |
| | gTCCTCCACA |
| 701 | TTgTATgCTT CCAAgATTCT ggTgggAATA CTgCTgATAg |
| | CCTAACgTTC |
| | XcmI |
| 751 | ATgATCAAAA TTTAACTgTT CTAACCCCTA CTTgACAggC |
| | AATATATAAA |
| 801 | CAgAAggAAg CTgCCCTgTC TTAAACCTTT TTTTTTATCA |
| | TCATTATTAg |
| 851 | CTTACTTTCA TAATTgCgAC TggTTCCAAT TgACAAgCTT |
| | TTgATTTTAA |
| 901 | CgACTTTTAA CgACAACTTg AgAAgATCAA AAAACAACTA |
| | ATTATTCgAA |
| ! | BstBI |
| 951 | ACg |
| ! | |
| ! | M R F P S I F T A V L F A |
| 954 | ATg AgA TTC CCA TCT ATC TTC ACT gCT gTT TTg |
| | TTC gCT |
| ! | |
| ! | A S S A L A A P V N T T T |
| 993 | gCT TCC TCT gCT TTg gCT gCT CCA gTT AAC ACC |
| | ACT ACT |
| ! | |
| ! | E D E T A Q I P A E A V I |
| 1032 | gAA gAC gAg ACT gCT CAA ATT CCT gCT gAg gCT |
| | gTC ATC |
| ! | |
| ! | G Y S D L E G D F D V A V |
| 1071 | ggT TAC TCT gAC TTg gAA ggT gAC TTC gAC gTC |
| | gCT gTT |
| | AαtII |
| ! | |
| ! | L P F S N S T N N G L L F |
| 1110 | TTg CCA TTC TCT AAC TCT ACT AAC AAC ggT TTg |
| | TTg TTC |
| ! | |
| ! | I N T T I A S I A A K E E |
| 1149 | ATC AAC ACT ACC ATC gCT TCT ATC gCT gCT AAg |
| | gAg gAA |
| ! | |
| ! | G V S L D K R A A C N L P |
| 1188 | ggT gTT TCC TTg gAC AAg AgA gCT gCT TgT AAC |
| | TTg CCA |
| ! | |
| ! | I V R G P C I A F F P R W |
| 1227 | ATC gTC AgA ggT CCA TgC ATT gCT TTC TTC CCA |
| | AgA Tgg |
| ! | |
| ! | A F D A V K G K C V L F P |
| 1266 | gCT TTC gAC gCT gTT AAg ggT AAg TgC gTC TTg |
| | TTC CCA |
| ! | |
| ! | Y G G C Q G N G N K F Y S |
| 1305 | TAC ggT ggT TgT CAA ggT AAC ggT AAC AAg TTC |
| | TAC TCT |
| ! | |
| ! | E K E C R E Y C G V P . . |
| 1344 | gAg AAg gAg TgT AgA gAg TAC TgT ggT gTT CCA |
| | TAg TAA |
| ! | |
| 1383 | gAATTC gC CTTAgACATg |
| ! | EcoRI |
| 1401 | ACTgTTCCTC AgTTCAAgTT gggCATTACg AgAAgACCgg |
| | TCTTgCTAgA |
| | AegI |
| 1451 | TTCTAATCAA gAggATgTCA gAATgCCATT TgCCTgAgAg |
| | ATgCAggCTT |
| 1501 | CATTTTTgAT ACTTTTTTAT TTgTAACCTA TATAgTATAg |
| | gATTTTTTTT |
| 1551 | gTCATTTTgT TTCTTCTCgT ACgAgCTTgC TCCTgATCAg |
| | CCTATCTCgC |
| 1601 | AgCTgATgAA TATCTTgTgg TAggggTTTg ggAAAATCAT |
| | TCgAgTTTgA |
| 1651 | TgTTTTTCTT ggTATTTCCC ACTCCTCTTC AgAgTACAgA |
| | AgATTAAgTg |
| 1701 | AgAAgTTCgT TTgTgCAAgC TTATCgATAA gCTTTAATgC |
| | ggTAgTTTAT |
| 1751 | CACAgTTAAA TTgCTAACgC AgTCAggCAC CgTgTATgAA |
| | ATCTAACAAT |
| 1801 | gCgCTCATCg TCATCCTCgg CACCgTCACC CTggATgCTg |
| | TAggCATAgg |
| 1851 | CTTggTTATg CCggTACTgC CgggCCTCTT gCgggATATC |
| | gTCCATTCCg |
| 1901 | ACAgCATCgC CAgTCACTAT ggCgTgCTgC TAgCgCTATA |
| | TgCgTTgATg |
| 1951 | CAATTTCTAT gCgCACCCgT TCTCggAgCA CTgTCCgACC |
| | gCTTTggCCg |
| 2001 | CCgCCCAgTC CTgCTCgCTT CgCTACTTgg AgCCACTATC |
| | gACTACgCgA |
| 2051 | TCATggCgAC CACACCCgTC CTgTggATCT ATCgAATCTA |
| | AATgTAAgTT |
| 2101 | AAAATCTCTA AATAATTAAA TAAgTCCCAg TTTCTCCATA |
| | CgAACCTTAA |
| 2151 | CAgCATTgCg gTgAgCATCT AgACCTTCAA CAgCAgCCAg |
| | ATCCATCACT |
| | XbαI |
| 2201 | gCTTggCCAA TATgTTTCAg TCCCTCAggA gTTACgTCTT |
| | gTgAAgTgAT |
| | Bsu36I |
| 2251 | gAACTTCTgg AAggTTgCAg TgTTAACTCC gCTgTATTgA |
| | CgggCATATC |
| 2301 | CgTACgTTgg CAAAgTgTgg TTggTACCgg AggAgTAATC |
| | TCCACAACTC |
| 2351 | TCTggAgAgT AggCACCAAC AAACACAgAT CCAgCgTgTT |
| | gTACTTgATC |
| 2401 | AACATAAgAA gAAgCATTCT CgATTTgCAg gATCAAgTgT |
| | TCAggAgCgT |
| 2451 | ACTgATTggA CATTTCCAAA gCCTgCTCgT AggTTgCAAC |
| | CgATAgggTT |
| 2501 | gTAgAgTgTg CAATACACTT gCgTACAATT TCAACCCTTg |
| | gCAACTgCAC |
| 2551 | AgCTTggTTg TgAACAgCAT CTTCAATTCT ggCAAgCTCC |
| | TTgTCTgTCA |
| 2601 | TATCgACAgC CAACAgAATC ACCTgggAAT CAATACCATg |
| | TTCAgCTTgA |
| 2651 | gCAgAAggTC TgAggCAACg AAATCTggAT CAgCgTATTT |
| | ATCAgCAATA |
| 2701 | ACTAgAACTT CAgAAggCCC AgCAggCATg TCAATACTAC |
| | ACAgggCTgA |
| 2751 | TgTgTCATTT TgAACCATCA TCTTggCAgC AgTAACgAAC |
| | TggTTTCCTg |
| 2801 | gACCAAATAT TTTgTCACAC TTAggAACAg TTTCTgTTCC |
| | gTAAgCCATA |
| 2851 | gCAgCTACTg CCTgggCgCC TCCTgCTAgC ACgATACACT |
| | TAgCACCAAC |
| 2901 | CTTgTgggCA ACgTAgATgA CTTCTggggT AAgggTACCA |
| | TCCTTCTTAg |
| 2951 | gTggAgATgC AAAAACAATT TCTTTgCAAC CAgCAACTTT |
| | ggCAggAACA |
| 3001 | CCCAgCATCA gggAAgTggA AggCAgAATT gCggTTCCAC |
| | CAggAATATA |
| 3051 | gAggCCAACT TTCTCAATAg gTCTTgCAAA ACgAgAgCAg |
| | ACTACACCAg |
| 3101 | ggCAAgTCTC AACTTgCAAC gTCTCCgTTA gTTgAgCTTC |
| | ATggAATTTC |
| 3151 | CTgACgTTAT CTATAgAgAg ATCAATggCT CTCTTAACgT |
| | TATCTggCAA |
| 3201 | TTgCATAAgT TCCTCTgggA AAggAgCTTC TAACACAggT |
| | gTCTTCAAAg |
| 3251 | CgACTCCATC AAACTTggCA gTTAgTTCTA AAAgggCTTT |
| | gTCACCATTT |
| 3301 | TgACgAACAT TgTCgACAAT TggTTTgACT AATTCCATAA |
| | TCTgTTCCgT |
| 3351 | TTTCTggATA ggACgACgAA gggCATCTTC AATTTCTTgT |
| | gAggAggCCT |
| | StuI |
| 3401 | TAgAAACgTC AATTTTgCAC AATTCAATAC gACCTTCAgA |
| | AgggACTTCT |
| 3451 | TTAggTTTgg ATTCTTCTTT AggTTgTTCC TTggTgTATC |
| | CTggCTTggC |
| 3501 | ATCTCCTTTC CTTCTAgTgA CCTTTAgggA CTTCATATCC |
| | AggTTTCTCT |
| 3551 | CCACCTCgTC CAACgTCACA CCgTACTTgg CACATCTAAC |
| | TAATgCAAAA |
| 3601 | TAAAATAAgT CAgCACATTC CCAggCTATA TCTTCCTTgg |
| | ATTTAgCTTC |
| 3651 | TgCAAgTTCA TCAgCTTCCT CCCTAATTTT AgCgTTCAAC |
| | AAAACTTCgT |
| 3701 | CgTCAAATAA CCgTTTggTA TAAgAACCTT CTggAgCATT |
| | gCTCTTACgA |
| 3751 | TCCCACAAgg TgCTTCCATg gCTCTAAgAC CCTTTgATTg |
| | gCCAAAACAg |
| | NcoI |
| 3801 | gAAgTgCgTT CCAAgTgACA gAAACCAACA CCTgTTTgTT |
| | CAACCACAAA |
| 3851 | TTTCAAgCAg TCTCCATCAC AATCCAATTC gATACCCAgC |
| | AACTTTTgAg |
| 3901 | TTCgTCCAgA TgTAgCACCT TTATACCACA AACCgTgACg |
| | ACgAgATTgg |
| 3951 | TAgACTCCAg TTTgTgTCCT TATAgCCTCC ggAATAgACT |
| | TTTTggACgA |
| | BspEI |
| 4001 | gTACACCAgg CCCAACgAgT AATTAgAAgA gTCAgCCACC |
| | AAAgTAgTgA |
| 4051 | ATAgACCATC ggggCggTCA gTAgTCAAAg ACgCCAACAA |
| | AATTTCACTg |
| 4101 | ACAgggAACT TTTTgACATC TTCAgAAAgT TCgTATTCAg |
| | TAgTCAATTg |
| 4151 | CCgAgCATCA ATAATggggA TTATACCAgA AgCAACAgTg |
| | gAAgTCACAT |
| 4201 | CTACCAACTT TgCggTCTCA gAAAAAgCAT AAACAgTTCT |
| | ACTACCgCCA |
| 4251 | TTAgTgAAAC TTTTCAAATC gCCCAgTggA gAAgAAAAAg |
| | gCACAgCgAT |
| 4301 | ACTAgCATTA gCgggCAAgg ATgCAACTTT ATCAACCAgg |
| | gTCCTATAgA |
| 4351 | TAACCCTAgC gCCTgggATC ATCCTTTggA CAACTCTTTC |
| | TgCCAAATCT |
| 4401 | AggTCCAAAA TCACTTCATT gATACCATTA TACggATgAC |
| | TCAACTTgCA |
| 4451 | CATTAACTTg AAgCTCAgTC gATTgAgTgA ACTTgATCAg |
| | gTTgTgCAgC |
| 4501 | TggTCAgCAg CATAgggAAA CACggCTTTT CCTACCAAAC |
| | TCAAggAATT |
| 4551 | ATCAAACTCT gCAACACTTg CgTATgCAgg TAgCAAgggA |
| | AATgTCATAC |
| 4601 | TTgAAgTCgg ACAgTgAgTg TAgTCTTgAg AAATTCTgAA |
| | gCCgTATTTT |
| 4651 | TATTATCAgT gAgTCAgTCA TCAggAgATC CTCTACgCCg |
| | gACgCATCgT |
| 4701 | ggCCggCATC ACCggCgCCA CAggTgCggT TgCTggCgCC |
| | TATATCgCCg |
| 4751 | ACATCACCgA TggggAAgAT CgggCTCgCC ACTTCgggCT |
| | CATgAgCgCT |
| 4801 | TgTTTCggCg TgggTATggT ggCAggCCCC gTggCCgggg |
| | gACTgTTggg |
| 4851 | CgCCATCTCC TTgCATgCAC CATTCCTTgC ggCggCggTg |
| | CTCAACggCC |
| 4901 | TCAACCTACT ACTgggCTgC TTCCTAATgC AggAgTCgCA |
| | TAAgggAgAg |
| 4951 | CgTCgAgTAT CTATgATTgg AAgTATgggA ATggTgATAC |
| | CCgCATTCTT |
| 5001 | CAgTgTCTTg AggTCTCCTA TCAgATTATg CCCAACTAAA |
| | gCAACCggAg |
| 5051 | gAggAgATTT CATggTAAAT TTCTCTgACT TTTggTCATC |
| | AgTAgACTCg |
| 5101 | AACTgTgAgA CTATCTCggT TATgACAgCA gAAATgTCCT |
| | TCTTggAgAC |
| 5151 | AgTAAATgAA gTCCCACCAA TAAAgAAATC CTTgTTATCA |
| | ggAACAAACT |
| 5201 | TCTTgTTTCg CgAACTTTTT CggTgCCTTg AACTATAAAA |
| | TgTAgAgTgg |
| 5251 | ATATgTCggg TAggAATggA gCgggCAAAT gCTTACCTTC |
| | TggACCTTCA |
| 5301 | AgAggTATgT AgggTTTgTA gATACTgATg CCAACTTCAg |
| | TgACAACgTT |
| 5351 | gCTATTTCgT TCAAACCATT CCgAATCCAg AgAAATCAAA |
| | gTTgTTTgTC |
| 5401 | TACTATTgAT CCAAgCCAgT gCggTCTTgA AACTgACAAT |
| | AgTgTgCTCg |
| 5451 | TgTTTTgAgg TCATCTTTgT ATgAATAAAT CTAgTCTTTg |
| | ATCTAAATAA |
| 5501 | TCTTgACgAg CCAAggCgAT AAATACCCAA ATCTAAAACT |
| | CTTTTAAAAC |
| 5551 | gTTAAAAggA CAAgTATgTC TgCCTgTATT AAACCCCAAA |
| | TCAgCTCgTA |
| 5601 | gTCTgATCCT CATCAACTTg AggggCACTA TCTTgTTTTA |
| | gAgAAATTTg |
| 5651 | CggAgATgCg ATATCgAgAA AAAggTACgC TgATTTTAAA |
| | CgTgAAATTT |
| 5701 | ATCTCAAgAT CgCggCCgCg ATCTCgAATA ATAACTgTTA |
| | TTTTTCAgTg |
| 5751 | TTCCCgATCT gCgTCTATTT CACAATACCA ACATgAgTCA |
| | gCTTATCgAT |
| 5801 | gATAAgCTgT CAAACATgAg AATTAATTCg ATgATAAgCT |
| | gTCAAACATg |
| 5851 | AgAAATCTTg AAgACgAAAg ggCCTCgTgA TACgCCTATT |
| | TTTATAggTT |
| 5901 | AATgTCATgA TAATAATggT TTCTTAgACg TACgTCAggT |
| | ggCACTTTTC |
| 5951 | ggggAAATgT gCgCggAACC CCTATTTgTT TATTTTTCTA |
| | AATACATTCA |
| 6001 | AATATgTATC CgCTCATgAg ACAATAACCC TgATAAATgC |
| | TTCAATAATA |
| 6051 | TTgAAAAAgg AAgAgTATgA gTATTCAACA TTTCCgTgTC |
| | gCCCTTATTC |
| 6101 | CCTTTTTTgC ggCATTTTgC CTTCCTgTTT TTgCTCACCC |
| | AgAAACgCTg |
| 6151 | gTgAAAgTAA AAgATgCTgA AgATCAgTTg ggTgCACgAg |
| | TgggTTACAT |
| 6201 | CgAACTggAT CTCAACAgCg gTAAgATCCT TgAgAgTTTT |
| | CgCCCCgAAg |
| 6251 | AACgTTTTCC AATgATgAgC ACTTTTAAAg TTCTgCTATg |
| | TggCgCggTA |
| 6301 | TTATCCCgTg TTgACgCCgg gCAAgAgCAA CTCggTCgCC |
| | gCATACACTA |
| 6351 | TTCTCAgAAT gACTTggTTg AgTACTCACC AgTCACAgAA |
| | AAgCATCTTA |
| 6401 | CggATggCAT gACAgTAAgA gAATTATgCA gTgCTgCCAT |
| | AACCATgAgT |
| 6451 | gATAACACTg CggCCAACTT ACTTCTgACA ACgATCggAg |
| | gACCgAAggA |
| 6501 | gCTAACCgCT TTTTTgCACA ACATgggggA TCATgTAACT |
| | CgCCTTgATC |
| 6551 | gTTgggAACC ggAgCTgAAT gAAgCCATAC CAAACgACgA |
| | gCgTgACACC |
| 6601 | ACgATgCCTg CAgCAATggC AACAACgTTg CgCAAACTAT |
| | TAACTggCgA |
| 6651 | ACTACTTACT CTAgCTTCCC ggCAACAATT AATAgACTgg |
| | ATggAggCgg |
| 6701 | ATAAAgTTgC AggACCACTT CTgCgCTCgg CCCTTCCggC |
| | TggCTggTTT |
| 6751 | ATTgCTgATA AATCTggAgC CggTgAgCgT gggTCTCgCg |
| | gTATCATTgC |
| 6801 | AgCACTgggg CCAgATggTA AgCCCTCCCg TATCgTAgTT |
| | ATCTACACgA |
| 6851 | CggggAgTCA ggCAACTATg gATgAACgAA ATAgACAgAT |
| | CgCTgAgATA |
| 6901 | ggTgCCTCAC TgATTAAgCA TTggTAACTg TCAgACCAAg |
| | TTTACTCATA |
| 6951 | TATACTTTAg ATTgATTTAA ATTgTAAACg TTAATATTTT |
| | gTTAAAATTC |
| 7001 | gCgTTAAATT TTTgTTAAAT CAgCTCATTT TTTAACCAAT |
| | AggCCgAAAT |
| 7051 | CggCAAAATC CCTTATAAAT CAAAAgAATA gACCgAgATA |
| | gggTTgAgTg |
| 7101 | TTgTTCCAgT TTggAACAAg AgTCCACTAT TAAAgAACgT |
| | ggACTCCAAC |
| 7151 | gTCAAAgggC gAAAAACCgT CTATCAgggC gATggCCCAC |
| | TACgTgAACC |
| 7201 | ATCACCCTAA TCAAgTTTTT TggggTCgAg gTgCCgTAAA |
| | gCACTAAATC |
| 7251 | ggAACCCTAA AgggAgCCCC CgATTTAgAg CTTgACgggg |
| | AAAgCCggCg |
| 7301 | AACgTggCgA gAAAggAAgg gAAgAAAgCg AAAggAgCgg |
| | gCgCTAgggC |
| 7351 | gCTggCAAgT gTAgCggTCA CgCTgCgCgT AACCACCACA |
| | CCCgCCgCgC |
| 7401 | TTAATgCgCC gCTACAgggC gCgTAAAAgg ATCTAggTgA |
| | AgATCCTTTT |
| 7451 | TgATAATCTC ATgACCAAAA TCCCTTAACg TgAgTTTTCg |
| | TTCCACTgAg |
| 7501 | CgTCAgACCC CgTAgAAAAg ATCAAAggAT CTTCTTgAgA |
| | TCCTTTTTTT |
| 7551 | CTgCgCgTAA TCTgCTgCTT gCAAACAAAA AAACCACCgC |
| | TACCAgCggT |
| 7601 | ggTTTgTTTg CCggATCAAg AgCTACCAAC TCTTTTTCCg |
| | AAggTAACTg |
| 7651 | gCTTCAgCAg AgCgCAgATA CCAAATACTg TCCTTCTAgT |
| | gTAgCCgTAg |
| 7701 | TTAggCCACC ACTTCAAgAA CTCTgTAgCA CCgCCTACAT |
| | ACCTCgCTCT |
| 7751 | gCTAATCCTg TTACCAgTgg CTgCTgCCAg TggCgATAAg |
| | TCgTgTCTTA |
| 7801 | CCgggTTggA CTCAAgACgA TAgTTACCgg ATAAggCgCA |
| | gCggTCgggC |
| 7851 | TgAACggggg gTTCgTgCAC ACAgCCCAgC TTggAgCgAA |
| | CgACCTACAC |
| 7901 | CgAACTgAgA TACCTACAgC gTgAgCATTg AgAAAgCgCC |
| | ACgCTTCCCg |
| 7951 | AAgggAgAAA ggCggACAgg TATCCggTAA gCggCAgggT |
| | CggAACAggA |
| 8001 | gAgCgCACgA gggAgCTTCC AgggggAAAC gCCTggTATC |
| | TTTATAgTCC |
| 8051 | TgTCgggTTT CgCCACCTCT gACTTgAgCg TCgATTTTTg |
| | TgATgCTCgT |
| 8101 | CAggggggCg gAgCCTATgg AAAAACgCCA gCAACgCggC |
| | CTTTTTACgg |
| 8151 | TTCCTggCCT TTTgCTggCC TTTTgCTCAC ATgTTCTTTC |
| | CTgCgTTATC |
| 8201 | CCCTgATTCT gTggATAACC gTATTACCgC CTTTgAgTgA |
| | gCTgATACCg |
| 8251 | CTCgCCgCAg CCgAACgACC gAgCgCAgCg AgTCAgTgAg |
| | CgAggAAgCg |
| 8301 | gAAgAgCgCC TgATgCggTA TTTTCTCCTT ACgCATCTgT |
| | gCggTATTTC |
| 8351 | ACACCgCATA TggTgCACTC TCAgTACAAT CTgCTCTgAT |
| | gCCgCATAgT |
| 8401 | TAAgCCAgTA TACACTCCgC TATCgCTACg TgACTgggTC |
| | ATggCTgCgC |
| 8451 | CCCgACACCC gCCAACACCC gCTgACgCgC CCTgACgggC |
| | TTgTCTgCTC |
| 8501 | CCggCATCCg CTTACAgACA AgCTgTgACC gTCTCCgggA |
| | gCTgCATgTg |
| 8551 | TCAgAggTTT TCACCgTCAT CACCgAAACg CgCgAggCAg |

TABLE 27
restriction map of pD2pick(MFαPrePro::EPI-HNE-3)
Non-cutters

| | | | | |
|---|---|---|---|---|
| AflII | ApaI | AscI | AvaI | AvrII |
| BamHI | BglII | BssHII | BstEII | MluI |
| PacI | PmlI | RsrII | SacII | SfiI |
| SnaBI | SpeI | XhoI | XmaI | |

Cutters, 3 or fewer sites

| | | | | |
|---|---|---|---|---|
| AatII | 1 | 1098 | | |
| AflIII | 1 | 8179 | | |
| AgeI | 1 | 1436 | | |
| AlwNI | 3 | 2828 | 2852 | 7765 |
| ApaLI | 3 | 6182 | 7865 | 8363 |
| AseI | 3 | 591 | 5822 | 6678 |
| BglI | 3 | 284 | 2717 | 6730 |
| BsaAI | 2 | 7191 | 8427 | |
| BsgI | 2 | 2545 | 4494 | |
| BsiWI | 3 | 1568 | 2301 | 5929 |
| BspDI | 2 | 1723 | 5795 | |
| BspEI | 1 | 3978 | | |
| BspMI | 1 | 4576 | | |
| Bst1107I | 1 | 8408 | | |
| BstBI(AsuII) | 1 | 945 | | |
| BstXI | 3 | 711 | 2765 | 2896 |
| Bsu36I | 1 | 2223 | | |
| DraIII | 2 | 3754 | 7188 | |
| EagI | 3 | 7 | 5713 | 8597 |
| Eam1105I | | 2 | 5077 | 6849 |
| Ecl136I | 1 | 216 | | |
| Eco47III | 2 | 1932 | 4795 | |
| EcoNI | 3 | 3433 | 4923 | 5295 |
| EcoRI | 1 | 1383 | | |
| EcoRV | 2 | 1885 | 5660 | |
| Esp3I(BsaI) | 2 | 3120 | 8530 | |
| EspI(Bpu1102I) | 1 | 597 | | |
| FspI | 2 | 1960 | 6629 | |
| HindIII | 3 | 885 | 1717 | 1729 |
| HpaI | 2 | 1017 | 2272 | |
| KpnI | 2 | 2323 | 2934 | |
| MscI | 2 | 2204 | 3789 | |
| NcoI | 1 | 3766 | | |
| NdeI | 1 | 8357 | | |
| NgoMI | 2 | 4702 | 7294 | |
| NheI | 2 | 1929 | 2875 | |
| NotI | 3 | 6 | 5712 | 8596 |
| NruI | 1 | 5208 | | |
| NsiI | 2 | 684 | 1241 | |
| PflMI | 2 | 196 | 1302 | |
| PmeI | 1 | 420 | | |
| PpuMI | 2 | 142 | 4339 | |
| PstI | 1 | 6608 | | |
| PvuI | 1 | 6482 | | |
| PvuII | 2 | 1600 | 4497 | |
| SacI | 1 | 216 | | |
| SalI | 1 | 3312 | | |
| ScaI | 2 | 1360 | 6371 | |
| SphI | 1 | 4863 | | |
| SspI | 3 | 2806 | 6047 | 6983 |
| StuI | 1 | 3395 | | |
| Tth111I | | 1 | 8432 | |
| XbaI | 1 | 2168 | | |
| XcmI | 1 | 711 | | |

TABLE 28
Amino-acid Sequence of ITI light chain (SEQ ID NO. 077)

```
                    111111 111122
          12345 6789012345 678901
          avlpq eeegsgggql vtevtk
2222222233333333334444444444555555555566666666667777777
2345678901234567890123456789012345678901234567890123456
KEDSCQLGYSAGPCMGMTSRYFYNGTSMACETFQYGGCMGNGNNFVTEKECL
QTC
  |--------|------------|------|----------|---|
          |-------------|------| |
                      |-----------------|
  77788
  78901
  rtvaa
                    111111111111111111111111111111111111
  888888889999999999000000000011111111112222222222333333
  234567890123456789012345678901234567890123456789012345
CNLPIVRGPCRAFIQLWAFDAVKGKCVLFPYGGCQGNGNKFYSEKECREYCGVP
  |--------|-------------|-------|-----------|--|
          |--------------|------| |
                      |------------------|
                                111111111111
                                333344444444
                                678901234567
                                gdgdeellrfsn
```

ITI-D1 comprises residues 22-76 and optionally one of residue 77, residues 77 and 78, or residues 77-79.
ITI-D2 comprises residues 80-135 and optionally one of residue 79 or residues 78-79.
The lines under the sequences represent disulfides.

TABLE 30
Physical properties of hNE inhibitors derived from
Kunitz domains

| | | | | Pre- | | | |
|---|---|---|---|---|---|---|---|
| | Par- | # | Mol | dicted | K_{D} | kₒₙ | k_{off} |
| Protein | ent | Residues | Wt | pl | (pM) | (10⁶/M/s) | (10⁻⁶/s) |
| EPI- | BPTI | 58 | 6359 | 9.10 | 2.0 | 3.7 | 7.4 |
| HNE-1 | | | | | | | |
| EPI- | BPTI | 62 | 6759 | 4.89 | 4.9 | 4.0 | 20. |
| HNE-2 | | | | | | | |
| EPI- | ITI- | 56 | 6179 | 10.04 | 6.2 | 8.0 | 50. |
| HNE-3 | D2 | | | | | | |
| EPI- | ITI- | 56 | 6237 | 9.73 | 4.6 | 10.6 | 49. |
| HNE-4 | D2 | | | | | | |

The constants K_{D} and kₒₙ above were measured with [hNE] = 8.47 × 10⁻¹⁰ molar;
k_{off} was calculated from k_{off} = K_{D} × kₒₙ.

TABLE 31
SUMMARY OF PURIFICATION OF EPI-HNE-2

| | Volume | Concentration | Total | Activity |
|---|---|---|---|---|
| STAGE | (ml) | (mg/ml) | (mg) | (mg/A₂₈₀) |
| HARVEST | 3,300 | 0.70 | 2.31 | <0.01 |
| 30K ULTRA- | 5,000 | 0.27 | 1.40 | <0.01 |
| FILTRATION | | | | |
| FILTRATE | | | | |
| 5K ULTRA- | 1,000 | 1.20 | 1.20 | 0.63 |
| FILTRATION | | | | |
| RETENTATE | | | | |
| AMMONIUM | 300 | 2.42 | 0.73 | 1.05 |
| SULFATE | | | | |
| PRECIPITATE | | | | |
| IEX pH6.2 | 98 | 6.88 | 0.67 | 1.03 |
| ELUATE | | | | |
| EPI-HNE-3, | 50 | 13.5 | 0.68 | 1.04 |
| LOT 1 | | | | |

TABLE 32
SUMMARY OF PURIFICATION OF EPI-HNE-3

| | | CONCENTRA- | | |
|---|---|---|---|---|
| | VOLUME | TION | TOTAL | ACTIVITY |
| STAGE | (ml) | (mg/ml) | (mg) | (mg/A₂₈₀) |
| HARVEST | 3,100 | 0.085 | 263 | nd |
| 30K ULTRA- | 3,260 | 0.055 | 179 | 0.007 |
| FILTRATION | | | | |
| FILTRATE | | | | |
| FIRST IEX: | 180 | 0.52 | 94 | 0.59 |
| pH6.2 | | | | |
| ELUATE | | | | |
| AMMONIUM | 100 | 0.75 | 75 | 0.59 |
| SULFATE | | | | |
| PRECIPITATE | | | | |
| IEX pH9 | 60 | 1.01 | 60 | 0.59 |
| ELUATE | | | | |
| EPI-HNE-3, | 26 | 1.54 | 40 | 0.45 |
| LOT 1 | | | | |

TABLE 33
K_{I} VALUES OF EPI-HNE PROTEINS FOR VARIOUS HUMAN
SERUM SERINE PROTEASES

| | Inhibitor: | | | |
|---|---|---|---|---|
| | EPI- | | | EPI- |
| Enzyme | HNE-1 | EPI-HNE-2 | EPI-HNE-3 | HNE-4 |
| Human Neutrophil | 2 pM | 5 pM | 6 pM | 5 pM |
| Elastase | | | | |
| Human Serum Plasmin | >6 μM | >100 μM | >100 μM | >90 μM |
| Human Serum Kallikrein | >10 μM | >100 μM | >100 μM | >90 μM |
| Human Serum Thrombin | >90 μM | >100 μM | >100 μM | >90 μM |
| Human Urine Urokinase | >90 μM | >100 μM | >100 μM | >90 μM |
| Human Plasma Factor | >90 μM | >100 μM | >100 μM | >90 μM |
| Xₐ | | | | |
| Human Pancreatic | ˜10 μM | ˜10 μM | ˜30 μM | ˜10 μM |
| Chymotrypsin | | | | |

TABLE 34
PEY-33 which produces EPI-HNE-2

| Elapse Fermenter Time | Cell Density | Activity in supernatent |
|---|---|---|
| Hours:minutes | (A₆₀₀) | (mg/l) |
| 41:09 | 89 | 28 |
| 43:08 | 89 | 57 |
| 51:54 | 95 | 92 |
| 57:05 | 120 | 140 |
| 62:43 | 140 | 245 |
| 74:45 | 160 | 360 |
| 87:56 | 170 | 473 |
| 98:13 | 190 | 656 |
| 102:25 | 200 | 678 |
| 109:58 | 230 | 710 |

Fermenter culture growth and EPI-HNE protein secretion by P. pastoris strains PEY-33. Time course is shown for fermenter cultures following initiation of methanol-limited feed growth phase. Increase in cell mass is estimated by A₆₀₀. Concentration of inhibitor protein in the fermenter culture medium was determined from measurements of hNE inhibition by diluted aliquots of cell-free CM obtained at the times indicated and stored at −20° C. until assay.

TABLE 35
PEY-43 Which produces EPI-HNE-3

| Elapse Fermenter | | |
|---|---|---|
| Time | Cell Density | Activity in supernatent |
| Hours:minutes | (A₆₀₀) | (mg/l) |
| 44:30 | 107 | 0.63 |
| 50:24 | 70 | 9.4 |
| 52:00 | 117 | 14. |
| 62:00 | 131 | 28. |
| 76:00 | 147 | 39. |
| 86:34 | 200 | 56. |
| 100:27 | 185 | 70. |
| 113:06 | 207 | 85. |

Fermenter culture growth and EPI-HNE protein secretion by P. pastoris strains PEY-43. Time course is shown for fermenter cultures following initiation of methanol-limited feed growth phase. Increase in cell mass is estimated by A₆₀₀. Concentration of inhibitor protein in the fermenter CM was determined by assays of hNE inhibition by diluted aliquots of cell-free CM obtained at the times indicated and stored at −20° C. until assay.

TABLE 36
Inhibitory properties of EPI-HNE-2

| μl of EPI-HNE-2 solution | Percent residual hNE |
|---|---|
| added | activity |
| 0. | 101.1 |
| 0. | 100.0 |
| 0. | 100.0 |
| 0. | 100.0 |
| 0. | 100.0 |
| 0. | 98.9 |
| 10. | 82.9 |
| 20. | 71.8 |
| 30. | 59.5 |
| 40. | 46.2 |
| 50. | 39.2 |
| 55. | 32.2 |
| 60. | 22.5 |
| 65. | 23.5 |
| 70. | 15.0 |
| 75. | 10.4 |
| 80. | 8.6 |
| 85. | 4.8 |
| 90. | 1.4 |
| 95. | 2.0 |
| 100. | 2.5 |
| 120. | 0.2 |
| 150. | 0.2 |
| 200. | 0.04 |

TABLE 37
hNE inhibitory properties of EPI-HNE-3

| μl of EPI-HNE-3 | Percent residual |
|---|---|
| solution added | hNE activity |
| 0. | 101.2 |
| 0. | 100.0 |
| 0. | 100.0 |
| 0. | 100.0 |
| 0. | 100.0 |
| 0. | 98.8 |
| 10. | 81.6 |
| 20. | 66.9 |
| 30. | 53.4 |
| 40. | 38.0 |
| 50. | 27.6 |
| 55. | 21.5 |
| 60. | 13.0 |
| 65. | 11.0 |
| 70. | 7.9 |
| 75. | 3.8 |
| 80. | 3.3 |
| 85. | 2.1 |
| 90. | 1.8 |
| 100. | 1.6 |
| 110. | 0.8 |
| 120. | 0.7 |
| 160. | 0.6 |
| 200. | 0.2 |

TABLE 38
pH stability of Kunitz-domain hNE inhibitors

| Incubation | Percent Residual hNE Inhibitory Activity | | | |
|---|---|---|---|---|
| pH | EPI-HNE-1 | EPI-HNE-2 | EPI-HNE-3 | EPI-HNE-4 |
| 1.0 | 102 | 98 | 97 | 98 |
| 2.0 | 100 | 97 | 97 | 100 |
| 2.6 | 101 | | | |
| 3.0 | 100 | 101 | 100 | 96 |
| 4.0 | 98 | 101 | 102 | 94 |
| 5.0 | 100 | | | |
| 5.5 | | 99 | 99 | 109 |
| 6.0 | 100 | | 103 | 99 |
| 6.5 | | | 99 | 100 |
| 7.0 | 93 | 103 | 103 | 93 |
| 7.5 | | | 87 | 109 |
| 8.0 | 96 | | 84 | 83 |
| 8.5 | | 104 | 68 | 86 |
| 9.4 | 100 | | 44 | 40 |
| 10.0 | 98 | 102 | 27 | 34 |

Proteins were incubated at 37° C. for 18 hours in buffers of defined pH (see text). In all cases protein concentrations were 1 μM. At the end of the incubation period, aliquots of the reactions were diluted and residual hNE-inhibition activity determined.

TABLE 39
Stability of hNE inhibitory proteins to oxidation by Chloramine-T

| Table 39 | | | | | | |
|---|---|---|---|---|---|---|
| Molar Ratio | Percent Residual hNE-Inhibitory Activity | | | | | |
| CHL-T: 111 | EPI- | EPI- | EPI- | EPI- | α1 anti | |
| Inhibitor | HNE-1 | HNE-2 | HNE-3 | HNE-4 | trypsin | SLPI |
| 0 | 100 | 100 | 100 | 100 | 100 | 100 |
| 0.25 | | 94 | | | | |
| 0.29 | | | | | | 93 |
| 0.30 | | | | | 97 | |
| .48 | 102 | | | | | |
| .50 | | 102 | 97 | 100 | 85 | |
| .59 | | | | | | 82 |
| .88 | | | | | | 73 |
| .95 | 100 | | | | | |
| 1.0 | | 102 | 97 | 100 | 41 | |
| 1.2 | | | | | | 65 |
| 1.4 | 98 | | | | | |
| 1.5 | | 95 | | | | |
| 1.9 | 102 | | | | | |
| 2.0 | | 102 | | | | |
| 2.1 | | | | | 7 | |
| 2.4 | | | | | | 48 |
| 3.0 | | | 97 | 100 | | |
| 3.8 | 94 | | | | | |
| 4.0 | | 95 | | | | |
| 5.0 | | | 94 | 100 | | |
| 5.2 | | | | | 7 | |
| 5.9 | | | | | | 18 |
| 9.5 | 95 | | | | | |
| 10. | | 98 | 97 | 104 | | |
| 10.4 | | | | | >5 | |
| 12. | | | | | | 15 |
| 19. | 92 | | | | | |
| 30. | | | 100 | 100 | | |
| 50. | | | 94 | 100 | | |

Inhibitors were incubated in the presence of Chloramine-T at the molar ratios indicated for 20 minutes at RT. Oxidation reactions were quenched by adding methionine to a final concentration of 4 mM. Residual hNE-inhibition activity remaining in the quenched reactions is shown as a percentage of the activity observed with no added oxidant. Proteins and concentrations in the oxidation reactions are: EPI-HNE-1, (5 μM); EPI-HNE-2, (10 μM); EPI-HNE-3, (10 μM); EPI-HNE-4, (10 μM); API, (10 μM); and SLPI, (8.5 μM).

TABLE 40
Temperature stability of EPI-HNE proteins

| Temperature | Residual hNE Inhibitory Activity | | | |
|---|---|---|---|---|
| (° C.) | EPI-HNE-1 | EPI-HNE-2 | EPI-HNE-3 | EPI-HNE-4 |
| 0 | 97 | 101 | 96 | 100 |
| 23 | 100 | 103 | 105 | 103 |
| 37 | 100 | 97 | 99 | 98 |
| 45 | 103 | | | |
| 52 | | 101 | 100 | |
| 55 | 99 | | | 98 |
| 65 | 94 | 95 | 87 | |
| 69 | | | | 82 |
| 75 | 100 | | | |
| 80 | | 101 | 79 | |
| 85 | 106 | | | 63 |
| 93 | | 88 | 57 | |
| 95 | 64 | | | 48 |

Proteins were incubated at the stated temperature for 18 hours in buffer at pH 7.0. In all cases protein concentrations were 1 μM. At the end of the incubation period, aliquots of the reactions were diluted and residual hNE-inhibition activity determined.

TABLE 41
Mutations that are likely to improve the affinity of a Kunitz domain for
hNE

Most Preferred
X18F;
[X15I(preferred), X15V];
Highly Preferred
[X16A(Preferred), X16G];
[X17F(preferred), X17M, X17L, X17I, X17L];
[{X19P, X19S}(equally preferred), X19K, X19Q];
X37G;
Preferred
X12G;
X13P;
X20R;
X21Y; X21W;
[X34V(preferred), X34P];
[X39Q, X39M];
[X32T, X32L];
[X31Q, X31E, X31V];
[X11T, X11A, X11R];
[X10Y, X10S, X10V];
[X40G, X40A];
X36G;

TABLE 42

M13_III_signal::Human_LACI-D2::mature_M13_III
DNA has SEQ ID NO. 078, amino-acid sequence has
SEQ ID NO. 079. DNA is linear and in vivo it is double
stranded.
Amino-acid sequence is of a protein that is processed in
vivo by cleavage after Ala₋₁; the entire gene encodes an
amino-acid sequence that continues to give a functional
M13 III protein.

```
    M K K L L F
  −18 −17 −16 −15 −14 −13
|atg|aaG|aaG|ctt|ctc|ttc|
          | |
          HindIII
    A I P L V V P F Y S G A
  −12 −11 −10 −9 −8 −7 −6 −5 −4 −3 −2 −1
|gcc|att|cct|ctg|gtg|gta|cct|ttc|tat|tcc|ggc|gcc|
  |BstXI | |KpnI| | KαsI|
  | XcmI |
    K P D F C F L E E D P G
    1 2 3 4 5 6 7 8 9 10 11 12
|aag|cct|gac|ttc|tgc|ttc|ctc|gag|gag|gat|ccc|ggg|
                                |XhoI | | XmαI|
    I C R G Y I T R Y F
    13 14 15 16 17 18 19 20 21 22
|att|tgc|cgc|ggt|tat|att|acg|cgt|tat|ttc|
          |SαcII| |MluI |
    Y N N Q T K Q C E R
    23 24 25 26 27 28 29 30 31 32
|tat|aat|aac|cag|act|aag|caa|tgt|gag|cgg|
                                |BsrDI| | BsrI |
    F K Y G G C L G N M
    33 34 35 36 37 38 39 40 41 42
|ttc|aag|tat|ggt|ggt|tgc|cta|ggt|aat|atg|
                                |AvrII|
    N N F E T L E E C K
    43 44 45 46 47 48 49 50 51 52
|aac|aac|ttc|gag|act|cta|gaa|gag|tgt|aag|
                          |XbαI |
    N I C E D G G A E T V E S
    53 54 55 56 57 58 100 101 102 103 104 105 106
|aac|ata|tgt|gag|gat|ggt|ggt|gct|gag|act|gtt|gag|tct|
          |NdeI | | DrdI |
```

Ala₁₀₁ is the first residue of mature M13 III.

TABLE 43

Synthetic laci-d1 with sites for cloning into display vector
DNA has SEQ ID NO. 080, amino-acid sequence has
SEQ ID NO. 081

```
        A A E M H S F C A F K A D
                    1 2 3 4 5 6 7 8 9 10
5′-gcg|gcc|gag|atg|cat|tcc|ttc|tgc|gct|ttc|aaa|gct|gat|
      |EαgI | |NsiI |
        D G P C K A I M K R
        11 12 13 14 15 16 17 18 19 20
      |gaC|ggT|ccG|tgt|aaa|gct|atc|atg|aaa|cgt|
          |RsrII| |BspHI|
        F F F N I F T R Q C
        21 22 23 24 25 26 27 28 29 30
      |ttc|ttc|ttc|aac|att|ttc|acG|cgt|cag|tgc|
                                          |MluI |
        E E F I Y G G C E G N Q
        31 32 33 34 35 36 37 38 39 40 41 42
      |gag|gaA|ttC|att|tac|ggt|ggt|tgt|gaa|ggt|aac|cag|
          |EcoRI| |BstEII|
                  N R F E S L E E
                  43 44 45 46 47 48 49 50
                |aac|cgG|ttc|gaa|tct|ctA|gag|gaa|
                  | |BstBI| |XbαI|
                  |AgeI|
        C K K M C T R D G A
        51 52 53 54 55 56 57 58 59 101
      |tgt|aag|aag|atg|tgc|act|cgt|gac|ggc gcc
                                                |KαsI |
```

Ala₁₀₁ is the first residue of mature M13 III.

TABLE 44

LACI-D1 hNE Library
DNA has SEQ ID NO. 082, amino-acid sequence has
SEQ ID NO. 083

```
        A A E M H S F C A F K A
                1 2 3 4 5 6 7 8 9
5′-gcg|gcc|gag|atg|cat|tcc|ttc|tgc|gct|ttc|aaa|gct|
      |EαgI | |NsiI |
                                                    S
            T|N T|N
        C|R K|R I|M
        S|G S|A Q|H
        Y|H E|G H|R F|L L|P
        D|N D G P|L C V|I A|G I|V F K|R R
        10 11 12 13 14 15 16 17 18 19 20
      |NRt|RVS|ggT|cNt|tgt|Rtt|gSt|Ntc|ttc|MNS|cgt|
        C
        Y|W
        F|L F F N I F T R Q C
        21 22 23 24 25 26 27 28 29 30
      |tDS|ttc|ttc|aac|att|ttc|acG|cgt|cag|tgc|
                                        |MluI |
              Q Q Q
            L|P L|P L|P
            T|K T|K T|K
        L|Q V|I V|E V|M E|G
        E|V E|A F I|A Y G G C E|A G|A N Q|R
        31 32 33 34 35 36 37 38 39 40 41 42
      |SWG|VHA|ttC|VHA|tac|ggt|ggt|tgt|VHG|gSt|aac|SRG|
              N R F E S L E E
              43 44 45 46 47 48 49 50
            |aac|cgG|ttc|gaa|tct|ctA|gag|gaa|
              | |BstBI| |XbαI|
              |AgeI|
        C K K M C T R D G A
        51 52 53 54 55 56 57 58 59 101
      |tgt|aag|aag|atg|tgc|act|cgt|gac|ggc gcc
                                                  |KαsI |
```

Variegation at 10, 11, 13, 15, 16, 17, 19, and 20 gives rise to 253,400 amino-acid sequences and 589,824 DNA sequences. Variegation at 31, 32, 34, 39, 40, and 42 gives 23,328 amino-acid and DNA sequences. There are about 5.9×10⁹ protein sequences and 1.4×10¹⁰ DNA sequences.

Ala₁₀₁ would be the first residue of mature M13 III.

TABLE 45

LACI-D2 hNE Library
DNA has SEQ ID NO. 084; amino-acid sequence has
SEQ ID NO. 085

```
                                  P|H
                                  T|N
                                C|R K|R
                                S|G S|A
                                Y|H E|G
  G A K P D F C F L E E D|N D|Q G
  −2 −1 1 2 3 4 5 6 7 8 9 10 11 12
|ggc|gcc|aag|cct|gac|ttc|tgc|ttc|ctc|gag|gag|NRt|VVS|ggg|
|KαsI| |XhoI |
                          I|N
  H|R F|L Q|M
  P|L I|V L|H C
  N|S Y|H K|P F|L
  I|T C V|I G|A N|D F T|R R Y|W F
  13 14 15 16 17 18 19 20 21 22
|MNt|tgc|Rtt|gSt|NWt|ttt|MNS|cgt|tDS|ttc|
                          Q|G
                          L|P
                          T|K
                          V|I
                      L|Q E|A
  Y N N Q A K Q C E|V R
  23 24 25 26 27 28 29 30 31 32
|tat|aat|aac|cag|Gct|aag|caa|tgt|SWg|VNA|
                        | |BsrDI|
                        |EspI |
      Q|L Q|P
      P|T T|K R|G
      V|E V|M K|E
      I|A E|A L|Q
  F K Y G G C L G|A N M|V
  33 34 35 36 37 38 39 40 41 42
|ttc|VHA|tat|ggt|ggt|tgc|VHG|gSt|aat|VBg|
  N N F E T L E E C K
  43 44 45 46 47 48 49 50 51 52
|aac|aac|ttc|gag|act|cta|gaa|gag|tgt|aag|
                          |XbαI|
  N I C E D G G A E T V E S
  53 54 55 56 57 58 100 101 102 103 104 105 106
|aac|ata|tgt|gag|gat|ggt|ggt|gct|gag|act|gtt|gag|tct|
    |NdeI| | DrdI |
```

6.37 × 10¹⁰ amino acid sequences; 1.238 × 10¹¹ DNA sequences

TABLE 46
Amino acids preferred in hNE-inhibiting Kunitz domains

| Position | Allowed amino acids |
|---|---|
| 5 | C |
| 10 | YSV, (NA) |
| 11 | TAR, (QP) |
| 12 | G |
| 13 | P, (VALI) |
| 14 | C |
| 15 | IV |
| 16 | AG |
| 17 | FM, ILV(A) |
| 18 | F |
| 19 | PS, QK |
| 20 | R |
| 21 | YW, (F) |
| 30 | C |
| 31 | QEV, (AL) |
| 32 | TL, (PSA) |
| 33 | F |
| 34 | VP |
| 35 | Y |
| 36 | G |
| 37 | G |
| 38 | C |
| 39 | MQ |
| 40 | G, A |
| 41 | N highly preferred |
| 42 | G preferred, A allowed |
| 45 | F |
| 51 | C |
| 55 | C |

TABLE 47
Cumulative collection of allowed amino acids.

| | | |
|---|---|---|
| RPDFCLEPPYTGPCvAmfpRYFYNAKAGLCQTFVYGGCmgngNNFKSAEDCMRTCCGA | EpiNE7 | (SEQ ID NO: 9) |
| RPDFCLEPPYTGPCvgffsRYFYNAKAGLCQTFVYGGCmgngNNFKSAEDCMRTCGGA | EpiNE3 | (SEQ ID NO: 10) |
| RPDFCLEPPYTGPCvgffqRYFYNAKAGLCQTFVYGGGmgnqNNFKSAEDCMRTCGGA | EpiNE6 | (SEQ ID NO: 11) |
| RPDFCLEPPYTGPCvAifpRYFYNAKAGLCQTFVYGGCmgngNNFKSAEDCMRTCCCA | EpiNE4 | (SEQ ID NO: 12) |
| RPDFCLEPPYTGPCvAffkRsFYNAKAGLCQTFVYGGCmgngNNFKSAEDCMRTCGGA | EpiNE8 | (SEQ ID NO: 13) |
| RPDFCLEPPYTGPCiAffpRYFYNAKAGLCQTFVYGGCmgngNNFKSAEDCMRTCGGA | EpiNE1 | (SEQ ID NO: 138) |
| RPDFCLEPPYTGPCiAffqRYFYNAKAGLCQTFVYGGCmgngNNFKSAEDCMRTCGCA | EpiNE5 | (SEQ ID NO: 14) |
| RPDFCLEPPYTGPCiAlfkRYFYNAKAGLCQTFVYGGCmqngNNFKSAEDCMRTCGGA | EpiNE2 | (SEQ ID NO: 15) |
| rpDfCQLGYSAGPCvaMfpRYFYNGTSMACETFQYGGCMGNGNNFVTEKDCLQTCRGA | BITI-E7 | (SEQ ID NO: 142) |
| rpDfCQLGYStGPCvaMfpRYFYNGTSMACETFQYGGCMGNGNNFVTEKDCLQTCRGA | BITI-E7-1222 | (SEQ ID NO: 143) |
| KEDfCQLGYSAGPCvaMfpRYFYNGTSMAGETFQYGGCMGNGNNFVTEKDCLQTCRGA | AMINO1 | (SEQ ID NO: 22) |
| KpDSCQLGYSAGPCvaMfpRYFYNGTSMAGETFQYGGCMGNGNNFVTEKDCLQTCRGA | AMINO2 | (SEQ ID NO: 23) |
| AACNLPIVRGPCiAFfprWAFDAVKGKCVLFPYCGCQGNGNKFYSEKECREYCGVP | EPI-hNE-3 | (SEQ ID NO: 26) |
| EACNLPIVRGPCiAFfprWAFDAVKGKCVLFPYGGCQGNGNKFYSEKECREYCGVP | EPI-hNE-4 | (SEQ ID NO: 27) |
| RPDFCLEPPYTGPCvAmfpRYFYNAKAGLCQTFlYgGCkgkGNNFKSAEDCMRTCGGA | EpiNE7.6 | (SEQ ID NO: 144) |
| RPDFCLEPPYTGPCvAmfpRYFYNAKAGLCQTFeYqGCwakGNNFKSAEDCMRTCGGA | EpiNE7.8 | (SEQ ID NO: 145) |
| RPDFCLEPPYTGPCvAmfpRYFYNAKAGLCQTFgYaGCrakGNNFKSAEDCMRTCGGA | EpiNE7.11 | (SEQ ID NO: 146) |
| RPDFCLEPPYTGPCvAmfpRYFYNAKAGLCQTFeYgGChaeGNNFKSAEDCMRTCGGA | EpiNE7.7 | (SEQ ID NO: 147) |
| RPDFCLEPPYTGPCvAmfpRYFYNAKAGLCQTFlYgGCwaqGNNFKSAEDCMRTCGGA | EpiNE7.4 | (SEQ ID NO: 148) |
| RPDFCLEPPYTGPCvAmfpRYFYNAKAGLCQTFrYgGClaeGNNFKSAEDCMRTCGGA | EpiNE7.5 | (SEQ ID NO: 149) |
| RPDFCLEPPYTGPCvAmfpRYFYNAKAGLCQTFdYgGChadGNNFKSAEDCMRTCGGA | EpiNE7.10 | (SEQ ID NO: 150) |
| RPDFCLEPPYTGPCvAmfpRYFYNAKAGLCQTFkYgGClahGNNFKSAEDCMRTCGGA | EpiNE7.1 | (SEQ ID NO: 151) |
| RPDFCLEPPYTGPCvAmfpRYFYNAKAGLCQTFtYgCCwanGNNFKSAEDCMRTCGGA | EpiNE7.16 | (SEQ ID NO: 152) |
| RPDFCLEPPYTGPCvAmfpRYFYNAKAGLCQTFnYgGCegkGNNFKSAEDCMRTCGGA | EpiNE7.19 | (SEQ ID NO: 153) |
| RPDFCLEPPYTGPCvAmfpRYFYNAKAGLCQTFqYgGCegyGNNFKSAEDCMRTCGGA | EpiNE7.12 | (SEQ ID NO: 154) |
| RPDFCLEPPYTGPCvAmfpRYFYNAKAGLCQTFhYgGCwgqGNNFKSAEDCMRTCGGA | EpiNE7.21 | (SEQ ID NO: 155) |
| RPDFCLEPPYTGPCvAmfpRYFYNAKAGLCQTFhYgGCwgeGNNFKSAEDCMRTCGGA | EpiNE7.22 | (SEQ ID NO: 156) |
| RPDFCLEPPYTGPCvAmfpRYFYNAKAGLCQTFkYgGCwgkGNNFKSAEDCMRTCGGA | EpiNE7.23 | (SEQ ID NO: 157) |
| RPDFCLEPPYTGPCvAmfpRYFYNAKAGLCQTFkYgGChgnGNNFKSAEDCMRTCGGA | EpiNE7.24 | (SEQ ID NO: 158) |
| RPDFCLEPPYTGPCvAmfpRYFYNAKAGLCQTFpYgGCwakGNNFKlAEDCMRTCGGA | EpiNE7.25 | (SEQ ID NO: 159) |
| RPDFCLEPPYTGPCvAmfpRYFYNAKAGLCGTFkYgGCwghGNNFKSAEDCMRTCGGA | EpiNE7.26 | (SEQ ID NO: 160) |
| RPDFCLEPPYTGPCvAmfpRYFYNAKAGLCQTFnYgGCwgkGNNFKSAEDCMRTCGGA | EpiNE7.27 | (SEQ ID NO: 161) |
| RPDFCLEPPYTGPCvAmfpRYFYNAKAGLCQTFtYgGClghGNNFKSAEDCMRTCGGA | EpiNE7.28 | (SEQ ID NO: 162) |
| RPDFCLEPPYTGPCvAmfpRYFYNAKAGLCQTFtYgGClgyGNNFKSAEDCMRTCGGA | EpiNE7.29 | (SEQ ID NO: 163) |
| RPDFCLEPPYTGPCvAmfpRYFYNAKAGLCQTFkYgGCwaeGNNFKSAEDCMRTCGGA | EpiNE7.30 | (SEQ ID NO: 164) |
| RPDFCLEPPYTGPCvAmfpRYFYNAKAGLCQTFgYgGCwgeGNNFKSAEDCMRTCGGA | EpiNE7.32 | (SEQ ID NO: 165) |
| RPDFCLEPPYTGPCvAmfpRYFYNAKAGLCQTFeYgGCwanGNNFKSAEDCMRTCGGA | EpiNE7.33 | (SEQ ID NO: 166) |
| RPDFCLEPPYTGPCvAmfpRYFYNAKAGLCQTFvYgGChgdGNNFKSAEDCMRTCGGA | EpiNE7.36 | (SEQ ID NO: 167) |
| RPDFCLEPPYTGPCvAmfpRYFYNAKAGLCQTFeYgGCqgkGNNFKSAEDCMRTCGGA | EpiNE7.37 | (SEQ ID NO: 168) |
| RPDFCLEPPYTGPCvAmfpRYFYNAKAGLCQTFyYgGCwakGNNFKSAEDCMRTCGGA | EpiNE7 38 | (SEQ ID NO: 169) |
| RPDFCLEPPYTGPCvAmfpRYFYNAKAGLCQTFmYgGCwgdGNNFKSAEDCMRTCCGA | EpiNE7.39 | (SEQ ID NO: 170) |
| RPDFCLEPPYTGPCvAmfpRYFYNAKAGLCQTEtYgGChqnGNNFKSAEDCMRTCGGA | EpiNE7.40 | (SEQ ID NO: 171) |
| 1111111111222222222233333333334444444444555555555 | | |
| 1234567890123456789012345678901234567890123456789012345678 | | |
| RPDFCLEPPYTGPCyAmfpRYFYNAKAGLCQTFVYGGCmgngNNFKSAEDCMRTCGGA | | (SEQ ID NO.: 173) |
| keas QLGYSA igf s wafdGTSMA EI Q qAk k VTEKe LQy Rvp | | |
| a n vr iq vk k v p k E y re | | |
| e lk L W Q | | |
| E R D | | |
| G H H | | |
| R L Y | | |
| D E | | |
| K | | |
| T | | |
| N | | |
| H | | |
| M | | |
| Y | | |
| xxxxCxxxxxxGPCxxxfxRxxxxxxxxxCxxFxYGGCxxxgNxFxxxxxCxxxCxxx | | (SEQ ID NO. 172) |

### CITATIONS

ALBR83a: Albrecht et al., Hoppe-Seyler's Z Physiol Chem (1983), 364:1697-1702.
ALBR83b: Albrecht et al., Hoppe-Seyler's Z Physiol Chem (1983), 364:1703-1708.
ALTM91: Altman et al., Protein Engineering 4(5)593-600 (1991).
AUER87: Auerswald et al., Biol Chem Hoppe-Seyler (1987), 368:1413-1425.
AUER88: Auerswald et al., Bio Chem Hoppe-Seyler (1988), 369(Supplement):27-35.
AUER89: Auerswald et al., UK Patent Application GB 2,208,511 A.
AUER90: Auerswald et al., U.S. Pat. No. 4,894,436 (16 Jan. 1990).
AUSU87: Ausubel et al., Editors. Current Protocols in Molecular Biology, Greene Publishing Associates and Wiley-Interscience, Publishers: John Wiley & Sons, New York, 1987.
BERN93: Berndt et al., J Mol Biol (1993) 234 (3) p 735-50.
BRIN90: Biol Chem Hoppe-Seyler (1990) 371 (Suppl)43-52. Brinkmann and Tschesche.
BRIN91: Eur J Biochem (1991) 202(1)95-99. Brinkmann et al.
CAMP82: J Clin Invest 70:845-852 (1982) Campbell et al.
CAMP88: J Cell Biol 106:667-676 (1988) Campbell et al.
CANT89: Cantor J O, and G M Turino, pp. 159-168 in Elastin and Elastase, Vol. II, Editors L Robert and W Hornebeck, CRC Press, Boca Raton, Fla., 1989.
DIAR90: Diarra-Mehrpour et al., Eur J Biochem (1990), 191:131-139.
DIGA89: Digan et al., (1989) Bio/Technology 7:160ff
ENGH89: Enghild et al., J Biol Chem (1989), 264:15975-15981.
GEBH86: Gebhard, W, and K Hochstrasser, pp. 389-401 in Barret and Salvesen (eds.) Protease Inhibitors (1986) Elsevier Science Publishers BV (Biomedical Division).
GEBH90: Gebhard et al., Biol Chem Hoppe-Seyler (1990), 371, suppl 13-22.
GOLD86 Am Rev Respir Dis 134:49-56 (1986) Goldstein, W, and G Doering.
GREG93: Gregg et al., Bio/Technology (1993) 11:905-910.
HEID86 Heidtmann, H, and J Travis, pp. 441-446 in Proteinase Inhibitors, Editors Barrett and Salvesen, Elsevier Science Publishers BV, Amsterdam, 1986.
HYNE90: Hynes et al., Biochemistry (1990), 29:10018-10022.
KAUM86: Kaumerer et al., Nucleic Acids Res (1986), 14:7839-7850.
MCEL91 The Lancet 337:392-4 (1991) McElvaney et al.
MCWH89 Biochem 28:5708-5714 (1989) McWherter et al.
NORR93: Norris et al., WIPO Application 93/14123.
ODOM90: Odom, L, Int J Biochem (1990), 22:925-930.
ROBE92: Roberts et al., (1992) Proc Natl Acad Sci USA 89(6)2429-33.
SALI90 TIBS 15:435-9 (November 1990) Salier, J-P.
SAMB89: Sambrook et al., Molecular Cloning, A Laboratory Manual, Second Edition, Cold Spring Harbor Laboratory, 1989.
SCHA87: Schagger, H. and G. von Jagow (1987) Analytical Biochemistry 166:368ff
SCHE67: Schecter and Berger, Biochem Biophys Res Comm (1967) 27:157-162.
SELL87: Selloum et al., Biol Chem Hoppe-Seyler (1987), 368:47-55.
SKAR92: Skarzynski, T, J Mol Biol (1992) 224(3)671-83.
SPRE94: Sprecher et al., Proc Natl Acad Sci USA 91:3353-3357 (1994).
STOL90: Stoll and Blanchard (1990) Methods in Enzymology 182:24ff.
SWAI88: Swaim, M W, and S V Pizzo, Biochem J (1988), 254:171-178.
TRAB86: Traboni, C, R Cortese, Nucleic Acids Res (1986), 14(15)6340.
TRAV88 Am J Med 84(6A)37-42 (1988) Travis.
VEDV91: Vedvick et al., J Ind Microbiol (1991) 7:197-201.
WAGN92: Wagner et al., Biochem Biophys Res Comm (1992) 186:1138-1145.

1. A non-naturally occurring protein which inhibits human neutrophil elastase and which is a protein comprising at least the core sequence of a non-naturally occurring Kunitz domain, said Kunitz domain being more similar in sequence to the core sequence 26-76 of ITI-D1 than to the core sequence 5-55 of BPTI, when its cysteines are aligned with those of BPTI and ITI-D1, but said domain differing from ITI-D1 in that at least one of the following conditions applies:
(a) the residue corresponding to BPTI residue 15 and ITI-D1 residue M36 is Val or Ile,
(b) the residue corresponding to BPTI residue 16 and ITI-D1 residue G37 is Ala,
(c) the residue corresponding to BPTI residue 18 and ITI-D1 residue T39 is Phe,
(d) the residue corresponding to BPTI residue 19 and ITI-D1 residue S40 is Pro,
(e) the residue corresponding to BPTI residue 1 and ITI-D1 residue K22, if any, is Arg,
(f) the residue corresponding to BPTI residue 2 and ITI-D1 residue E23, if any, is Pro, or
(g) the residue corresponding to BPTI residue 4 and ITI-D1 residue S25, if any, is Phe.

2. The protein of claim 1 which differs from human ITI-D1 at least one of the positions corresponding to BPTI positions 15-20.

3. The protein of claim 1 where, in said Kunitz domain, BPTI positions 1-4 are Arg-Pro-Asp-Phe (residues 1-4 of SEQ ID NO:17).

4. The protein of claim 1 where the said Kunitz domain the residue corresponding to BPTI position 31 is Glu.

5. The protein of claim 1 where the said Kunitz domain the residue corresponding to BPTI position 31 is Gln.

6. The protein of claim 1 where the said Kunitz domain the residue corresponding to BPTI position 34 is Val.

7. The protein of claim 1 where in said Kunitz domain the residue corresponding to BPTI position 4 is Phe.

8. The protein of claim 1 where in said Kunitz domain the residue corresponding to BPTI position 2 is Pro.

9. The protein of claim 1 where the said Kunitz domain the residue corresponding to BPTI position 1 is Arg.

10. The protein of claim 1 where the said Kunitz domain the residue corresponding to BPTI position 26 is Ala.

11. The protein of claim 1 where the said Kunitz domain the residue corresponding to BPTI position 18 is Phe.

12. The protein of claim 1 where in said Kunitz domain the residue corresponding to BPTI position 15 is Val or Ile, 16 is Ala or Gly, 17 is Met or Phe and 19 is Pro or Ser.

13. The protein of claim 1 which has an affinity for HNE such that its K_{D} is less than 10⁻⁸ M.

14. The protein of claim 1 which has an affinity for HNE such that its K_{D} is less than 10⁻⁹ M.

15. The protein of claim 1 which has an affinity for HNE such that its K_{D} is less than 10⁻¹¹ M.

16. The protein of claim 1 wherein both conditions (a) and (c) apply.

17. The protein of claim 16 wherein condition (d) also applies.

18. The protein of claim 1 wherein conditions (e)-(g) apply.

19. The protein of claim 16 wherein conditions (e)-(g) also apply.

20. The protein of claim 17 wherein conditions (e)-(g) also apply.

21. The protein of claim 1 where said Kunitz domain is a reference domain selected from the group consisting of BITI-E7-1222, AMINO1 (SEQ ID NO:22), AMINO2 (SEQ ID NO:23), MUTP1 (SEQ ID NO:24), BITI-E7-141 (SEQ ID NO:17), MUTT26A (SEQ ID NO:18), MUTQE (SEQ ID NO:19), and MUT1619 (SEQ ID NO:20) or a Kunitz domain comprising an amino acid sequence which otherwise differs from the core sequence of one or more of said reference domains solely by one or more class A and/or one or more class B substitutions as set forth in Table 65.

22. The protein of claim 1 where said non-naturally occurring Kunitz domain is a reference domain selected from the group consisting of BITI-E7-1222, AMINO1, AMINO2, MUTP1, BITI-E7-141, MUTT26A, MUTQE, and MUT1619 in Table 220 or a kunitz domain comprising an amino acid sequence which differs from the core sequence of one or more of said reference domains solely by one or more class A substitutions as set forth in Table 65.

23. The protein of claim 1 where the core sequence of said Kunitz domain consists of an amino acid sequence identical to that of the core sequence of a reference domain selected from the group consisting of BITI-E7-1222, AMINO1, AMINO2, MUTP1, BITI-E7-141, MUTT26A, MUTQE, and MUT1619 in Table 220.

24. The protein of claim 1 where said Kunitz domain is selected from the group consisting of BITI-E7-1222, AMINO1, AMINO2, MUTP1, BITI-E7-141, MUTT26A, MUTQE, and MUT1619 in Table 220.

25. The protein of claim 24 where said protein further comprises at least a functional portion of a coat protein of a filamentous phage, sufficient to cause display of said protein on the surface of a filamentous phage particle if said protein is expressed, together with the other proteins of said phage, in a cell capable of assembling said particles.

26. The protein of claim 25 where said coat protein is the one corresponding in said filamentous phage to the gene III protein of M13 phage.

27. The protein of claim 1 which is identical to a protein selected from the group consisting of BITI-E7-1222, AMINO1, AMINO2, MUTP1, BITI-E7-141, MUTT26A, MUTQE, and MUT1619 in Table 220.

28. The protein of claim 1 where said protein is BITI-E7-141.

29. The protein of claim 1 where said protein is MUTT26A (SEQ ID NO:18).

30. The protein of claim 1 where said protein is MUTQE (SEQ ID NO:19).

31. The protein of claim 1 where said protein is MUT1619 (SEQ ID NO:20).

32. The protein of claim 1 where said Kunitz domain is not identical in amino acid sequence to any of the Kunitz domain amino acid sequences set forth in Table 13.

33. A method of inhibiting human neutrophil elastase (HNE) which comprises contacting the HNE with an inhibitor effective amount of a protein of any one of claims 1, 12, and 14-23.

34. A method of inhibiting harmful human neutrophil elastase activity in a subject which comprises administering to the subject an inhibitorily effective amount of a protein of any one of claims 1, 12 and 14-23.

35. A method of treating emphysema in a subject which comprises administering to the subject a therapeutically effective amount of a protein of claim 1.

36. A method of treating cystic fibrosis in a subject which comprises administering to the subject a therapeutically effective amount of a protein of claim 1.
